(19) United States
(12) Patent Application Publication
(10) Pub. No.: US 2026/0021176 A1
(43) Pub. Date: Jan. 22, 2026

(54) Recombinant Non-Pathogenic Marek's Disease Virus Constructs Encoding Infectious Laryngotracheitis Virus and Infectious Bursal Disease Virus Antigens

(71) Applicant: INTERVET INC., RAHWAY, NJ (US)

(72) Inventors: Stephanie Cook, Omaha, NE (US); Mohamad Morsey, Omaha, NE (US); Ian Tarpey, St. Ives (GB); Iwan Verstegen, Boxmeer (NL); Paulus Jacobus Antonius Sondermeijer, Boxmeer (NL); Paul Vermeij, St. Anthonis (NL)

(73) Assignee: INTERVET INC., RAHWAY, NJ (US)

(21) Appl. No.: 19/295,330

(22) Filed: Aug. 8, 2025

Related U.S. Application Data
(63) Continuation of application No. 18/163,038, filed on Feb. 1, 2023.
Application No. 18/163,038 is a continuation of application No. 16/309,297, filed on Dec. 12, 2018, now Pat. No. 11,596,687.
(60) Provisional application No. 62/351,471, filed on Jun. 17, 2016.

(51) Int. Cl. A61K 39/245; A61K 39/00; A61P 31/22; C12N 7/00

(52) U.S. Cl. CPC A61K 39/245; A61P 31/22; C12N 7/00; A61K 2039/5256; A61K 2039/552; A61K 2039/70

(57) ABSTRACT

The present invention discloses novel recombinant multivalent non-pathogenic Marek's Disease virus constructs that encode and express both Infectious Laryngotracheitis Virus protein antigens and an Infectious Bursal Disease virus protein antigen, and methods of their use in poultry vaccines.

### CROSS-REFERENCE TO RELATED APPLICATIONS

This application is a continuation application of U.S. patent application Ser. No. 18/163,038, filed Feb. 1, 2023, as a continuation application of U.S. patent application Ser. No. 16/309,297 filed on Dec. 12, 2018, now U.S. Pat. No. 11,596,687, which is a national stage entry under 35 U.S.C. § 371 of PCT/EP2017/064662 filed Jun. 15, 2017, which claims priority under 35 U.S.C. § 119(e) of provisional application U.S. Ser. No. 62/351,471 filed Jun. 17, 2016, the contents of which are hereby incorporated by reference in their entireties.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, was created on Jun. 23, 2023, is named 24341-US-CNT_CL.xml and is 88,048 bytes in size.

### FIELD OF THE INVENTION

The present invention relates to novel recombinant multivalent non-pathogenic Marek's Disease virus constructs encoding and expressing Infectious Laryngotracheitis Virus and Infectious Bursal Disease Virus protein antigens, and methods of their use in poultry vaccines.

### BACKGROUND OF THE INVENTION

Pathogenic poultry viruses are not only debilitating to chickens, but they also are costly to chicken breeders because most of the resulting diseases are contagious and the poultry industry relies heavily on confined, large-scale breeding facilities. Vaccinating young chicks is often the only viable means to combat these viruses. Although attenuated or killed poultry viral vaccines remain important in the market place, in recent years significant resources have been expended on developing vaccines containing recombinant viral constructs which express pathogenic viral protein antigens. Furthermore, substantial efforts have been made to construct stable and efficacious multivalent recombinant non-pathogenic Marek's Disease virus (abbreviated as rMDVₙₚ) vectors that express foreign genes from multiple viral pathogens. Such multivalent vaccines would serve to minimize the number of injections given to the chicks and thereby, reduce discomfort and stress on the vaccinated chick, as well as significantly reduce costs in labor and materials. Vaccinating with such single multivalent constructs also would be preferable to alternative multivalent rMDVₙₚ vaccines that contain multiple recombinant monovalent rMDVₙₚ constructs, because these alternative vaccines have, at least to date, resulted in protection against only a single viral pathogen. The failure of such alternative vaccines is presumably due to one of the monovalent rMDVₙₚ constructs overgrowing the other monovalent rMDVₙₚ constructs thereby, preventing these other monovalent rMDVₙₚ constructs from inducing a significant immune response. In any case, despite substantial efforts in the past to construct stable and efficacious multivalent rMDVₙₚ vectors that express foreign genes from multiple viral pathogens indeed, such vaccines had been suggested more than twenty years ago [see e.g., U.S. Pat. No. 5,965,138], it has been only recently that a multivalent vaccine that comprises a recombinant herpesvirus of turkeys (abbreviated as rHVT) encoding antigens from more than one other pathogen has been shown to be both stable and efficacious.

One poultry virus disease that can be controlled through vaccination is Marek's disease. Marek's disease is a pathogenic disease that adversely affects chickens worldwide. Marek's disease occurs predominantly in young chickens between 2 and 5 months of age. Clinical signs include: progressive paralysis of one or more of the extremities, incoordination due to paralysis of legs, drooping of the limb due to wing involvement, and a lowered head position due to involvement of the neck muscles. In acute cases, severe depression may result. Bursal and thymic atrophy may also develop.

The etiological agent for Marek's disease is Marek's disease virus serotype 1 (abbreviated as MDV1), a cell-associated virus having a double-stranded DNA genome. MDV1 is a lymphotropic avian alphaherpesvirus that both: (i) infects B cells, which can result in cytolysis, and (ii) latently infects T cells, which can induce T-cell lymphoma. Closely related to the virulent MDV1 strain, Marek's disease virus serotype 2 (abbreviated as MDV2), previously known as Gallid herpes virus 3, is a naturally attenuated MDV strain that has been shown to have little to no pathogenicity in chickens [Petherbridge et al., J. Virological Methods 158:11-17 (2009)]. SB-1 is a specific MDV2 strain that has been shown to be useful in vaccines against MDV1 [see e.g., Murthy and Calnek, Infection and Immunity 26(2) 547-553 (1979)].

Another closely related alphaherpesvirus, Marek's disease virus serotype 3 (abbreviated as MDV3), more widely known as herpesvirus of turkeys (abbreviated as HVT), is a nonpathogenic virus of domestic turkeys [see e.g., Kingham et al., J. of General Virology 82:1123-1135 (2001)]. Two commonly used strains of HVT are the PB1 strain and the FC126 strain. Whereas, HVT is also nonpathogenic in chickens, it does induce a long-lasting protective immune response in chickens against MDV1.

Accordingly, HVT has been used in poultry vaccines against virulent MDV1 for many years, generally in combination with SB-1, which is more viraemic than HVT, but considered less safe. Alternatively, when flocks are challenged with particularly virulent MDV1 strains, HVT can be combined with the Rispen's vaccine. The Rispen's vaccine is an isolate that originated from a mildly virulent MDV1 strain that was subsequently further weakened by cell passaging. The Rispen's strain however, retains some virulence towards highly susceptible lines of chickens.

The sequence of the complete genome of HVT has been disclosed [Afonso et al., J. Virology 75(2):971-978 (2001)], and as most alphaherpesviruses, HVT possesses a significant number of potential nonessential insertion sites [see e.g., U.S. Pat. Nos. 5,187,087; 5,830,745; 5,834,305; 5,853,733; 5,928,648; 5,961,982; 6,121,043; 6,299,882 B1]. HVT also has been shown to be amenable to genetic modification and thus, has been used as a recombinant vector for many years [WO 87/04463]. Accordingly, recombinant HVT vectors have been reported to express foreign genes that encode antigens from e.g., Newcastle Disease Virus (NDV), [Sondermeijer et al., Vaccine, 11:349-358 (1993); Reddy et al., Vaccine, 14:469-477 (1996)], Infectious Bursal Disease Virus (IBDV), [Darteil et al., Virology, 211:481-490 (1995); Tsukamoto et al., J. of Virology 76(11):5637-5645 (2002)], and Infectious Laryngotracheitis Virus (ILTV) [Johnson et al., Avian Disease, 54(4):1251-1259 (2010); WO 92/03554; U.S. Pat. No. 6,875,856]. The entire genomic sequence of MDV2 is also known [see, GenBank acc. nr: AB049735.1, and Petherbridge et al., supra]. The genomic organization of the MDV2 is very similar to that of HVT, with the US region in particular, being identical to that of HVT [see, Kingham et al., supra].

In addition a recombinant chimeric virus, known as the novel avian herpesvirus (NAHV), has been constructed in which specific regions of the HVT genome have been replaced by the corresponding regions of the MDV1 genome. The NAHV also has been used to express foreign genes that encode antigens from other poultry viruses [U.S. Pat. Nos. 5,965,138; 6,913,751].

Like MDV, infectious laryngotracheitis virus (abbreviated as ILTV or ILT) is an alphaherpesvirus that adversely affects chickens, worldwide [Fuchs et al., Veterinary Research 38:261-279 (2007)]. ILTV causes acute respiratory disease in chickens, which is characterized by respiratory depression, gasping, and expectoration of bloody exudate. Viral replication is limited to cells of the respiratory tract, where in the trachea the infection gives rise to tissue erosion and hemorrhage.

Infectious bursal disease virus (abbreviated as IBDV or IBD), also called Gumboro disease virus, is the causative agent of infectious bursal disease. IBDV causes an acute, highly-contagious, viral infection of a chicken's lymphoid tissue, with its primary target being the bird's essential immunological organ: the bursa of Fabricius. The morbidity rate in susceptible flocks is high, with rapid weight loss and moderate to high mortality rates. Chicks that recover from the disease may have immune deficiencies because of destruction of (or parts of) the bursa of Fabricius. This makes them particularly vulnerable to secondary infections.

IBDV is a member of the Birnaviridae family. The viruses in this family have a genome consisting of two segments (A and B) of double-stranded RNA. Two serotypes of IBDV exist, serotype 1 and 2, which can be differentiated by virus neutralization (VN) tests. Serotype 1 viruses have been shown to be pathogenic to chickens, while serotype 2 viruses cause only sub-acute disease in turkeys. Historically, IBDV serotype 1 viruses consisted of only one type that is now known as “classic” IBD virus. More recently, so-called “variant” IBDV strains have emerged. Classic and variant strains of IBDV can be identified and distinguished by a virus neutralization test using a panel of monoclonal antibodies, or by RT-PCR [Wu et al., Avian Diseases, 51:515-526(2007)]. Well-known classic IBDV strains include, D78, Faragher 52170, and STC, whereas 89/03 is a well-known variant strain. Many live or inactivated IBDV vaccines are commercially available, e.g. a live vaccine such as NOBILIS® Gumboro D78 (MSD Animal Health).

As indicated above, because HVT can act as both an antigen that provides significant protection against Marek's Disease and as a recombinant vector, it is presently used as a platform vector for such multivalent vaccines as Innovax®-ILT (sold by Merck Animal Health), which protects against ILTV; Innovax®-ND-SB (sold by Merck Animal Health) Vectormune® HVT-NDV (sold by Ceva), both of which protect against NDV; and Vaxxitek® HVT+IBD (Merial; previously named: Gallivac™ HVT-IBD), and Vectormune™ HVT-IBD (Ceva) both of which protect against IBDV. Notably, Innovax®-ILT comprises two foreign genes, i.e., ILTV gD and ILTV gI, which has proved to be safe, effective, and stable. However, these two foreign genes are from the same pathogen and moreover, they naturally overlap and need to be co-expressed in order to allow proper immunization against ILTV. More recently, a recombinant safe, effective, and stable multivalent vaccine comprising HVT-ILTV-NDV has been disclosed [U.S. Pat. No. 8,932,604 B2 and U.S. Pat. No. 9,409,954 B2, the contents of which are hereby incorporated by reference in their entireties]. An early HVT-NDV-IBDV also has been disclosed, though upon prolonged testing during the development of the corresponding product one of the main constructs, HVP309, was found neither to display adequate genetic stability nor sustained expression of the heterologous inserts [WO 2013/057,235]. Subsequently, a more stable and efficacious construct was developed [WO 2016/102647].

Therefore, despite the clear advantages of stable, multivalent, recombinant MDVₙₚ constructs that can efficaciously express heterologous antigens from two or more different pathogens, and the substantial efforts to design them, heretofore, few have been forthcoming and even one of those few proved to be incapable of achieving all of the requisite requirements. Accordingly, the suitability of any given multivalent recombinant MDVₙₚ as a vaccine remains unpredictable when the recombinant MDVₙₚ comprises a combination of heterologous antigens that are obtained from a unique set of two or more poultry viruses. Therefore, there is a clear need to overcome the collective industry failures, by constructing novel, stable, recombinant MDVₙₚ vectors that can be used in multivalent vaccines as the sole active to protect against two or more different non-MDV1 poultry virus pathogens.

The citation of any reference herein should not be construed as an admission that such reference is available as “prior art” to the instant application.

### SUMMARY OF THE INVENTION

Accordingly, the present invention provides a novel, stable, and efficacious multivalent recombinant nonpathogenic Marek's Disease virus (rMDVₙₚ) for use as a vector to express foreign genes from multiple viral pathogens. In particular embodiments, the rMDVₙₚ is a recombinant herpesvirus of turkeys (rHVT). In alternative embodiments, the rMDVₙₚ is a recombinant Marek's disease virus serotype 2 (rMDV2). An rMDVₙₚ, e.g., an rHVT or an rMDV2, can be used in vaccines against pathogenic poultry viruses.

In particular embodiments, an rMDVₙₚ comprises a first heterologous nucleic acid located in a first nonessential site in the rMDVₙₚ genome and a second heterologous nucleic acid located in a second nonessential site in the rMDVₙₚ genome. The first heterologous nucleic acid comprises both a nucleotide sequence that encodes an Infectious Laryngotracheitis Virus (ILTV) gD protein and a nucleotide sequence that encodes an Infectious Laryngotracheitis Virus (ILTV) gI protein. The second heterologous nucleic acid comprises a nucleotide sequence that encodes an Infectious Bursal Disease Virus (IBDV) viral protein 2 (VP2). In specific embodiments of this type, the first heterologous nucleic acid comprises the nucleotide sequence of SEQ ID NO: 9 and the second heterologous nucleic acid comprises the nucleotide sequence of SEQ ID NO: 5. In specific embodiments, the rMDVₙₚ is an rHVT. In alternative embodiments, the rMDVₙₚ is an rMDV2.

In certain embodiments, the first nonessential site of the rMDVₙₚ is the US2 site, while the second nonessential site is a nonessential site of the rMDVₙₚ other than the US2 site. In related embodiments, the first nonessential site of the rMDVₙₚ is the US2 site and the second nonessential site of the rMDVₙₚ is the UL7/8 site. In yet other embodiments, the first nonessential site of the rMDVₙₚ is the US2 site and the second nonessential site of the rMDVₙₚ is the US10 site. In still other embodiments, the first nonessential site of the rMDVₙₚ is the US2 site and the second nonessential site of the rMDVₙₚ is the UL 54.5 site. In specific embodiments, the rMDVₙₚ is an rHVT. In alternative embodiments, the rMDVₙₚ is an rMDV2.

In other embodiments, the second nonessential site of the rMDVₙₚ is the US2 site, while the first nonessential site is a nonessential site of the rMDVₙₚ other than the US2 site. In related embodiments, the second nonessential site of the rMDVₙₚ is the US2 site and the first nonessential site of the rMDVₙₚ is the UL7/8 site. In yet other embodiments, the second nonessential site of the rMDVₙₚ is the US2 site and the first nonessential site of the rMDVₙₚ is the US10 site. In still other embodiments, the second nonessential site of the rMDVₙₚ is the US2 site and the first nonessential site of the rMDVₙₚ is the UL 54.5 site. In specific embodiments, the rMDVₙₚ is an rHVT. In alternative embodiments, the rMDVₙₚ is an rMDV2.

In other embodiments, the first nonessential site and the second nonessential site of the rMDVₙₚ are the same. In specific embodiments of this type, the first heterologous nucleic acid and the second heterologous nucleic acid are actually constructed as part of the same DNA molecule, which is inserted into a nonessential site of the rMDVₙₚ. Such a DNA molecule can be an expression cassette that encodes an Infectious Laryngotracheitis Virus (ILTV) gD protein, an Infectious Laryngotracheitis Virus (ILTV) gI protein, and an Infectious bursal disease virus (IBDV) VP2. In particular embodiments of this type, the DNA molecule comprises the nucleotide sequence of SEQ ID NO: 15. In other embodiments of this type, the DNA molecule comprises the nucleotide sequence of SEQ ID NO: 16. In still other embodiments of this type, the DNA molecule comprises the nucleotide sequence of SEQ ID NO: 17. In yet other embodiments of this type, the DNA molecule comprises the nucleotide sequence of SEQ ID NO: 18. In specific embodiments, the rMDVₙₚ is an rHVT. In alternative embodiments, the rMDVₙₚ is an rMDV2.

Accordingly, in particular embodiments, the first nonessential site and the second nonessential site of the rMDVₙₚ are the US2 site. In other embodiments, the first nonessential site and the second nonessential site of the rMDVₙₚ are the UL54.5 site. In yet other embodiments, the first nonessential site and the second nonessential site of the rMDVₙₚ are the UL7/8 site. In still other embodiments, the first nonessential site and the second nonessential site of the rMDVₙₚ are the US10 site. In specific embodiments, the rMDVₙₚ is an rHVT. In alternative embodiments, the rMDVₙₚ is an rMDV2.

The nucleotide sequences encoding the ILTV gD protein, the ILTV gI protein, and the IBDV VP2 protein can be operatively under the control of exogenous promoters, i.e., promoters that are not naturally found in the MDVₙₚ. In certain embodiments, these three nucleotide sequences are operatively under the control of different promoters, i.e., the nucleotide sequence encoding the ILTV gD protein is operatively under the control of a first promoter, the nucleotide sequence encoding the ILTV gI protein is operatively under the control of a second promoter, and the nucleotide sequence encoding the IBDV VP2 protein is operatively under the control of a third promoter, with the first promoter, the second promoter, and the third promoter all being different. In particular embodiments, the promoter for the nucleotide sequence encoding the ILTV gD protein is the endogenous ILTV gD promoter. In certain embodiments, the promoter for the nucleotide sequence encoding the ILTV gI protein is the endogenous ILTV gI promoter. In particular embodiments of this type, the promoter for the nucleotide sequence encoding the ILTV gD protein is the endogenous ILTV gD promoter and the promoter for the nucleotide sequence encoding the ILTV gI protein is the endogenous ILTV gI promoter. In specific embodiments, the rMDVₙₚ is an rHVT. In alternative embodiments, the rMDVₙₚ is an rMDV2.

In certain embodiments, at least one of the promoters operably linked to a nucleotide sequence encoding the ILTV gD protein, the ILTV gI protein, or the IBDV VP2 protein is the murine cytomegalovirus immediate early (mCMV IE) promoter. In related embodiments, at least one of the promoters operably linked to a nucleotide sequence encoding the ILTV gD protein, the ILTV gI protein, or the IBDV VP2 protein is the human cytomegalovirus immediate early (hCMV IE) promoter or a derivative thereof (e.g., from strain AD169). In other embodiments, at least one of the promoters operably linked to a nucleotide sequence encoding the ILTV gD protein, the ILTV gI protein, or the IBDV VP2 protein is the chicken β-actin promoter. In still other embodiments, at least one of the promoters operably linked to a nucleotide sequence encoding the ILTV gD protein, the ILTV gI protein or the IBDV VP2 protein is the pseudorabies virus (PRV) gpX promoter.

In particular embodiments, the promoter for the nucleotide sequence encoding the IBDV VP2 protein is the mCMV IE promoter. In related embodiments, the promoter for the nucleotide sequence encoding the IBDV VP2 protein is the human cytomegalovirus immediate early (hCMV IE) promoter or a derivative thereof (e.g., from strain AD169). In other embodiments, the promoter for the nucleotide sequence encoding the IBDV VP2 protein is the chicken beta-actin gene promoter. In specific embodiments, the promoter for the nucleotide sequence encoding the IBDV VP2 protein is the mCMV IE promoter, the promoter for the nucleotide sequence encoding the ILTV gD protein is the endogenous ILTV gD promoter, and the promoter for the nucleotide sequence encoding the ILTV gI protein is the endogenous ILTV gI promoter. In other specific embodiments, the promoter for the nucleotide sequence encoding the IBDV VP2 protein is the hCMV IE promoter (or a derivative thereof), the promoter for the nucleotide sequence encoding the ILTV gD protein is the endogenous ILTV gD promoter, and the promoter for the nucleotide sequence encoding the ILTV gI protein is the endogenous ILTV gI promoter. In yet other specific embodiments, the promoter for the nucleotide sequence encoding the IBDV VP2 protein is the chicken β-actin promoter, the promoter for the nucleotide sequence encoding the ILTV gD protein is the endogenous ILTV gD promoter, and the promoter for the nucleotide sequence encoding the ILTV gI protein is the endogenous ILTV gI promoter.

In certain embodiments, an rMDVₙₚ of the present invention that includes insertions of nucleotide sequences encoding the ILTV gD protein, the ILTV gI protein, and the IBDV VP2 protein also includes one or more exogenous transcription terminator sequences. In specific embodiments of this type, a transcription terminator sequence is downstream from the nucleotide sequence encoding the IBDV VP2 protein. In particular embodiments, the nucleotide sequences encoding the ILTV gD protein and the ILTV gI protein share one transcription terminator sequence and the nucleotide sequence encoding the IBDV VP2 protein has another. In particular embodiments, at least one of the transcription terminator sequences comprises a feline herpesvirus US-9 (FHV US-9) polyadenylation sequence. In related embodiments at least one of the transcription terminator sequences comprises a Herpes Simplex Virus thymidine kinase (HSV TK) polyadenylation sequence. In specific embodiments, the rMDVₙₚ is an rHVT. In alternative embodiments, the rMDVₙₚ is an rMDV2.

The present invention provides a recombinant nucleic acid comprising in 5′ to 3′ direction in the following order, (i) a murine cytomegalovirus immediate early (mCMV IE) promoter, (ii) a coding sequence for the IBDV VP2 protein, (iii) a transcription terminator sequence (iv) an ILTV gD promoter, (v) a coding sequence for the ILTV gD protein, (vi) an ILTV gI promoter, and (vii) a coding sequence for the ILTV gI protein. In a particular embodiment of this type, the recombinant nucleic acid comprises the nucleotide sequence of SEQ ID NO: 15. The present invention further provides a recombinant nucleic acid comprising in 5′ to 3′ direction in the following order, (i) a human cytomegalovirus immediate early (hCMV IE) promoter or derivative thereof, (ii) a coding sequence for the IBDV VP2 protein, (iii) a transcription terminator sequence (iv) an ILTV gD promoter, (v) a coding sequence for the ILTV gD protein, (vi) an ILTV gI promoter, and (vii) a coding sequence for the ILTV gI protein. The present invention also provides a recombinant nucleic acid comprising in 5′ to 3′ direction in the following order, (i) a chicken β-actin promoter, (ii) a coding sequence for the IBDV VP2 protein, (iii) a transcription terminator sequence (iv) an ILTV gD promoter, (v) a coding sequence for the ILTV gD protein, (vi) an ILTV gI promoter, and (vii) a coding sequence for the ILTV gI protein.

The present invention further provides a recombinant nucleic acid comprising in 5′ to 3′ direction in the following order, (i) an Infectious Laryngotracheitis Virus (ILTV) gD promoter, (ii) a coding sequence for the ILTV gD protein, (iii) an ILTV gI promoter, (iv) a coding sequence for the ILTV gI protein, (v) a human cytomegalovirus immediate early (hCMV IE), a derivative thereof (e.g., from strain AD169), or an mCMV IE promoter, (vi) a coding sequence for the IBDV VP2 protein, and (vii) a transcription terminator sequence. In a specific embodiment of this type, the recombinant nucleic acid comprises the nucleotide sequence of SEQ ID NO: 17.

The present invention further provides an rMDVₙₚ in which a recombinant nucleic acid of the present invention has been inserted into a nonessential insertion site of the rMDVₙₚ. In certain embodiments of this type, the rMDVₙₚ includes an insert in a nonessential site that comprises a recombinant nucleic acid comprising in 5′ to 3′ direction in the following order (i) a murine cytomegalovirus immediate early (mCMV IE) promoter, (ii) a coding sequence for the IBDV VP2 protein, (iii) a transcription terminator sequence (iv) an ILTV gD promoter, (v) a coding sequence for the ILTV gD protein, (vi) an ILTV gI promoter, and (vii) a coding sequence for the ILTV gI protein. In specific embodiments, intervening nucleotide sequences, such as linkers, spacer sequences, and/or extraneous coding sequences, can also be included, see Example 1 below. In a particular embodiment, the rHVT comprises the nucleotide sequence of SEQ ID NO: 15 inserted into a nonessential site. In particular embodiments of these types, the nonessential site is the US2 site. In other such embodiments, the nonessential site is the UL54.5 site. In still other such embodiments, the nonessential site is the UL7/8 site. In yet other such embodiments, the nonessential site is the US10 site. In specific embodiments, the rMDVₙₚ is an rHVT. In alternative embodiments, the rMDVₙₚ is an rMDV2.

The present invention also provides methods of making an rMDVₙₚ of the present invention. In certain embodiments, a heterologous nucleic acid is constructed that comprises a nucleotide sequence that encodes an ILTV gD protein, a nucleotide sequence that encodes an ILTV gI protein, and a nucleotide sequence that encodes an IBDV VP2 protein. The heterologous nucleic acid is then inserted into a nonessential site of an rMDVₙₚ of the present invention. In certain embodiments, the heterologous nucleic acid is an expression cassette. In particular embodiments of this type, the expression cassette comprises the nucleotide sequence of SEQ ID NO: 15. In other embodiments, a first heterologous nucleic acid is constructed that comprises a nucleotide sequence that encodes an ILTV gD protein and a nucleotide sequence that encodes an ILTV gI protein; and a second heterologous nucleic acid is constructed that comprises a nucleotide sequence that encodes an IBDV VP2 protein. The first heterologous nucleic acid is inserted into a US2 site of an rMDVₙₚ, and the second heterologous nucleic acid is inserted into an alternative nonessential site of the rMDVₙₚ. In certain embodiments, such heterologous nucleic acids are expression cassettes. In particular embodiments of this type, the first heterologous nucleic acid comprises the nucleotide sequence of SEQ ID NO: 9, and the second heterologous nucleic acid comprises the nucleotide sequence of SEQ ID NO: 5. In other embodiments of this type, the first heterologous nucleic acid comprises the nucleotide sequence of SEQ ID NO: 5, and the second heterologous nucleic acid comprises the nucleotide sequence of SEQ ID NO: 9. In specific embodiments, the method of making an rMDVₙₚ is a method of making an rHVT. In alternative embodiments, the method of making an rMDVₙₚ is a method of making an rMDV2.

The present invention further provides immunogenic compositions and/or vaccines that comprise any rMDVₙₚ of the present invention. In specific embodiments, the rMDVₙₚ is an rHVT. In alternative embodiments, the rMDVₙₚ is an rMDV2. In addition, the present invention provides methods for aiding in the protection of poultry against a disease caused by ILTV and/or IBDV and/or MDV1 by administering such a vaccine and/or immunogenic composition of the present invention. In specific embodiments, such methods aid in the protection of a chicken. In particular embodiments of this type, a vaccine of the present invention is administered subcutaneously. In other embodiments, a vaccine of the present invention is administered in ovo.

Accordingly in one aspect, the present invention provides immunogenic compositions and/or vaccines that comprise an rMDVₙₚ of the present invention. In particular embodiments these immunogenic compositions and/or vaccines are stable, safe, and have relatively strong antigen expression and/or efficacy. Alternatively, or in addition, the immunogenic compositions and/or vaccines that comprise an rMDVₙₚ of the present invention aid in the protection of a chicken against a disease caused by ILTV and/or IBDV and/or MDV1, following the administration of the immunogenic compositions and/or vaccines to the chicken.

The present invention also provides immunogenic compositions and/or vaccines that comprise any rMDVₙₚ of the present invention that is further combined with an additional IBDV, ILTV, and/or MDV antigen to improve and expand the immunogenicity provided. In addition, the present invention also provides immunogenic compositions and/or vaccines that comprise any rMDVₙₚ of the present invention that is further combined with an antigen for a pathogen other than MDV, ILTV, or NDV. In a particular embodiment of this type, the antigen is an attenuated or mild live variant IBDV (e.g., IBDV 89/03). In another particular embodiment of this type, the antigen is an attenuated (or mild live) Newcastle Disease Virus (NDV), e.g., NDV C2. The present invention also provides methods for aiding in the protection of poultry against a disease caused by ILTV and/or IBDV and/or MDV1 and/or NDV by administering such a vaccine and/or immunogenic composition to the poultry (e.g., chicken). In particular embodiments of this type, a vaccine of the present invention is administered subcutaneously. In other embodiments, a vaccine of the present invention is administered in ovo.

In certain embodiments the immunogenic compositions and/or vaccines of the present invention comprise an rHVT that comprises as an insertion into its US2 site of a recombinant nucleic acid comprising 5′ to 3′: (i) an Infectious Laryngotracheitis Virus (ILTV) gD promoter; (ii) a coding sequence for the ILTV gD protein; (iii) an ILTV gI promoter; (iv) a coding sequence for the ILTV gI protein; (v) a murine cytomegalovirus immediate early (mCMV IE) promoter; (vi) a coding sequence for the Infectious bursal disease virus VP2 protein (IBDV V2); and (vii) a transcription terminator sequence. In even more particular embodiments of this type, the recombinant nucleic acid has the nucleotide sequence of SEQ ID NO: 17. In specific embodiments of this type the immunogenic compositions and/or vaccines further comprise an attenuated (or mild live) variant infectious bursal disease virus (IBDV), e.g., IBDV is 89/03.

The present invention further provides immunogenic compositions and/or vaccines that comprise any rMDVₙₚ of the present invention combined with an additional IBDV, ILTV, and/or MDV antigen, and a pathogen other than MDV, ILTV, or IBDV.

These and other aspects of the present invention will be better appreciated by reference to the following Figures and the Detailed Description.

### BRIEF DESCRIPTION OF THE DRAWINGS

FIG. 1 is a schematic drawing of the HVT (FC126) genome, consisting of a unique long (UL) region, and a unique short (US) region, each denoted by straight lines, and flanked by repeat regions, denoted as boxes. Below the genome schematic, is a bar indicating the location of BamHI restriction enzyme digestion fragments, relative to their genome position, and the lettering nomenclature associated with each fragment. (The largest fragment was given the letter “A”, the next largest given the letter “B”, and so forth and so on). The positions of each cloned subgenomic fragment (and their designation) used to reconstruct either HVT (FC126) or the rHVT/ILT/IBDV viruses are indicated below the BamHI restriction map. The asterisk (*) indicates the position of the insertion sites: UL54.5 in 484-1050-2641-10859; US2 in 228509-ILT-435Vec6, 1333-85.B6 or 1386-04.4 #1.

FIG. 2 is a schematic drawing of four different recombinant HVTs, which depict the genes inserted into the HVT backbone and the site of their insertion. Innovaxo-ILT is an rHVT that includes an expression cassette encoding the ILTV gD and ILTV gI genes inserted in the UL54.5 site of the rHVT. 1386-48 is an rHVT that includes an expression cassette that encodes the ILTV gD, the ILTV gI, and the IBDV viral protein 2 genes inserted in the US2 site of the rHVT. 1386-134 is an rHVT that also includes both an expression cassette encoding the ILTV gD and ILTV gI, and the IBDV viral protein 2 genes inserted in the US2 site, but the cassette order is switched (i.e., VP2, then ILT gD and gI). HVT/ILT/IBDV 484 is an rHVT that includes an expression cassette that encodes the IBDV viral protein 2, the ILTV gD, and the ILTV gI genes inserted in the UL54.5 site of the rHVT.

### DETAILED DESCRIPTION OF THE INVENTION

The present invention overcomes the prior failure to be able to construct a single rMDVₙₚ vector that encodes and expresses antigens from both ILTV and IBDV. In particular embodiments, an rMDVₙₚ of the present invention encodes and expresses foreign antigens from only ILTV and IBDV, and are designed to aid in the protection against Mareks disease, Infectious Bursal Disease (Gumboro disease), and Infectious Laryngotraceitis virus. In specific embodiments, the rMDVₙₚ is an rHVT. In alternative embodiments, the rMDVₙₚ is an rMDV2. In a completely different aspect, the recombinant vector that encodes and expresses the foreign antigens from ILTV and IBDV is not an rMDVₙₚ, but rather a chimeric Marek's Disease virus that contains specified genomic sequences from MDV1 replacing their counterparts in an HVT vector, e.g., the novel avian herpes virus (NAHV) [see e.g., U.S. Pat. No. 6,913,751].

Prior to the present invention, an HVT vector already had been constructed containing an NDV gene inserted into the US10 region. This HVT-NDV vector was shown to be stable and to express sufficient levels of the corresponding NDV gene product, the NDV F protein, to protect vaccinated chickens against a virulent NDV challenge. In addition, an HVT vector already had been constructed containing a pair of ILTV genes inserted in the HVT UL54.5 region. This HVT-ILTV vector was shown to be stable and to express sufficient levels of the corresponding ILTV gene products, the ILTV gI and gD proteins, to protect vaccinated chickens against a virulent ILTV challenge virus.

Previously, a multivalent HVT construct to protect against both NDV and ILTV was designed based on the successful constructs above, i.e., inserting the NDV-F gene in the US10 site and inserting the ILTV gD and gI genes in UL54.5 site [see, U.S. Pat. No. 8,932,604 B2]. Unexpectedly however, following the passaging of this construct in tissue culture the recombinant virus lost its ability to express the ILTVgD, ILTVgI, and NDV F proteins. This proved to be true with a number of duplicate recombinant HVT constructs. Indeed, these recombinant viruses were unstable and unsuitable for further development as vaccines. These findings demonstrate that the design of a single multivalent rHVT vector that can stably express both the NDV F protein and the ILTVgD and ILTVgI proteins was not a simple process that can be extrapolated from existing information. Indeed, if such stable and efficacious multivalent rHVT vectors were possible at all, their design needed to be premised on an unpredictable set of complex interactions minimally involving the relationship between the insertion sites used and the foreign nucleotide sequences to be inserted. Accordingly, the design of rHVT constructs remains unpredictable from the known art.

The present invention therefore, provides recombinant MDVₙₚ vectors in which two genes from ILTV and one gene from IBDV have been inserted. In a particular embodiment of the present invention all three genes were inserted in the US2 region of the HVT genome. In another embodiment of the present invention all three genes were inserted in the UL54.5 site of the HVT genome. Accordingly, such rMDVₙₚ vectors should be capable to be used to provide protection against both IBDV and ILTV infections. Previously, two separate rHVT vectors were necessary to protect against these two viruses, namely one for protection against ILTV and the other for protection against IBDV.

The present invention therefore, is advantageous over current methods because it should be able to provide simultaneous protection against ILTV and IBDV by inoculation of poultry and/or poultry eggs with only a single recombinant MDVₙₚ. In particular, this allows for additional vaccines to be administered via the in ovo route, because there is a limit on how much volume can be injected into an egg, and further saves on manufacturing costs because only one rather than two vectors is needed. Moreover, this can allow an additional antigen to be included in the vaccine such as an attenuated and/or mild live NDV, e.g., strain C2.

Furthermore, the present invention includes embodiments that comprise different rMDVₙₚ constructs in the same vaccine and/or immunogenic compositions. In certain embodiments of this type, the vaccine and/or immunogenic composition comprise both an rMDV2 and an rHVT, each of which encode one or more foreign antigens. Indeed, unlike the combination of two rHVTs, which inevitably lead to one construct significantly overgrowing the other, combining an rHVT with an rMDV2 leads to no such significant overgrowth. Therefore, in specific embodiments, a vaccine of the present invention comprises an rHVT that encodes an ILTVgD protein, an ILTVgI protein, and an IBDV VP2 protein with an rMDV2 that encodes yet another poultry viral antigen, e.g., the NDV F protein.

In order to more fully appreciate the instant invention, the following definitions are provided.

The use of singular terms for convenience in description is in no way intended to be so limiting. Thus, for example, reference to a composition comprising “a polypeptide” includes reference to one or more of such polypeptides.

As used herein a “nonpathogenic Marek's Disease Virus” or “MDVₙₚ” or “npMDV” is a virus in the MDV family that shows little to no pathogenicity in poultry. The term “MDVₙₚ” includes naturally occurring MDVs that have been passaged or otherwise similarly manipulated, but does not include viral constructs in which a specific region of the genome of one MDV serotype is replaced by the corresponding region of a different MDV serotype to form a chimeric virus, such as the novel avian herpesvirus (NAHV). In certain embodiments, the MDVₙₚ is an HVT. In other embodiments, the MDVₙₚ is an MDV2. In particular embodiments of this type, the MDV2 is SB1.

As used herein, an MDVₙₚ that has been genetically modified to encode a heterologous nucleotide sequence (e.g., a foreign gene) is defined as a “recombinant MDVₙₚ” or “rMDVₙₚ”. The term “rMDVₙₚ” includes naturally occurring MDVₙₚ 's that have been genetically modified to encode a heterologous nucleotide sequence, but does not include viral constructs in which a specific region of the genome of one MDV serotype is replaced by the corresponding region of a different MDV serotype to form a chimeric virus, such as the novel avian herpesvirus (NAHV).

As used herein a “novel avian herpesvirus” (“NAHV”) is a recombinant chimeric virus comprising a unique long viral genomic region which naturally occurs in herpesvirus of turkeys virus (HVT) and a unique short viral genomic region which naturally occurs in Marek's disease 1 (MDV1) [see, U.S. Pat. Nos. 5,965,138, 6,183,753, 6,913,751 B2]. In a preferred embodiment the NAHV comprises a unique long viral genomic region which naturally occurs in herpesvirus of turkeys virus (HVT), a unique short viral genomic region which naturally occurs in Marek's disease 1 (MDV1), and the repeat viral regions of the HVT [see, U.S. Pat. No. 6,913,751 B2].

As used herein, a “nonessential site” is a site in the MDVₙₚ genome (or alternatively in the NAVH genome) in which an insertion of a heterologous nucleotide sequence into that site does not prevent the MDVₙₚ (or NAVH) from replicating in a host cell. Nonessential sites are generally identified by the gene in which they reside, e.g., the US2 site, or a region between two genes, e.g., the UL7/8 site. The use of the term “nonessential site” is in no way intended to even suggest that there is only a single unique nucleotide position in the nucleotide sequence of a given gene (or in the region between two genes) where an insertion of a heterologous nucleic acid must be made in order for the MDVₙₚ (or NAVH) to maintain its ability to replicate in a host cell.

As used herein, when an rMDVₙₚ (or NAHV) is said to comprise a given nucleic acid “inserted” in a nonessential site in the rMDVₙₚ genome (or NAHV genome), it means that the given nucleic acid is a heterologous nucleic acid that is located in that nonessential site of the MDVₙₚ (or NAHV). Accordingly, an rMDVₙₚ comprising a first nucleic acid inserted in a first nonessential site in the rMDVₙₚ genome and a second nucleic acid inserted in a second nonessential site in the rMDVₙₚ genome is equivalent to an rMDVₙₚ comprising a first heterologous nucleic acid located in a first nonessential site in the rMDVₙₚ genome and a second heterologous nucleic acid located in a second nonessential site in the rMDVₙₚ genome, and vice versa.

As used herein the term “poultry” can include chickens, turkeys, ducks, geese, quail, and pheasants.

As used herein, a “vaccine” is a composition that is suitable for application to an animal (including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a clinical disease arising from an infection with a wild-type micro-organism, i.e., strong enough for aiding in the prevention of the clinical disease, and/or preventing, ameliorating or curing the clinical disease. As established by the USDA and codified in the Title 9 Code of Federal Regulations, part 113 (9CFR 113) «Standard requirements for Animal Products» live virus vaccines must provide at least 90% protection, in the case of NDV, IBDV and ILTV, and at least 80% in the case of MDV, from clinical signs or lesions associated with the disease to obtain a license.

As used herein, a “multivalent vaccine” is a vaccine that comprises two or more different antigens. In a particular embodiment of this type, the multivalent vaccine stimulates the immune system of the recipient against two or more different pathogens.

As used herein, the term “aids in the protection” does not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, an “adjuvant” is a substance that is able to favor or amplify the cascade of immunological events, ultimately leading to a better immunological response, i.e., the integrated bodily response to an antigen. An adjuvant is in general not required for the immunological response to occur, but favors or amplifies this response.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient.

As used herein, “systemic administration” is administration into the circulatory system of the body (comprising the cardiovascular and lymphatic system), thus affecting the body as a whole rather than a specific locus such as the gastro-intestinal tract (via e.g., oral or rectal administration) and the respiratory system (via e.g., intranasal administration). Systemic administration can be performed e.g., by administering into muscle tissue (intramuscular), into the dermis (intradermal or transdermal), underneath the skin (subcutaneous), underneath the mucosa (submucosal), in the veins (intravenous) etc.

As used herein the term “parenteral administration” includes subcutaneous injections, submucosal injections, intravenous injections, intramuscular injections, intradermal injections, and infusion.

The term “approximately” is used interchangeably with the term “about” and signifies that a value is within twenty-five percent of the indicated value i.e., a peptide containing “approximately” 100 amino acid residues can contain between 75 and 125 amino acid residues.

As used herein, the term, “polypeptide” is used interchangeably with the terms “protein” and “peptide” and denotes a polymer comprising two or more amino acids connected by peptide bonds. The term “polypeptide” as used herein includes a significant fragment or segment, and encompasses a stretch of amino acid residues of at least about 8 amino acids, generally at least about 12 amino acids, typically at least about 16 amino acids, preferably at least about 20 amino acids, and, in particularly preferred embodiments, at least about 30 or more amino acids, e.g., 35, 40, 45, 50, etc. Such fragments may have ends which begin and/or end at virtually all positions, e.g., beginning at residues 1, 2, 3, etc., and ending at, e.g., 155, 154, 153, etc., in all practical combinations.

Optionally, a polypeptide may lack certain amino acid residues that are encoded by a gene or by an mRNA. For example, a gene or mRNA molecule may encode a sequence of amino acid residues on the N-terminus of a polypeptide (i.e., a signal sequence) that is cleaved from, and therefore, may not be part of the final protein.

As used herein the term “antigenic fragment” in regard to a particular protein (e.g., a protein antigen) is a fragment of that protein (including large fragments that are missing as little as a single amino acid from the full-length protein) that is antigenic, i.e., capable of specifically interacting with an antigen recognition molecule of the immune system, such as an immunoglobulin (antibody) or T cell antigen receptor. For example, an antigenic fragment of an IBDV VP2 protein is a fragment of the VP2 protein that is antigenic. Preferably, an antigenic fragment of the present invention is immunodominant for antibody and/or T cell receptor recognition.

As used herein an amino acid sequence is 100% “homologous” to a second amino acid sequence if the two amino acid sequences are identical, and/or differ only by neutral or conservative substitutions as defined below. Accordingly, an amino acid sequence is about 80% “homologous” to a second amino acid sequence if about 80% of the two amino acid sequences are identical, and/or differ only by neutral or conservative substitutions.

Functionally equivalent amino acid residues often can be substituted for residues within the sequence resulting in a conservative amino acid substitution. Such alterations define the term “a conservative substitution” as used herein. For example, one or more amino acid residues within the sequence can be substituted by another amino acid of a similar polarity, which acts as a functional equivalent, resulting in a silent alteration. Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred conservative substitutions are: Lys for Arg and vice versa such that a positive charge may be maintained; Glu for Asp and vice versa such that a negative charge may be maintained; Ser for Thr such that a free —OH can be maintained; and Gln for Asn such that a free NH₂ can be maintained. The amino acids also can be placed in the following similarity groups: (1) proline, alanine, glycine, serine, and threonine; (2) glutamine, asparagine, glutamic acid, and aspartic acid; (3) histidine, lysine, and arginine; (4) cysteine; (5) valine, leucine, isoleucine, methionine; and (6) phenylalanine, tyrosine, and tryptophan.

In a related embodiment, two highly homologous DNA sequences can be identified by their own homology, or the homology of the amino acids they encode. Such comparison of the sequences can be performed using standard software available in sequence data banks. In a particular embodiment two highly homologous DNA sequences encode amino acid sequences having about 80% identity, more preferably about 90% identity and even more preferably about 95% identity. More particularly, two highly homologous amino acid sequences have about 80% identity, even more preferably about 90% identity and even more preferably about 95% identity.

As used herein, protein and DNA sequence percent identity can be determined using software such as MacVector v9, commercially available from Accelrys (Burlington, Massachusetts) and the Clustal W algorithm with the alignment default parameters, and default parameters for identity. See, e.g., Thompson, et al, 1994. Nucleic Acids Res. 22:4673-4680. ClustalW is freely downloadable for Dos, Macintosh and Unix platforms from, e.g., EMBLI, the European Bioinformatics Institute. The present download link is found at http://www.ebi.ac.uk/clustalw/. These and other available programs can also be used to determine sequence similarity using the same or analogous default parameters.

As used herein the terms “polynucleotide”, or a “nucleic acid” or a “nucleic acid molecule” are used interchangeably and denote a molecule comprising nucleotides including, but is not limited to, RNA, cDNA, genomic DNA and even synthetic DNA sequences. The terms are also contemplated to encompass nucleic acid molecules that include any of the art-known base analogs of DNA and RNA.

A nucleic acid “coding sequence” or a “sequence encoding” a particular protein or peptide, is a nucleotide sequence which is transcribed and translated into a polypeptide in vitro or in vivo when placed under the control of appropriate regulatory elements.

The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., avian) DNA, and even synthetic DNA sequences. A transcription termination sequence can be located 3′ to the coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

As used herein, the term “transcription terminator sequence” is used interchangeably with the term “polyadenylation regulatory element” and is a sequence that is generally downstream from a DNA coding region and that may be required for the complete termination of the transcription of that DNA coding sequence. A transcription terminator is a regulatory DNA element involved in the termination of the transcription of a coding region into RNA. Generally, such an element encodes a section, e.g. a hairpin structure, which has a secondary structure that can cause the RNA polymerase complex to stop transcription. A transcription terminator is therefore always situated downstream of the stop codon from the region to be translated, the 3′ untranslated region.

As used herein an “expression cassette” is a recombinant nucleic acid that minimally comprises a promoter and a heterologous coding sequence operably linked to that promoter. In many such embodiments, the expression cassette further comprises a transcription terminator sequence. Accordingly, the insertion of an expression cassette into a nonessential site of the rMDVₙₚ genome can lead to the expression of the heterologous coding sequence by the rMDVₙₚ. In specific embodiments, the rMDVₙₚ is an rHVT. In alternative embodiments, the rMDVₙₚ is an rMDV2.

A “heterologous nucleotide sequence” as used herein is a nucleotide sequence that is added to a nucleotide sequence of the present invention by recombinant methods to form a nucleic acid that is not naturally formed in nature. In specific embodiments, a “heterologous nucleotide sequence” of the present invention can encode a protein antigen such as the IBDV VP2 protein, the ILTV gI protein, and/or the ILTV gD protein. Heterologous nucleotide sequences can also encode fusion (e.g., chimeric) proteins. In addition, a heterologous nucleotide sequence can encode peptides and/or proteins that contain regulatory and/or structural properties. In other such embodiments, a heterologous nucleotide sequence can encode a protein or peptide that functions as a means of detecting the protein or peptide encoded by the nucleotide sequence of the present invention after the recombinant nucleic acid is expressed. In still another embodiment, the heterologous nucleotide sequence can function as a means of detecting a nucleotide sequence of the present invention. A heterologous nucleotide sequence can comprise non-coding sequences including restriction sites, regulatory sites, promoters and the like. A “heterologous nucleic acid” comprises a heterologous nucleotide sequence.

Insertion of a nucleic acid encoding an antigen of the present invention into an rMDVₙₚ vector is easily accomplished when the termini of both the nucleic acid and the vector comprise compatible restriction sites. If this cannot be done, it may be necessary to modify the termini of the nucleotide sequence and/or vector by digesting back single-stranded nucleic acid overhangs (e.g., DNA overhangs) generated by restriction endonuclease cleavage to produce blunt ends, or to achieve the same result by filling in the single-stranded termini with an appropriate polymerase. Alternatively, desired sites may be produced, e.g., by ligating nucleotide sequences (linkers) onto the termini. Such linkers may comprise specific oligonucleotide sequences that define desired restriction sites. Restriction sites can also be generated through the use of the polymerase chain reaction (PCR). [See, e.g., Saiki et al., Science 239:487-491 (1988)]. The cleaved vector and the DNA fragments may also be modified, if required, by homopolymeric tailing.

### Protein Antigens and Nucleic Acids Encoding the Protein Antigens

The ILTV gD gene appears to encode a glycoprotein of 434 amino acids in length having a molecular weight of 48,477 daltons, although others have suggested that a downstream start codon, which leads to an ILTV gD protein comprising only 377 amino acid residues, is the actual start codon [Wild et al., Virus Genes 12:104-116 (1996)]. The ILTV gI gene encodes a glycoprotein of 362 amino acids in length having a molecular weight of 39,753 daltons [U.S. Pat. No. 6,875,856, hereby incorporated by reference]. Nucleic acids encoding natural and/or laboratory derived variants of the ILTV gD and ILTV gI may be substituted for those presently exemplified.

In particular embodiments of the present invention, an rMDVₙₚ comprises a recombinant nucleic acid (e.g., an expression cassette) that encodes an ILTV gD protein comprising the amino acid sequence of SEQ ID NO: 2 or an antigenic fragment thereof. In related embodiments the rMDVₙₚ comprises a recombinant nucleic acid that encodes an ILTV gD protein comprising an amino acid sequence that has greater than 90%, and/or greater than 95%, and/or greater than 98%, and/or greater than 99% identity to the amino acid sequence of SEQ ID NO: 2. In particular embodiments, the ILTV gD protein is encoded by the nucleotide sequence of SEQ ID NO: 1. In specific embodiments, the rMDVₙₚ is an rHVT. In alternative embodiments, the rMDVₙₚ is an rMDV2.

In certain embodiments of the present invention, an rMDVₙₚ comprises a recombinant nucleic acid (e.g., an expression cassette) that encodes an ILTV gI protein comprising the amino acid sequence of SEQ ID NO: 4 or an antigenic fragment thereof. In related embodiments, the rMDVₙₚ comprises a recombinant nucleic acid that encodes an ILTV gI protein comprising an amino acid sequence that has greater than 90%, and/or greater than 95%, and/or greater than 98%, and/or greater than 99% identity to the amino acid sequence of SEQ ID NO: 4. In particular embodiments, the ILTV gI protein is encoded by the nucleotide sequence of SEQ ID NO: 3. In specific embodiments, the rMDVₙₚ is an rHVT. In alternative embodiments, the rMDVₙₚ is an rMDV2.

As mentioned earlier, IBDV is a member of the Birnaviridae family, which has a genome consisting of two segments (A and B) of double-stranded RNA. The larger segment A encodes a polyprotein of 110 kDa, which is subsequently cleaved by autoproteolysis to form mature viral proteins VP2, VP3 and VP4. Of these, VP2 and VP3 are the structural capsid proteins for the virion, with VP2 protein being the major host-protective immunogen. In the case of IBDV, two serotypes exist, serotype 1 and 2 which can be distinguished by virus neutralization (VN) tests. Serotype 1 viruses have been shown to be pathogenic to chickens, while serotype 2 IBDV only causes sub-acute disease in turkeys. Historically, IBDV serotype 1 viruses consisted of only one type that is known as “classic” IBD virus, but subsequently, so-called “variant” IBDV strains have emerged. In particular embodiments of the present invention the IBDV VP2 gene encodes a VP2 protein from an IBDV that is of the classic type. Such genes are well known and their sequence information is readily available, [see e.g., GenBank acc.nr: D00869 (F52/70), D00499 (STC), or AF499929 (D78)]. Alternatively, this gene can be obtained from the genome of a classic IBDV isolated from nature, using routine techniques for manipulating a Birnavirus. Classic type IBDV's can be readily identified using serology, or molecular biology.

In particular embodiments of the present invention, an rMDVₙₚ comprises a recombinant nucleic acid (e.g., an expression cassette) that encodes an IBDV VP2 protein comprising the amino acid sequence of SEQ ID NO: 6 or an antigenic fragment thereof. In related embodiments, the rMDVₙₚ comprises a recombinant nucleic acid that encodes an IBDV VP2 protein comprising an amino acid sequence that has greater than 90%, and/or greater than 95%, and/or greater than 98%, and/or greater than 99% identity to the amino acid sequence of SEQ ID NO: 6. In specific embodiments, the IBDV VP2 protein is encoded by the nucleotide sequence of SEQ ID NO: 5. In specific embodiments, the rMDVₙₚ is an rHVT. In alternative embodiments, the rMDVₙₚ is an rMDV2.

Routine vaccinations against IBDV are performed as early as possible in the life of poultry using attenuated IBDV strains, but these can only be applied when the level of MDA against IBDV has decreased enough, which commonly is somewhere between 15 and 20 days post hatch. Many ‘live’ or inactivated IBDV vaccines are commercially available, e.g., a ‘live’ vaccine such as Nobilis™ Gumboro D78 (Merck Animal Health).

NDV has a non-segmented, negative sense, single stranded RNA genome, which is about 15 kb in size, and contains six genes, amongst which is the NDV F protein gene which encodes the so-called “fusion” glycoprotein (F protein). The F protein is involved in NDV's attachment of and entry into host cells, and as the immunodominant protein it can be the basis of an effective immune response against NDV. The NDV F protein is expressed as a native F0 protein, which is activated upon cleavage by extra-cellular peptidases.

An NDV F protein gene, for example, can be derived from NDV Clone 30, a common lentogenic NDV vaccine strain. Nucleic acids encoding natural and/or laboratory derived variants of the F protein gene would equally be applicable, either from lentogenic, mesogenic or velogenic NDV, as the F protein gene sequence itself is highly conserved in these different NDV pathotypes.

### Promoters and Polyadenylation Regulatory Elements

A promoter is a functional region on the genome of an organism that directs the transcription of a downstream coding region. A promoter is therefore situated upstream of the coding region of a gene. The mRNA synthesis directed by the promoter starts from the ‘transcription start site’ (TSS). The mRNA produced is in turn translated into protein starting from the gene's start codon, which is the first ATG sequence in the open reading frame (the first AUG in the mRNA). Typically the TSS is located at 30-40 nucleotides upstream of the start codon. A TSS can be determined by sequencing the 5′ end of the mRNA of a gene, e.g. by the RACE technique. In general promoters are comprised within about 1000 nucleotides upstream of the position of the A of the start codon, which is generally denoted as A+1, and most promoters are situated between nucleotides −500 and A+1.

The nomenclature for a promoter is commonly based on the name of gene that it controls the expression of. For example, the murine cytomegalovirus immediate early 1 gene (mCMV-IE1) promoter “mCMV-IE1 gene promoter”, refers to the promoter that naturally drives the expression of the early 1 gene (IE1 gene) for mCMV and accordingly, is situated immediately upstream of that gene. Because the IE1-gene is such a well-documented and clearly recognizable gene, and because the genomes of several mCMVs have been sequenced (in whole or in part), such a promoter readily can be identified by routine techniques. For example, in a basic protocol a promoter can be obtained by roughly sub-cloning the region in between two consecutive genes, e.g. from the poly A signal of an upstream gene to the TSS of a downstream gene. The promoter then can be identified by standard tests, e.g., by the expression of a marker gene by progressively smaller sections of a suspected promoter.

Generally, promoters contain a number of recognizable regulatory regions, such as an enhancer region, which is involved in binding regulatory factors that influence the time, the duration, the conditions, and the level of transcription. Whereas the enhancer region is normally situated upstream, a promoter also contains a region more downstream that is involved in the binding of transcription factors and directing RNA polymerase itself. This downstream region generally contains a number of conserved promoter sequence elements such as the TATA box, the CAAT box, and the GC box.

A promoter comprising both the enhancer- and the downstream region is termed a “complete” promoter, whereas a promoter comprising only the downstream region, is termed a “core” promoter.

A promoter for the expression of a (heterologous) gene in a (virus) vector needs to be able to effectively drive the transcription of that downstream coding sequence. This is generally referred to as the promoter being “operatively linked” to the coding sequence, such that the gene is ‘under the control’ of the promoter, or is ‘driven by’ the promoter. This generally means that in an expression cassette the promoter and the coding sequence of the gene are found on the same nucleic acid, in effective proximity, and with no signals or sequences between them that would intervene with effective transcription of the coding sequence.

The mCMV-IE1 gene promoter is well known in the art, and can be readily obtained from a variety of commercial sources, such as from suppliers of commercial plasmids for cloning and expression. The IE1 gene is also called the ‘major IE gene’. The mCMV-IE1 protein has also been referred to as pp89. Dörsch-Häsler et al. [Proc. Nat. Acad. Sci., 82:8325-8329 (1985)] described the mCMV IE1 gene promoter in 1985, and the use of this promoter in heterologous expression is also described in WO 87/03.905 and EP 728,842. The nucleotide sequence of the complete mCMV IE locus is available from GenBank under acc. nr. L06816.1 (from March 2004). The mCMV itself is available from the ATCC: initially under acc. nr. VR-194, and more recently this has been continued under acc. nr. VR-1399.

In one embodiment of the invention, the mCMV-IE1 gene promoter is a complete promoter, comprising both the core promoter region, as well as the enhancer region for the mCMV-IE1 gene. The complete mCMV-IE1 gene promoter is about 1.4 kb in size. However, the present invention also allows for some variance in length of not only the mCMV IE1-gene promoter, but also of the other elements that make up the recombinant DNA expression cassette employed in the present invention. This can result from differences in the exact conditions that are used for cloning and construction. For example, this variance may arise from using different restriction enzyme sites, PCR cloning primers, or different conditions for adapting the ends of the cloning molecules used. Consequently, some variation in length—smaller or larger—of the constituting elements may occur, without affecting the stability, and relatively strong antigen expression and/or efficacy of the overall expression cassette. In that case these length differences are immaterial, and are within the scope of the invention. Therefore, an mCMV-IE1 gene promoter of “about 1.4 kb” is: 1.4 kb±about 25%. In particular embodiments the promoter is 1.4 kb±about 20%. In still other embodiments the variance can be 1.4 kb±about 15%, 1.4 kb±about 12%, 1.4 kb±about 10%, 1.4 kb±about 8%, 1.4 kb±about 6%, 1.4 kb±about 5%, 1.4 kb±about 4%, 1.4 kb±about 3%, 1.4 kb±about 2%, or even 1.4 kb±about 1%.

Similarly, homologs or variants of the promoter element may be used that are equally effective and stable. Therefore, in certain embodiments the mCMV-IE1 gene promoter of the present invention can be a DNA molecule of about 1.4 kb that comprises a nucleotide sequence with at least 95%, 96%, 97%, 98%, or even 99% nucleotide sequence identity to the nucleotide sequence of SEQ ID NO: 10. In a particular embodiment the mCMV-IE1 gene promoter consists of nucleotide sequence of SEQ ID NO: 10.

Many alternative promoters can be used to drive the expression of a heterologous gene encoding a protein antigen or antigenic fragment thereof in an rMDVₙₚ of the present invention. Examples include the pseudorabies virus (PRV) gpX promoter [see, WO 87/04463], the Rous sarcoma virus LTR promoter, the SV40 early gene promoter, the chicken beta-actin gene promoter comprising the nucleotide sequence of SEQ ID NO: 11, the Towne Strain hCMV IE promoter, a derivative of the hCMV IE promoter (from strain AD169) comprising the nucleotide sequence of SEQ ID NO: 12, an ILTV gD promoter comprising the nucleotide sequence of SEQ ID NO: 7, and an ILTV gI promoter comprising the nucleotide sequence of SEQ ID NO: 8, [see e.g., U.S. Pat. No. 6,183,753 B1], the human cytomegalovirus immediate early1 (hCMV IE1) gene promoter [U.S. Pat. Nos. 5,830,745; 5,980,906], and the chicken beta-actin gene promoter [EP 1 298 139 B1]. A particular heterologous promoter for the IBDV VP2 gene is the murine (mCMV IE1) cytomegalovirus promoter. In a particular embodiment of this type the mCMV IE1 comprises the nucleotide sequence of SEQ ID NO: 10 [see e.g., EP 728,842; PCT/EP2015/081121].

The inclusion of a polyadenylation regulatory element downstream from a DNA coding region is oftentimes required to terminate the transcription of the coding DNA sequence. Accordingly, many genes comprise a polyadenylation regulatory element at the downstream end of their coding sequence. Many such regulatory elements have been identified and can be used in an rMDVₙₚ of the present invention. Specific examples of polyadenylation regulatory elements as exemplified herein, include a FHV US-9 polyadenylation signal comprising the nucleotide sequence of SEQ ID NO: 13, and the HSV thymidine kinase polyadenylation signal comprising the nucleotide sequence of SEQ ID NO: 14.

### Vaccines and Immunogenic Compositions

The present invention relates to the use of the recombinant MDVₙₚ, the nucleic acid molecules used to construct the MDVₙₚ, or the host cells to grow them, or any combination thereof, all according to the present invention for the manufacture of a vaccine for poultry. Accordingly, the present invention provides vaccines and/or immunogenic compositions that include a multivalent recombinant MDVₙₚ of the present invention. Such vaccines can be used to aid in the prevention and/or prevent Infectious Bursal Disease (Gumboro disease), and/or Marek's disease, and/or maladies associated with ILTV infections. A vaccine according to the present invention can be used for prophylactic and/or for therapeutic treatment, and thus can interfere with the establishment and/or with the progression of an infection and/or its clinical symptoms of disease.

A recombinant MDVₙₚ of the present invention can be grown by any number of means currently practiced in the field. For example, a recombinant MDVₙₚ of the present invention can be grown through the use of in vitro cultures of primary chicken cells, see e.g., the Examples below where chicken embryo fibroblast cells (CEFs) were used. The CEFs can be prepared by trypsinization of chicken embryos. The CEFs also can be plated in monolayers and then infected with the MDVₙₚ. This particular process can be readily scaled up to industrial-sized production.

Therefore, a further aspect of the invention relates to a method for the preparation of the vaccine according to the invention comprising the steps of infecting host cells with a recombinant MDVₙₚ of the present invention, harvesting the infected host cells, and then admixing the harvested infected host cells with a pharmaceutically acceptable carrier. Suitable methods for infection, culture and harvesting are well known in the art and are described and exemplified herein.

Typically, the infected host cells are harvested while still intact to obtain the recombinant MDVₙₚ in its cell-associated form. These cells can be taken up in an appropriate carrier composition to provide stabilization for storage and freezing. The infected cells can be filled into glass ampoules, which are sealed, frozen and stored in liquid nitrogen. Accordingly, in certain embodiments of the present invention, the vaccines and/or immunogenic compositions of the present invention are stored frozen and accordingly, comprise a cryropreservative, such as dimethyl sulfoxide (DMSO), to preserve the frozen infected cells.

Alternatively, when the recombinant MDVₙₚ is a recombinant HVT, it can be isolated from its host cell, for instance through sonication at the end of culturing, and then taken up into a stabilizer, and freeze-dried (lyophilized) for stable storage or otherwise reduced in liquid volume, for storage, and then reconstituted in a liquid diluent before or at the time of administration. Such reconstitution may be achieved using, for example, vaccine-grade water. In certain embodiments, a lyophilized portion of a multivalent vaccine can comprise one or more antigens and the diluent can comprise one or more other antigens.

In particular embodiments a vaccine of the present invention (or a portion thereof) can be in a freeze-dried form, e.g., as tablets and/or spheres that are produced by a method described in WO 2010/125084, hereby incorporated by reference in its entirety. In particular, reference is made to the examples, from page 15, line 28 to page 27, line 9 of WO 2010/125084, describing a method to produce such fast disintegrating tablets/spheres. Such freeze-dried forms can be readily dissolved in a diluent, to enable systemic administration of the vaccine.

Vaccines and immunogenic compositions can, but do not necessarily include, physiologically compatible buffers and saline and the like, as well as pharmaceutically acceptable adjuvants. Adjuvants can be useful for improving the immune response and/or increasing the stability of vaccine preparations. Adjuvants are typically described as non-specific stimulators of the immune system, but also can be useful for targeting specific arms of the immune system. One or more compounds which have this activity may be added to the vaccine. Therefore, particular vaccines of the present invention can further comprise an adjuvant. Examples of chemical compounds that can be used as adjuvants include, but are not limited to aluminum compounds (e.g., aluminum hydroxide), metabolizable and non-metabolizable oils, mineral oils including mannide oleate derivatives in mineral oil solution (e.g., MONTANIDE ISA 70 from Seppic SA, France), and light mineral oils such as DRAKEOL 6VR, block polymers, ISCOM's (immune stimulating complexes), vitamins and minerals (including but not limited to: vitamin E, vitamin A, selenium, and vitamin B12) and CARBOPOL®.

Other suitable adjuvants, which sometimes have been referred to as immune stimulants, include, but are not limited to: cytokines, growth factors, chemokines, supernatants from cell cultures of lymphocytes, monocytes, cells from lymphoid organs, cell preparations and/or extracts from plants, bacteria or parasites (Staphylococcus aureus or lipopolysaccharide preparations) or mitogens. Generally, an adjuvant is administered at the same time as an antigen of the present invention. However, adjuvants can also or alternatively be administered within a two-week period prior to the vaccination, and/or for a period of time after vaccination, i.e., so long as the antigen, e.g., a recombinant MDVₙₚ of the present invention persists in the tissues.

The vaccines and/or immunogenic compositions of the present invention may be administered by any route such as in ovo, by parenteral administration, including intramuscular injection, subcutaneous injection, intravenous injection, intradermal injection, by scarification, by oral administration, or by any combination thereof.

Furthermore, the multivalent recombinant MDVₙₚ of the present invention can be used and/or combined with additional IBDV, ILTV, and/or MDV antigens to improve and expand the immunogenicity provided, and/or antigens for other pathogens (e.g., NDV) in order to provide immune protection against such other pathogens. These additional antigens can be either live or killed whole microorganisms, other recombinant vectors, cell homogenates, extracts, proteins, or any other such derivative, provided that they do not negatively interfere with the safety, and stability with relatively strong antigen expression and/or efficacy of the vaccine according to the present invention.

The combination of a multivalent recombinant MDVₙₚ of the present invention with an additional MDV, IBDV, and/or ILTV antigen can be advantageous in those cases in which very virulent field strains of MDV, IBDV, or ILTV are prevalent, e.g., in a particular geographic region. In this regard, the combination of a multivalent recombinant MDVₙₚ of the present invention with an MDV1, MDV2, or HVT includes the Rispens (MDV1) strain, the SB1 (MDV2) strain, the FC-126 (HVT) strain and/or PB1 (HVT) strain. To improve the response against IBDV, multivalent recombinant MDVₙₚ may be combined with an IBDV vaccine strain, such as a mild live IBDV vaccine strain, e.g., D78 (cloned intermediate strain), PBG98, Cu-1, ST-12 (an intermediate strain), or 89/03 (a live Delaware variant strain) in a multivalent vaccine.

Examples of other microorganisms that can be used as antigens together with the multivalent recombinant MDVₙₚ of the present invention include: (i) viruses such as
infectious bronchitis virus, adenovirus, egg drop syndrome virus, infectious bursal disease virus, chicken anaemia virus, avian encephalo-myelitis virus, fowl pox virus, turkey rhinotracheitis virus, duck plague virus (duck viral enteritis), pigeon pox virus, avian leucosis virus, avian pneumovirus, and reovirus, (ii) bacteria, such as Escherichia coli, Salmonella spec., Ornitobacterium rhinotracheale, Haemophilis paragallinarum, Pasteurella multocida, Erysipelothrix rhusiopathiae, Erysipelas spec., Mycoplasma spec., and Clostridium spec., (iii) parasites such as Eimeria spec., and (iv) fungi, such as Aspergillus spec. In particular embodiments of the present invention, a recombinant MDVₙₚ of the present invention can be combined with a mild live NDV vaccine strain such as vaccine strain C2. Many of such strains are used in commercial vaccines.

The combination vaccine can be made in a variety of ways including by combining the recombinant MDVₙₚ of the present invention with preparations of virus, or bacteria, or fungi, or parasites, or host cells, or a mixture of any and/or all of these. In particular embodiments, the components for such a combination vaccine are conveniently produced separately and then combined and filled into the same vaccine container.

As described above, a vaccine according to the invention can be used advantageously to provide safe and effective immune protection in poultry to a multiple diseases, by a single inoculation at very young age or in ovo. Alternatively, as would be apparent to anyone skilled in the art of poultry vaccines the combinations described above also could include vaccination schedules in which the multivalent recombinant MDVₙₚ of the present invention and the additional antigen are not applied simultaneously; e.g., the recombinant MDVₙₚ may be applied in ovo, and the NDV C2 and/or the IBDV strain (e.g., 89/03) could be applied at a subsequent time/date.

Accordingly, the vaccines of the present invention can be administered to the avian subject in a single dose or in multiple doses. For example, a vaccine of the present invention may be applied at the day of hatch and/or in ovo at day 16-18 (Embryonation Day) ED. When multiple doses are administered, they may be given either at the same time or sequentially, in a manner and time compatible with the formulation of the vaccine, and in such an amount as will be immunologically effective. Therefore, a vaccine of the present invention may effectively serve as a priming vaccination, which later can be followed and amplified by a booster vaccination of the identical vaccine, or with a different vaccine preparation e.g., a classical inactivated, adjuvanted whole-virus vaccine.

The volume per dose of a vaccine of the present invention can be optimized according to the intended route of application: in ovo inoculation is commonly applied with a volume between 0.05 and 0.5 ml/egg, and parenteral injection is commonly done with a volume between 0.1 and 1 ml/avian. In any case, optimization of the vaccine dose volume is well within the capabilities of the skilled artisan.

SEQUENCE TABLE

| SEQ ID | | |
|---|---|---|
| NO: | Description | Type |
| 1 | ILTV gD Glycoprotein | nucleic acid |
| 2 | ILTV gD Glycoprotein | amino acid |
| 3 | ILTV gI Glycoprotein | nucleic acid |
| 4 | ILTV gI Glycoprotein | amino acid |
| 5 | IBDV VP2 | nucleic acid |
| 6 | IBDV VP2 | amino acid |
| 7 | ILTV gD promoter | nucleic acid |
| 8 | ILTV gI promoter | nucleic acid |
| 9 | ILTV insert | nucleic acid |
| 10 | mCMV IE promoter | nucleic acid |
| 11 | chicken β-actin promoter | nucleic acid |
| 12 | hCMV IE promoter (from strain AD169) | nucleic acid |
| 13 | FHV US-9 | nucleic acid |
| | polyadenylation signal | |
| 14 | HSV TK | nucleic acid |
| | polyadenylation signal | |
| 15 | 228509-ILT-435Vec6 (HVT/IBDV/ILT 1386-134) | nucleic acid |
| | mCMV IEpro-VP2-SV40pA/ILT/HVT | |
| 16 | 1333-85.B6 (HVT/ILT/IBDV 1386-48.1.1.1) | nucleic acid |
| | ILT/Chicken β-actin pro-VP2-FHV US9pA/HVT | |
| 17 | 1386-04.4#1 (HVT/ILT/IBDV 1386-48.3.1.7) | nucleic acid |
| | ILT/hCMV IEpro-VP2-HSV TKpA/HVT | |
| 18 | 484-1050-2641-10859 (HVT/IBDV/ILT 484) | nucleic acid |
| | mCMV IEpro-VP2-SV40pA/ILT/HVT | |
| 19 | SV40 polyadenylation signal | nucleic acid |

The present invention may be better understood by reference to the following non-limiting examples, which are provided as exemplary of the invention. The following examples are presented in order to more fully illustrate embodiments of the invention and should in no way be construed as limiting the broad scope of the invention.

### EXAMPLES

### Example 1

### Construction of Recombinant HVT/ILTV/IBDV Virus Vectors

Recombinant multivalent non-pathogenic Marek's Disease virus constructs were prepared that encode and express both Infectious Laryngotracheitis Virus and Infectious Bursal Disease Virus protein antigens. The present invention overcomes the problem of vaccine interference encountered when two recombinant HVT vaccines, such as Innovax®-ILT (sold by Merck Animal Health) and Vaxxitek® (sold by Merial) are given to the same animal.

Recombinant virus constructs were created in which antigenic donor material from two poultry pathogens, Infectious Laryngotracheitis Virus (ILTV) and Infectious Bursal Disease virus (IBDV) are inserted into a Herpesvirus of Turkey (HVT) vector [see also, U.S. Pat. No. 8,932,604 B2, WO 2013/057,235, and WO 2016/102647, the contents of all of which are hereby incorporated by reference in its entireties]. The donor materials include a 3.563 kb SalI-HindIII fragment from ILTV, NVSL Challenge Strain, Lot #83-2 [pos. 10532-14094; Wild et al., Virus Genes 12:104-116(1996): Acc. #U28832], encoding the full length genes for glycoprotein D (gD) and glycoprotein I (gI), plus partial coding regions from glycoprotein E (amino acids 1-101), and ORF5 (amino acids 734-985); and an expression cassette consisting of the coding region for IBDV, Faragher, type F52/70 strain, viral protein 2 (vp2) gene, driven by a eukaryotic or viral promoter and followed by a terminator sequence. In the present embodiment, the promoter driving VP2 expression may come from the immediate early (IE) gene of human cytomegalovirus (hCMV), strain AD 169, from chicken beta-actin (cp-act) gene or from the IE gene of mouse cytomegalovirus (mCMV) strain ATCC VR-194. The terminator and polyadenylation sequence may come from human Herpes Simplex Virus (HSV), thymidine kinase (TK) gene, from the glycoprotein B (gB) gene of Feline Herpesvirus (FHV), from the immediate early (IE) gene of human cytomegalovirus (hCMV), strain AD 169 or from simian virus 40 (SV40). The donor material may be inserted into one of two non-essential sites in the HVT vector, the US2 site [pos. 140540/140541, Afonso et al., J. Virology 75(2):971-978 (2001); Acc. #AF291866, between amino acids residues 124 and 125], or in the UL54.5 site [pos. 111240/111241, Afonso et al., 2001, supra; Acc. #AF291866, between amino acids residues 21 and 22], or one insert in each site.

Genetic and phenotypic stability is a major component of the safety and relatively strong antigen expression and/or efficacy profile of any new recombinant viral vaccine candidate. The IBDV and ILTV expression cassettes inserted into the HVT backbone are not intrinsically required for viral replication and therefore may be lost due to mutation during amplification of the virus stock in tissue culture passages. A satisfactory vaccine candidate must not easily mutate to lose expression of the foreign gene insert. A vaccine candidate is considered stable if it can be demonstrated that at least 90% of the viral plaques express the inserted foreign antigenic protein following greater than or equal to 10 passages in tissue culture.

### Example 2

### Construction of Recombinant HVT/ILTV/IBDV Virus Vectors

The ability to generate herpesviruses by this method has previously been demonstrated for pseudorabies virus [van Zijl et al., J. Virology 62:2191-2195 (1988)]. This procedure subsequently was employed to construct recombinant HVT vectors [see, U.S. Pat. No. 5,853,733, hereby incorporated by references with respect to the methodology disclosed regarding the construction of recombinant HVT vectors] and was used to construct the recombinant HVT/ILTV/IBDV vectors of the present invention. In this method, the entire HVT genome is cloned into bacterial vectors as several large overlapping subgenomic fragments constructed utilizing standard recombinant DNA techniques [Maniatis et al., (1982) Molecular Cloning, Cold Spring Harbor Laboratory press, Cold Spring Harbor, New York (1982); and Sambrook et al., Molecular Cloning, Cold Spring Harbor Laboratory press, Cold Spring Harbor, New York (1989)]. An HVT strain FC126 cosmid library was derived from sheared viral DNA cloned into the cosmid vector pWE15 (Stratagene, now Agilent Technologies of Santa Clara, Calif.). In addition, several large genomic DNA fragments were isolated by restriction digestion with the enzyme, BamHI, and cloned into either pWE15 or the plasmid vector pSP64 (Promega, Madison Wis.). As described in U.S. Pat. No. 5,853,733, cotransfection of these fragments into chicken embryo fibroblast (CEF) cells results in the regeneration of HVT genome mediated by homologous recombination across the overlapping regions of the fragments. If an insertion is engineered directly into one or more of the subgenomic fragments prior to the cotransfection, this procedure results in a high frequency of viruses containing the insertion. Five overlapping subgenomic clones are required to generate HVT/FC126 HVT, and served as the basis for creating all HVT/ILTV/IBDV recombinant viruses.

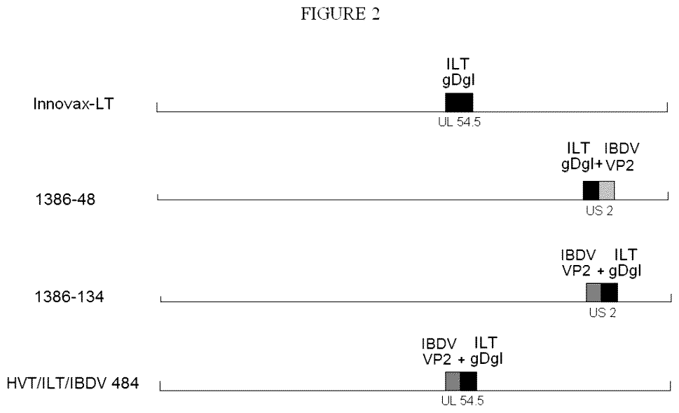

### Construction of HVT/LT/RBDV 1386-134.1-2: [See, 1386-134 Depicted in FIG. 2]

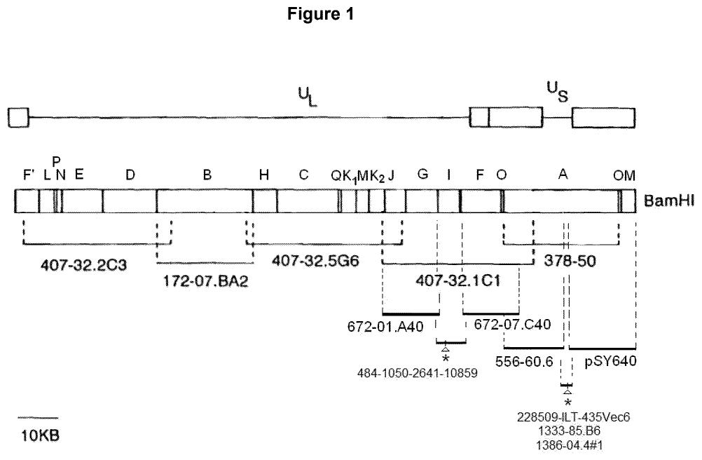

The cosmid regeneration of HVT/ILT/IBDV 1386-134.1-2 was performed essentially as described in U.S. Pat. No. 5,853,733 [e.g. FIG. 8 of U.S. Pat. No. 5,853,733; redrawn, at least in part, in FIG. 1, herein]. To allow integration into the US region of the FC126 HVT genome, the region covered by the cosmid nr. 378-50 in U.S. Pat. No. 5,853,733, was now provided from three smaller plasmids: pSY640 and 556-60.6, and one transfer plasmid (228509-ILT-435Vec6), overlapping these two, and containing the IBDV/ILTV expression cassettes in the US2 gene locus. The set of seven linearized constructs: 3 cosmids and 4 plasmids are transfected all together into CEFs, using a standard CaCl₂ transfection protocol and the resulting virus stock was plaque purified two times.

### Construction of HVT/LT/RBDV 1386-48.1.1.1: [See, 1386-48 Depicted in FIG. 2]

The cosmid regeneration of HVT/ILT/IBDV 1386-48.1.1.1 was performed essentially as described in U.S. Pat. No. 5,853,733 [e.g. FIG. 8 of U.S. Pat. No. 5,853,733; redrawn, at least in part, in FIG. 1, herein]. To allow integration into the US region of the FC126 HVT genome, the region covered by the cosmid nr. 378-50 in U.S. Pat. No. 5,853,733, was now provided from three smaller plasmids: pSY640 and 556-60.6, and one transfer plasmid (1333-85.B6), overlapping these two, and containing the IBDV/ILTV expression cassettes in the US2 gene locus.

The set of seven linearized constructs: 3 cosmids and 4 plasmids are transfected all together into CEFs, using a standard CaCl₂ transfection protocol and the resulting virus stock was plaque purified two times.

### Construction of HVT/ILT/IBDV 1386-48.3.1.7: [See, 138648 Depicted in FIG. 2]

The cosmid regeneration of HVT/ILT/IBDV 1386-48.3.1.7 was performed essentially as described in U.S. Pat. No. 5,853,733 [e.g. FIG. 8 of U.S. Pat. No. 5,853,733; redrawn, at least in part, in FIG. 1, herein]. To allow integration into the US region of the FC126 HVT genome, the region covered by the cosmid nr. 378-50 in U.S. Pat. No. 5,853,733, was now provided from three smaller plasmids: pSY640 and 556-60.6, and one transfer plasmid (1386-04.4 #1), overlapping these two, and containing the IBDV/ILTV expression cassettes in the US2 gene locus. The set of seven linearized constructs: 3 cosmids and 4 plasmids are transfected all together into CEFs, using a standard CaCl₂ transfection protocol and the resulting virus stock was plaque purified two times.

### Construction of HVT/ILT/IBDV 484: [See, 1386-484 Depicted in FIG. 2]

The cosmid regeneration of HVT/ILT/IBDV 484 was performed essentially as described in U.S. Pat. No. 5,853,733 [e.g. FIG. 8 of U.S. Pat. No. 5,853,733; redrawn, at least in part, in FIG. 1, herein]. To allow integration into the UL54.5 region of the FC126 HVT genome, the region covered by the cosmid nr. 407-32.1C1 in U.S. Pat. No. 5,853,733, was now provided from three smaller plasmids: 672-01.A40 and 672-07.C40, and one transfer plasmid (484-1050-2641-10859), overlapping these two, and containing the IBDV/ILTV expression cassettes in the UL54.5 gene locus. The set of seven linearized constructs: 4 cosmids and 3 plasmids are transfected all together into CEFs, using a standard CaCl₂ transfection protocol and the resulting virus stock was plaque purified two times.

### Description of Subgenomic Fragments for Generating FC126 HVT:

### Subgenomic Clone 407-32.2C3

Cosmid 407-32.2C3 contains an approximately 40,170 base pair region of genomic HVT DNA [Left terminus—pos. 39,754; Afonso et al., 2001, supra; Acc. #AF291866]. This region includes HVT BamHI fragments F′, L, P, N1, E, D, and 2,092 base pairs of fragment B.

### Subgenomic Clone 172-07.BA2

Plasmid 172-07.BA2 contains a 25,931 base pair region of genomic HVT DNA. It was constructed by cloning the HVT BamHI B fragment [pos. 37,663 to 63,593; Afonso et al., 2001, supra; Acc. #AF291866], into the plasmid pSP64 (Promega, Madison Wis.).

### Subgenomic Clone 407-32.5G6

Cosmid 407-32.5G6 contains a 39,404 base pair region of genomic HVT DNA [pos. 61,852-101,255; Afonso et al., 2001, supra; Acc. #AF291866]. This region includes HVT BamHI fragments H, C, Q, K1, M, K2, plus 1,742 base pairs of fragment B, and 3,880 base pairs of fragment J. Subgenomic Clone 407-31.

1C1Cosmid 407-31.1C1 contains a 37,444 base pair region of genomic HVT DNA [pos. 96,095-133,538; Afonso et al., 2001, supra; Acc. #AF291866]. This region includes HVT BamHI fragments J, G, I, F, O, plus 1,281 base pairs of fragment K2, and 6,691 base pairs of fragment A.

### Subgenomic Clone 378-50

Cosmid 378-50 contains a 28,897 base pair region of genomic HVT DNA [see, FIG. 8 of U.S. Pat. No. 5,853,733; redrawn, at least in part, in FIG. 1, herein]. This region includes HVT BamHI fragment A. It was constructed by cloning the HVT BamHI A fragment [pos. 126,848-155,744; Afonso et al., 2001, supra; Acc. #AF291866] into cosmid pWE15.

### Additional Insertion Fragments for Generating HVT/ILT/IBDV 1386-134.1-2:

### Subgenomic Clone 228059-ILT-435Vec6

The insertion plasmid 228059-ILT-435Vec6 contains a 7311 base pair EcoRI fragment of the HVT unique short regions [pos. 126880-144190; Afonso et al., 2001, supra; Acc. #AF291866], cloned into the plasmid pSP64 (Promega, Madison, WI.). Inserted into a unique StuI site within the HVT US2 gene [pos. 140540/140541; Afonso et al., 2001, supra; Acc. #AF291866, between amino acid residues 124 and 125] are 2 elements: an expression cassette consisting of the mCMV IE promoter, the IBDV classic type F52/70, Faragher strain, virus protein 2 gene (VP2), and the SV40 polyadenylation signal, followed by a 3563 base pair SalI-HindIII fragment from ILTV, NVSL Challenge Strain, Lot #83-2 [pos. 10532-14094; Wild et al., Virus Genes 12:104-116 (1996); Acc. #U28832], encoding the full length genes for glycoprotein D (gD) and glycoprotein I (gI), plus partial coding regions from glycoprotein E (amino acids 1-101), and ORF5 (amino acids 734-985). The IBDV VP2, ILTV gD and ILTV gI genes are transcribed in the opposite direction relative to the HVT US2 gene.

Subgenomic Clone pSY640

Plasmid pSY640 contains an approximately 13,600 base pair region of genomic HVT DNA (pos. 126848-140540; Afonso et al., 2001, supra; Acc. #AF291866] derived from BamHI fragment A. To generate this plasmid the region of DNA located upstream of the US2 gene, beginning at the StuI site located in the US2 gene and continuing to the end of the BamHI A fragment, was cloned into the plasmid pSP64 (Promega, Madison WI.).

### Subgenomic Clone 556-60.6

Plasmid 556-60.6 contains an approximately 12,500 base pair region of genomic HVT DNA derived from BamHI fragment A (approximate pos. 143300-155744; Afonso et al., 2001, supra; Acc. #AF291866]. To generate this plasmid, the region of DNA located downstream of the US2 gene (beginning at the StuI site located in the US2 gene and continuing to the end of the BamHI A fragment) was cloned into pSP64 (Promega, Madison WI.), and then treated with exonuclease to “chewed back” from StuI site ˜150 bp, and re-cloned into pBR322 plasmid vector.

### Additional Insertion Fragments for Generating HVT/ILT/IBDV 1386-48.1.1.1:

### Subgenomic Clone 1333-85.B6

The insertion plasmid 1333-85.B6 contains a 7311 base pair EcoRI fragment of the HVT unique short regions [pos. 126880-144190; Afonso et al., 2001, supra; Acc. #AF291866], cloned into the plasmid pSP64 (Promega, Madison, WI.). Inserted into a unique StuI site within the HVT US2 gene [pos. 140540/140541; Afonso et al., 2001, supra; Acc. #AF291866, between amino acid residues 124 and 125] are 2 elements: a 3563 base pair SalI-HindIII fragment from ILTV, NVSL Challenge Strain, Lot #83-2 [pos. 10532-14094; Wild et al., Virus Genes 12:104-116 (1996); Acc. #U28832], encoding the full length genes for glycoprotein D (gD) and glycoprotein I (gI), plus partial coding regions from glycoprotein E (amino acids 1-101), and ORF5 (amino acids 734-985) and an expression cassette consisting of the chicken β-Actin promoter, the IBDV classic type F52/70, Faragher strain, virus protein 2 gene (VP2), and the polyadenylation signal from the Feline Herpesvirus (FHV) glycoprotein B gene. The ILTV gD, ILTV gI and IBDV VP2 genes are transcribed in the opposite direction relative to the HVT US2 gene.

Subgenomic Clone pSY640.

Plasmid pSY640 contains an approximately 13,600 base pair region of genomic HVT DNA (pos. 126848-140540; Afonso et al., 2001, supra; Acc. #AF291866] derived from BamHI fragment A. To generate this plasmid the region of DNA located upstream of the US2 gene, beginning at the StuI site located in the US2 gene and continuing to the end of the BamHI A fragment, was cloned into the plasmid pSP64 (Promega, Madison WI.).

### Subgenomic Clone 556-60.6.

Plasmid 556-60.6 contains an approximately 12,500 base pair region of genomic HVT DNA derived from BamHI fragment A (approximate pos. 143300-155744; Afonso et al., 2001, supra; Acc. #AF291866]. To generate this plasmid, the region of DNA located downstream of the US2 gene (beginning at the StuI site located in the US2 gene and continuing to the end of the BamHI A fragment) was cloned into pSP64 (Promega, Madison WI.), and then treated with exonuclease to “chewed back” from StuI site ˜150 bp, and re-cloned into pBR322 plasmid vector.

### Additional Insertion Fragments for Generating HVT/ILT/IBDV 1386-48.3.1.7:

### Subgenomic Clone 1386-04.4 #1

The insertion plasmid 1386-04.4 #1 contains a 7311 base pair EcoRI fragment of the HVT unique short regions [pos. 126880-144190; Afonso et al., 2001, supra; Acc. #AF291866], cloned into the plasmid pSP64 (Promega, Madison, WI.). Inserted into a unique StuI site within the HVT US2 gene [pos. 140540/140541; Afonso et al., 2001, supra; Acc. #AF291866, between amino acid residues 124 and 125] are 2 elements: a 3563 base pair SalI-HindIII fragment from ILTV, NVSL Challenge Strain, Lot #83-2 [pos. 10532-14094; Wild et al., Virus Genes 12:104-116 (1996); Acc. #U28832], encoding the full length genes for glycoprotein D (gD) and glycoprotein I (gI), plus partial coding regions from glycoprotein E (amino acids 1-101), and ORF5 (amino acids 734-985) and an expression cassette consisting of the hCMV IE promoter, the IBDV classic type F52/70, Faragher strain, virus protein 2 gene (VP2), and the polyadenylation signal from the Herpes Simplex virus (HSV) thymidine kinase gene. The ILTV gD, ILTV gI, and IBDV VP2 genes are transcribed in the opposite direction relative to the HVT US2 gene.

Subgenomic Clone pSY640.

Plasmid pSY640 contains an approximately 13,600 base pair region of genomic HVT DNA (pos. 126848-140540; Afonso et al., 2001, supra; Acc. #AF291866] derived from BamHI fragment A. To generate this plasmid the region of DNA located upstream of the US2 gene, beginning at the StuI site located in the US2 gene and continuing to the end of the BamHI A fragment, was cloned into the plasmid pSP64 (Promega, Madison WI.).

### Subgenomic Clone 556-60.6.

Plasmid 556-60.6 contains an approximately 12,500 base pair region of genomic HVT DNA derived from BamHI fragment A (approximate pos. 143300-155744; Afonso et al., 2001, supra; Acc. #AF291866]. To generate this plasmid, the region of DNA located downstream of the US2 gene (beginning at the StuI site located in the US2 gene and continuing to the end of the BamHI A fragment) was cloned into pSP64 (Promega, Madison WI.), and then treated with exonuclease to “chewed back” from StuI site ˜150 bp, and re-cloned into pBR322 plasmid vector.

### Additional Insertion Fragments for Generating HVT/ILT/IBDV 484:

### Subgenomic Clone 484-1050-2641-10859

The insertion plasmid 484-1050-2641-10859 contains a 8636 base pair region of genomic HVT unique long region [pos. 109489-118124; Afonso et al., 2001, supra; Acc. #AF291866], cloned into a derivative of plasmid pNEB193 (deleted AatII-PvuII). It is flanked by AscI sites and includes HVT BamHI fragments I, S, plus 1337 base pairs of fragment G and 1177 base pairs of fragment F. Inserted into an XhoI site within the HVT UL54.5 open reading frame [pos. 111240/111241; Afonso et al., 2001, supra; Acc. #AF291866, between amino acid residues 21 and 22] are 2 elements: an expression cassette consisting of the mCMV IE promoter, the IBDV classic type F52/70, Faragher strain, virus protein 2 gene (VP2), and the SV40 polyadenylation signal, followed by a 3563 base pair SalI-HindIII fragment from ILTV, NVSL Challenge Strain, Lot #83-2 [pos. 10532-14094; Wild et al., Virus Genes 12:104-116 (1996); Acc. #U28832], encoding the full length genes for glycoprotein D (gD) and glycoprotein I (gI), plus partial coding regions from glycoprotein E (amino acids 1-101), and ORF5 (amino acids 734-985). The IBDV VP2, ILTV gD, and ILTV gI genes are transcribed in the opposite direction relative to the HVT UL54.5 gene.

### Subgenomic Clone 672-01.A40

Plasmid 672-01.A40 contains a 14,731 base pair region of genomic HVT DNA derived from the unique long region [pos. 96095-110825; Afonso et al., 2001, supra; Acc. #AF291866], cloned into a derivative of plasmid pNEB193. This region includes HVT BamHI fragments G, J and 1281 base pairs of K2.

Subgenomic clone 672-07.C40

Plasmid 672-07.C40 contains a 12,520 base pair region of genomic HVT DNA derived from the unique long region [pos. 116948-129467; Afonso et al., 2001, supra; Acc. #AF291866], cloned into a derivative of plasmid pNEB193. This region includes HVT BamHI fragments F, O and 2620 base pairs of A.

### Standard CaCl₂ Transfection Protocol:

Secondary CEF's are seeded on 6 well culture plates and incubated at 38° C. with 5% CO₂ for 24 hours and confluent monolayers form. For each well a total amount of 0.5 μg DNA of cosmids and plasmids were mixed in Hepes buffer and 125 mM CaCl₂ was added dropwise until precipitation was imminent. This mixture was added to the CEF cell monolayer, and incubated for 2 to 3 hours. Supernatant was removed and an overlay of 15% Glycerol was added, and kept on the cells for 1 minute. Then this was removed, washed with PBS, and fresh culture medium was added and cells were incubated for 5 days. Next, cells were harvested by trypsinization and cells from individual plates were each seeded on fresh monolayers of CEF cells in 10 cm plates and incubated until 50-90% CPE was achieved. Next, the amplified transfected cells were harvested by trypsinization, and dilutions of 10⁻² to 10⁻⁴ were plated on 10 cm plates with CEF monolayers and incubated. The following day, the plates were covered with agar, and a number of individual plaques of HVT/ILTV/IBDV were isolated and amplified on CEFs. Each virus stock was plaque purified a second time by infecting confluent monolayers of CEFs on 10 cm plates with first round purified stocks diluted to 10⁻² to 10⁻⁴ and incubating cells. The following day, the plates were covered with agar, and a number of individual plaques of HVT/ILTV/IBDV were isolated and amplified on CEFs.

### Example 3

### Recombinant HVT/ILTV/IBDV Virus Stocks are Phenotypically Stable for Expression of the ILT and IBDV Proteins Following Serial Passage in Tissue Culture

Three constructs, one comprising HVT/IBDV/ILT 1386-134.1-2, the second, comprising HVT/ILT/IBDV 1386-48.1.1.1, and the third comprising HVT/ILT/IBDV 1386-48.3.1.7 were serial passaged greater than 14 times on secondary CEF cells and evaluated for expression of the inserted ILTV and IBDV genes in an Immunofluorescence Assay. A fourth construct, designated HVT/ILT/IBDV 484.1-1A3A3 was serial passaged greater than 15 times on secondary CEF cells and evaluated for expression of the inserted ILTV and IBDV genes in an Immunofluorescence Assay, [see, Tables 1 and 2 below].

### Generation of Tissue Culture Passage Stocks:

For each tissue culture passage, confluent secondary CEF monolayers, plated on a 10 cm dish were inoculated with 50-100 L of virus stock, then incubated at 38° C., 5% CO₂ for 2-5 days until CPE was evident. Next, cells were harvested by trypsinization, passage 1 (P1). The process was repeated to prepare further passage stocks (P2-P15).

### Phenotypic Stability Analysis:

Six well plates were planted with secondary CEF monolayers. The cells were inoculated with virus stocks harvested at various passage levels: P0-P15, or diluent alone. Plates were inoculated at multiple dilutions to achieve a countable number of plaques per well, and incubated at 38° C., 5% CO₂. After a five day incubation, supernatant was decanted and CEF monolayers were fixed with 100% methanol for 10-15 minutes at 15-30° C. Methanol was decanted and cells allowed to air dry prior to staining with ILTV gD (MAB #6), ILTV gI (polyclonal Rabbit anti-gI), and IBDV VP2 (MCA GDV-R63) primary antibodies. Following a 2 hour blocking step, (5% non-fat dry milk in PBS), 2 mL per well, was added to dishes, and incubated on a rocking platform at 15-30° C., primary antibodies were diluted as appropriate and added at 2 mL per well, then incubated at 15-30° C. for 3 hours on a rocking platform. After antibody incubation, plates were washed three times with PBS. The FITC-labeled secondary antibody solution (Rabbit anti-mouse or Goat anti-rabbit) was prepared at 1:100 and 2 mL was added to each well. Plates were incubated for 1 hour at 15-30° C. on a rocking platform. Following the incubation, the plates were washed three times with PBS, and examined under a fluorescent scope. Plaques stained with the ILT antibodies were observed for positive (+) or negative (−) fluorescence. Fluorescing plaques stained with primary antibody to IBDV VP2 protein were counted. Plates were then examined under a white light microscope and the plaques were re-counted. The percentage of fluorescing plaques at each passage level is provided in Tables 1 and 2 below, as well as a qualitative determination of the IBDV VP2 protein expression and vector stability.

TABLE 1
STABILITY OF EXPRESSION FOLLOWING PASSAGE IN
TISSUE CULTURE

| | | | | Expression | | |
|---|---|---|---|---|---|---|
| Virus | | Insertion | Passage | ILT | ILT | IBDV |
| Number | Description | site | Level | gD | gI | VP2 |
| 1386-48.3.1.7 | (h)IE-VP2/ | US2 | P0 | + | + | 100% |
| | ILTgDgI | | P5 | + | + | 100% |
| | | | P9 | + | + | 100% |
| | | | P14 | + | + | 96% |
| 1386-48.1.1.1 | (c)β-act-VP2/ | US2 | P0 | + | + | 100% |
| | ILTgDgI | | P5 | + | + | 100% |
| | | | P10 | + | + | 97% |
| | | | P15 | + | + | 98% |
| 1386-134.1-2 | (m)IE-VP2/ | US2 | P0 | + | + | 100% |
| | ILTgDgI | | P5 | + | + | 100% |
| | | | P10 | + | + | 98% |
| | | | P15 | + | + | 99% |
| 484.1-1A3A3 | (m)IE-VP2/ | UL54.5 | P0 | + | + | 100% |
| | ILTgDgI | | P4 | + | + | 100% |
| | | | P10 | + | + | 97% |
| | | | P15 | + | + | 94% |

TABLE 2
INSERTION PLASMID DESCRIPTION/VECTOR PROPERITES

| | | | | IBDV | |
|---|---|---|---|---|---|
| Name/ | Insert. | Insert. | IBDV | Expres- | Stabil- |
| Designation | site | Plasmid | Promoter | sion | ity |
| HVT/ILT/IBDV | US2 | 1333-85.B6 | chicken | Strong | Stable |
| 1386-48.1.1.1 | | | β-actin | | |
| HVT/ILT/IBDV | US2 | 1386-04.4#1 | hCMV IE | Strong | Stable |
| 1386-48.3.1.7 | | | | | |
| HVT/IBDV/ILT | US2 | 228509-ILT- | mCMV IE | Strong | Stable |
| 1386-134.1-2 | | 435Vec6 | | | |
| HVT/IBDV/ILT | UL54.5 | 484-1050- | mCMV IE | Strong | Stable |
| 484 | | 2641-10859 | | | |

### Example 4

Recombinant HVT/ILTV/IBDV Virus Stocks are Phonetically Stable for Expression of the ILT and IBDV Proteins Following Vaccination and Recovery from Birds

Three vaccines, one comprising HVT/ILT/IBDV 1386-134.1-2, another comprising HVT/ILT IBDV 1386-48.3.1.7, and a third comprising HVT/ILT/IBDV 484.1-1A3A3 were used to inoculate three groups of fifteen (15) day-of-age chickens by the subcutantious route. A fourth group of birds were vaccinated with diluent alone to serve as a negative control group. Spleen samples were collected seven days post-inoculation, and processed for virus isolation on chicken embryo fibroblast cells. Inoculated cells were passaged two times to allow expansion of any virus present. When cytopathic effect was clearly visible, monolayers were harvested and stock frozen. These stocks were used to inoculate secondary CEFs, and plaques analyzed for expression of the ILTV gD, ILTV gI, and IBDV VP2 proteins by immunofluorescence assay (IFA) assay, with antibodies specific to each protein.

### Phenotypic Stability Analysis

Six well plates were planted with secondary CEF monolayers. The cells were inoculated with the harvested virus isolation stocks or diluent alone. The plates were inoculated at multiple dilutions to achieve a countable number of plaques per well, and incubated at 38° C., 5% CO₂. After five days incubation, supernatant was decanted and CEF monolayers were fixed with 100% methanol for 10-15 minutes at 15-30° C. Methanol was decanted and cells allowed to air dry prior to staining with ILTV gD (MAB #6), ILTV gI (polyclonal Rabbit anti-gI), and IBDV VP2 (MCA GDV-R63) primary antibodies. Following a 2 hour blocking step, (5% non-fat dry milk in PBS), 2 mL per well, was added to dishes, and incubated on a rocking platform at 15-30° C., primary antibodies were diluted as appropriate, and added at 2 mL per well, then incubated at 15-30° C. for 3 hours on a rocking platform. After antibody incubation, plates were washed three times with PBS. The FITC-labeled secondary antibody solution (Rabbit anti-mouse or Goat anti-rabbit) was prepared at 1:100 and 2 mL was added to each well. Plates were incubated for 1 hour at 15-30° C. on a rocking platform. Following incubation, plates were washed three times with PBS, and examined under a fluorescent scope. Plaques stained with the ILTV antibodies were observed for positive (+) or negative (−) fluorescence. Fluorescing plaques stained with primary antibody to IBDV VP2 were counted. Plates were then examined under a white light microscope and plaques re-counted. The percentage of fluorescing plaques at each passage level is provided in Table 3A below. This study was essentially repeated except the virus was recovered two weeks post-inoculation, see, Table 3B below.

TABLE 3A
STABILITY OF EXPRESSION FOLLOWING PASSAGE IN BIRDS

| | | | | Percent Expressing | | |
|---|---|---|---|---|---|---|
| | Insert | Insertion | Dose | | ILT | IBDV |
| Vaccine | Description | site | (PFU) | ILT gI | gD | VP2 |
| HVT/ILT/IBDV | (m)IE-VP2/ | US2 | 7737 | 100% | 100% | 90% |
| 1386-134.1-2 | ILTgDgI | | | | | |
| (p10) | | | | | | |
| HVT/ILT/IBDV | (h)IE-VP2/ | US2 | 7003 | 100% | 100% | 89% |
| 1386-48.3.1.7 | ILTgDgI | | | | | |
| (p10) | | | | | | |
| HVT/IBDV/ILT | (m)IE-VP2/ | UL54.5 | 7793 | 100% | 100% | 97% |
| 484.1-1A3A3 | ILTgDgI | | | | | |
| (p10) | | | | | | |
| Diluent | NA | NA | 0 | NA | NA | NA |

TABLE 3B
VIRUS RECOVERED 2 WEEKS POST-INOCULATION

| | | | | Percent Expressing | | |
|---|---|---|---|---|---|---|
| | Insert | Insertion | Dose | | | IBDV |
| Vaccine | Description | site | (PFU) | ILT gI | ILT gD | VP2 |
| HVT/ILT/IBDV | (h)IE-VP2/ | US2 | 4785 | 100% | 92% | 42% |
| 1386-48.1.1.1 | ILTgDgI | | | | | |
| (p10) | | | | | | |

### Example 5

### Unsuccessful Constructs

The recombinant vector vaccine viruses, by definition are engineered to carry and express foreign genes. Should transcription and expression of these foreign genes provide a growth disadvantage to the recombinant virus relative to the parental virus, it is possible for these genes to be lost during production of the vaccine. For this reason, vaccine candidates must be tested for both genetic and phenotypic stability.

In addition, the protection criteria used is that which has been established by the USDA and codified in the Title 9 Code of Federal Regulations, part 113 (9CFR 113) «Standard requirements for Animal Products». Live virus vaccines must provide at least 90% protection, in the case of NDV, IBDV and ILTV, and at least 80% in the case of MDV, from clinical signs or lesions associated with the disease to obtain a license.

Genetic stability of the viral constructs was determined by Southern blot analysis after a defined number of passages in tissue culture, the highest anticipated vaccine production level, and compared with DNA from the original isolate. DNA extracted from viral stocks would be digested with restriction enzymes, transferred to a membrane and hybridized with probes designed to detect the presence of the inserted foreign genes. Genetic stability may also be determined by PCR analysis. PCR primers designed to anneal to DNA within or flanking the foreign DNA could be used to amplify fragments of a known size from the viral DNA templates both before and after passage in tissue culture.

Phenotypic stability of the viral constructs was determined by immunological staining of individual viral plaques with antibodies directed against the protein products of these inserted foreign genes. Protection provided by these recombinant vaccines relies on expression of these protein products in order to stimulate the animals immune system. In most cases, if the percent of viruses staining positive for the foreign protein expression dropped below 90%, it was likely detrimental to the viruses ability to be grown in tissue culture, and therefore unsuitable as a vaccine candidate.

As is readily apparent from Tables 4A and 4B below, most rMDVnp constructs do not meet these two criteria, namely stability with relatively strong antigen expression and/or efficacy. Table 4A provides a series of recombinant HVT constructs with multiple heterologous inserts in which one of the heterologous inserts encodes an IBDV antigen. As the results show, all of the constructs in Table 4A failed to meet the stability with relatively strong antigen expression and/or efficacy criteria.

TABLE 4A
DOUBLE RECOMBINANT HVT AND IBDV VIRUS CONSTRUCTS:

| Name/ | Insertion | | IBDV | IBDV | |
|---|---|---|---|---|---|
| Designation | site | Insert | Promoter | Expression | Stability |
| HVT 003 | UL43 | [IBDV] polyprotein | PRV gX | Poor | stable |
| | | [Ecoli] Bgal | | | |
| HVT 016 | UL43 | [IBDV] VP2 | hCMV IE | Strong | unstable |
| | | [Ecoli] Bgal | | | |
| HVT 056 | US2 | [MDV] gA, gB | hCMV IE | Strong | Unstable |
| | | [IBDV] VP2 | | | |
| HVT 060 | US2 | [MDV] gA, gB | IE-VP2, | Strong | unstable |
| | | [IBDV] VP2, | gX-16dk | | |
| | | 16 kD ORF | ORF | | |
| HVT 137 | US2 | [MDV] gA, gB, gC | [BHV] VP8 | Poor | stable |
| | UL54.5 | [IBDV] VP2 | (tegument) | | |
| HVT 143 | US2 | [MDV] gA, gB, gD | [BHV] VP8 | Poor | Unstable |
| | US2 | [NDV] HN, F | (tegument) | | |
| | UL54.5 | [IBDV] VP2 | | | |
| HVT/NDV/IBDV | US2 | [IBDV] VP2 | hCMV IE | Strong | Unstable |
| 1312-92 | UL7/UL8 | [NDV] F | | | |
| HVT/NDV/IBDV | US2 | [IBDV] VP2 | hCMV IE | Strong | Unstable |
| 1312-94 | UL7/UL8 | [NDV] F | | | |
| HVT/NDV/IBDV | US2 | [IBDV] VP2 | hCMV IE | Strong | Unstable |
| 1312-95 | UL7/UL8 | [NDV] F | | | |
| HVT/NDV/IBDV | US2 | [IBDV] VP2 | FHV gB | Strong | Unstable |
| 1329-54 | | [NDV] F | | | |

Table 4B below, provides a series of eleven recombinant HVT constructs and one lone NAHV construct each of which comprise multiple heterologous inserts in which at least one of the heterologous inserts encodes either an NDV or an ILTV antigen.¹ As the results show, all of the constructs in Table 4B failed to meet the stability with relatively strong antigen expression and/or efficacy criteria. ¹The data in Table 4B was submitted to the U.S. Patent Office during the prosecution of U.S. Pat. No. 8,932,604 B2 in a Declaration signed by one of the co-Inventors of the present application.

TABLE 4B
DOUBLE RECOMBINANT HVT AND NAHV VIRUS CONSTRUCTS:

| | Insertion | | | NDV | MDV | ILT |
|---|---|---|---|---|---|---|
| Name | site | Insert | Stability | Protection | Protection | Protection |
| HVT 048 | US2 | [MDV] gA, gB | Stable | Good | *Protective | — |
| | | [NDV] F | | | | |
| HVT 049 | US2 | [MDV] gA, gB | Stable | Poor | Not tested | — |
| | | [NDV] HN | | (<20%) | | |
| HVT 050 | US2 | [MDV] gA, gB | Stable | Good | *Protective | — |
| | | [NDV] F, HN | | | | |
| HVT 053 | US2 | [MDV] gA, gB | Unstable | — | Not tested | None |
| | | [ILT] gB, gD | | | | |
| HVT 078 | US2 | [MDV] gA, gB, gD | Unstable | Not tested | Not tested | — |
| | | [NDV]HN, F | | | | |
| HVT 079 | US2 | [MDV] gA, gB, gD | Unstable | — | Not tested | (71-100%) |
| | | [ILT] gB, gD | | | | |
| HVT 106 | US2 | [MDV]gA, gB, gD | Stable | **Unknown | Not tested | — |
| | | [NDV]HN, F | | | | |
| HVT 123 | UL54.5 + | [ILT] gD, gB/UL54.5 | Unstable | — | Not tested | Not tested |
| | US2 | [MDV] gA, gD, gB/US2 | | | | |
| HVT 125 | UL54.5 + | [ILT] gDgI, gB/UL54.5 | Unstable | — | Not tested | Not tested |
| | US2 | [MDV] gA, gD, gB/US2 | | | | |
| HVT 128 | UL54.5 + | [NDV] HN, F/UL54.5 | Unstable | Not tested | Not tested | — |
| | US2 | [MDV] gA, gD, gB/US2 | | | | |
| HVT 139 | UL54.5 + | [ILT] gDgI/UL54.5 | Unstable | — | Not tested | Not tested |
| | US2 | [MDV] gA, gD, gB/US2 | | | | |
| HVY-198 | US2* | [NDV] F + | Unstable | | | |
| (NAHV) | (MDV) | [ILT] gD, gI | | | | |

*Protective, but subsequently failed in field studies
**Only 75% birds seroconverted to NDV F

### Example 6

### Sequences

The following sequences have been used in the exemplary rHVT constructs. The coding sequences provided below include individual stop codons, which can be readily replaced with alternative stop codons without modifying the properties of the protein antigens that the coding sequences encode.

SEQ ID NO 1: ILTV gD Glycoprotein
(1134 bp)
atggaccgccatttatttttgaggaatgctttttg
gactatcgtactgctttcttccttcgctagccaga
gcaccgccgccgtcacgtacgactacattttaggc
cgtcgcgcgctcgacgcgctaaccataccggcggt
tggcccgtataacagatacctcactagggtatcaa
gaggctgcgacgttgtcgagctcaacccgatttct
aacgtggacgacatgatatcggcggccaaagaaaa
agagaaggggggccctttcgaggcctccgtcgtct
ggttctacgtgattaagggcgacgacggcgaggac
aagtactgtccaatctatagaaaagagtacaggga
atgtggcgacgtacaactgctatctgaatgcgccg
ttcaatctgcacagatgtgggcagtggactatgtt
cctagcacccttgtatcgcgaaatggcgcgggact
gactatattctcccccactgctgcgctctctggcc
aatacttgctgaccctgaaaatcgggagatttgcg
caaacagctctcgtaactctagaagttaacgatcg
ctgtttaaagatcgggtcgcagcttaactttttac
cgtcgaaatgctggacaacagaacagtatcagact
ggatttcaaggcgaacacctttatccgatcgcaga
caccaatacacgacacgcggacgacgtatatcggg
gatacgaagatattctgcagcgctggaataatttg
ctgaggaaaaagaatcctagcgcgccagaccctcg
tccagatagcgtcccgcaagaaattcccgctgtaa
ccaagaaagcggaagggcgcaccccggacgcagaa
agcagcgaaaagaaggcccctccagaagactcgga
ggacgacatgcaggcagaggcttctggagaaaatc
ctgccgccctccccgaagacgacgaagtccccgag
gacaccgagcacgatgatccaaactcggatcctga
ctattacaatgacatgcccgccgtgatcccggtgg
aggagactactaaaagttctaatgccgtctccatg
cccatattcgcggcgttcgtagcctgcgcggtcgc
gctcgtggggctactggtttggagcatcgtaaaat
gcgcgcgtagctaa
SEQ ID NO 2: ILTV gD Glycoprotein
(377 amino acids)
MDRHLFLRNAFWTIVLLSSFASQSTAAVTYDYILG
RRALDALTIPAVGPYNRYLTRVSRGCDVVELNPIS
NVDDMISAAKEKEKGGPFEASVVWFYVIKGDDGED
KYCPIYRKEYRECGDVQLLSECAVQSAQMWAVDYV
PSTLVSRNGAGLTIFSPTAALSGQYLLTLKIGRFA
QTALVTLEVNDRCLKIGSQLNFLPSKCWTTEQYQT
GFQGEHLYPIADTNTRHADDVYRGYEDILQRWNNL
LRKKNPSAPDPRPDSVPQEIPAVTKKAEGRTPDAE
SSEKKAPPEDSEDDMQAEASGENPAALPEDDEVPE
DTEHDDPNSDPDYYNDMPAVIPVEETTKSSNAVSM
PIFAAFVACAVALVGLLVWSIVKCARS
SEQ ID NO 3: ILTV gl Glycoprotein
(1089 bp)
atggcatcgctacttggaactctggctctccttgc
cgcgacgctcgcacccttcggcgcgatgggaatcg
tgatcactggaaatcacgtctccgccaggattgac
gacgatcacatcgtgatcgtcgcgcctcgccccga
agctacaattcaactgcagctatttttcatgcctg
gccagagaccccacaaaccctactcaggaaccgtc
cgcgtcgcgtttcggtctgatataacaaaccagtg
ctaccaggaacttagcgaggagcgctttgaaaatt
gcactcatcgatcgtcttctgtttttgtcggctgt
aaagtgaccgagtacacgttctccgcctcgaacag
actaaccggacctccacacccgtttaagctcacta
tacgaaatcctcgtccgaacgacagcgggatgttc
tacgtaattgttcggctagacgacaccaaagaacc
cattgacgtcttcgcgatccaactatcggtgtatc
aattcgcgaacaccgccgcgactcgcggactctat
tccaaggcttcgtgtcgcaccttcggattacctac
cgtccaacttgaggcctatctcaggaccgaggaaa
gttggcgcaactggcaagcgtacgttgccacggag
gccacgacgaccagcgccgaggcgacaaccccgac
gcccgtcactgcaaccagcgcctccgaacttgaag
cggaacactttacctttccctggctagaaaatggc
gtggatcattacgaaccgacacccgcaaacgaaaa
ttcaaacgttactgtccgtctcgggacaatgagcc
ctacgctaattggggtaaccgtggctgccgtcgtg
agcgcaacgatcggcctcgtcattgtaatttccat
cgtcaccagaaacatgtgcaccccgcaccgaaaat
tagacacggtctcgcaagacgacgaagaacgttcc
caaactagaagggaatcgcgaaaatttggacccat
ggttgcgtgcgaaataaacaagggggctgaccagg
atagtgaacttgtggaactggttgcgattgttaac
ccgtctgcgctaagctcgcccgactcaataaaaat
gtga
SEQ ID NO 4: ILTV gl Glycoprotein
(362 amino acids)
MASLLGTLALLAATLAPFGAMGIVITGNHVSARID
DDHIVIVAPRPEATIQLQLFFMPGQRPHKPYSGTV
RVAFRSDITNQCYQELSEERFENCTHRSSSVFVGC
KVTEYTFSASNRLTGPPHPFKLTIRNPRPNDSGMF
YVIVRLDDTKEPIDVFAIQLSVYQFANTAATRGLY
SKASCRTFGLPTVQLEAYLRTEESWRNWQAYVATE
ATTTSAEATTPTPVTATSASELEAEHFTFPWLENG
VDHYEPTPANENSNVTVRLGTMSPTLIGVTVAAVV
SATIGLVIVISIVTRNMCTPHRKLDTVSQDDEERS
QTRRESRKFGPMVACEINKGADQDSELVELVAIVN
PSALSSPDSIKM
SEQ ID NO 5: IBDV VP2 (1362 bp)
atgacaaacctgcaagatcaaacccaacagattgt
tccgttcatacggagccttctgatgccaacaaccg
gaccggcgtccattccggacgacaccctggagaag
cacactctcaggtcagagacctcgacctacaattt
gactgtgggggacacagggtcagggctaattgtct
ttttccctggattccctggctcaattgtgggtgct
cactacacactgcagagcaatgggaactacaagtt
cgatcagatgctcctgactgcccagaacctaccgg
ccagctacaactactgcagactagtgagtcggagt
ctcacagtgaggtcaagcacactccctggtggcgt
ttatgcactaaacggcaccataaacgccgtgacct
tccaaggaagcctgagtgaactgacagatgttagc
tacaatgggttgatgtctgcaacagccaacatcaa
cgacaaaattgggaatgtcctggtaggggaagggg
tcactgtcctcagcctacccacatcatatgatctt
gggtatgtgaggcttggtgaccccattcccgctat
agggcttgacccaaaaatggtagctacatgcgaca
gcagtgacaggcccagagtctacaccataactgca
gccgatgattaccaattctcatcacagtaccaacc
aggtggggtaacaatcacactgttctcagccaaca
ttgatgctatcacaagcctcagcattgggggagag
ctcgtgtttcaaacaagcgtccaaggccttgtact
gggcgccaccatctaccttataggctttgatggga
ctgcggtaatcaccagagctgtggccgcagataat
gggctgacggccggcaccgacaatcttatgccatt
caatcttgtcattccaaccaatgagataacccagc
caatcacatccatcaaactggagatagtgacctcc
aaaagtggtggtcaggcaggggatcagatgtcatg
gtcggcaagtgggagcctagcagtgacgatccatg
gtggcaactatccaggggccctccgtcccgtcaca
ctagtagcctacgaaagagtggcaacaggatccgt
cgttacggtcgctggggtgagtaacttcgagctga
ttccaaatcctgaactagcaaagaacctggttaca
gaatacggccgatttgacccaggagccatgaacta
cacaaaattgatactgagtgagagggaccgtcttg
gcatcaagaccgtctggccaacaagggagtacact
gattttcgtgagtacttcatggaggtggccgacct
caactctcccctgaagattgcaggagcatttggct
tcaaagacataatccgggctataaggaggtaa
SEQ ID NO 6: IBDV VP2
(453 amino acids)
MTNLQDQTQQIVPFIRSLLMPTTGPASIPDDTLEK
HTLRSETSTYNLTVGDTGSGLIVFFPGFPGSIVGA
HYTLQSNGNYKFDQMLLTAQNLPASYNYCRLVSRS
LTVRSSTLPGGVYALNGTINAVTFQGSLSELTDVS
YNGLMSATANINDKIGNVLVGEGVTVLSLPTSYDL
GYVRLGDPIPAIGLDPKMVATCDSSDRPRVYTITA
ADDYQFSSQYQPGGVTITLFSANIDAITSLSIGGE
LVFQTSVQGLVLGATIYLIGFDGTAVITRAVAADN
GLTAGTDNLMPFNLVIPTNEITQPITSIKLEIVTS
KSGGQAGDQMSWSASGSLAVTIHGGNYPGALRPVT
LVAYERVATGSVVTVAGVSNFELIPNPELAKNLVT
EYGRFDPGAMNYTKLILSERDRLGIKTVWPTREYT
DFREYFMEVADLNSPLKIAGAFGFKDIIRAIRR
SEQ ID NO 7: ILTV gD
promoter (527 bp)
aaacagctgtactacagagtaaccgatggaagaac
atcggtccagctaatgtgcctgtcgtgcacgagcc
attctccggaaccttactgtcttttcgacacgtct
cttatagcgagggaaaaagatatcgcgccagagtt
atactttacctctgatccgcaaacggcatactgca
caataactctgccgtccggcgttgttccgagattc
gaatggagccttaataatgtttcactgccggaata
tttgacggccacgaccgttgtttcgcataccgctg
gccaaagtacagtgtggaagagcagcgcgagagca
ggcgaggcgtggatttctggccggggaggcaatat
atacgaatgcaccgtcctcatctcagacggcactc
gcgttactacgcgaaaggagaggtgcttaacaaac
acatggattgcggtggaaaacggtgctgctcaggc
gcagctgtattcactcttttctggacttgtgtcag
gattatgcgggagcatatctgctttgtacgcaacg
ct
SEQ ID NO 8: ILTV gl promoter (264 bp)
tgactattacaatgacatgcccgccgtgatcccgg
tggaggagactactaaaagttctaatgccgtctcc
atgcccatattcgcggcgttcgtagcctgcgcggt
cgcgctcgtggggctactggtttggagcatcgtaa
aatgcgcgcgtagctaatcgagcctagaataggtg
gtttcttcctacatgccacgcctcacgctcataat
ataaatcacatggaatagcataccaatgcctattc
attgggacgttcgaaaagc
SEQ ID NO 9: ILTV insert (3563 bp)
tcgacggcagagtcgcagacgcccctattggacgt
caaaattgtagaggtgaagttttcaaacgatggcg
aagtaacggcgacttgcgtttccaccgtcaaatct
ccctatagggtagaaactaattggaaagtagacct
cgtagatgtaatggatgaaatttctgggaacagtc
ccgccggggtttttaacagtaatgagaaatggcag
aaacagctgtactacagagtaaccgatggaagaac
atcggtccagctaatgtgcctgtcgtgcacgagcc
attctccggaaccttactgtcttttcgacacgtct
cttatagcgagggaaaaagatatcgcgccagagtt
atactttacctctgatccgcaaacggcatactgca
caataactctgccgtccggcgttgttccgagattc
gaatggagccttaataatgtttcactgccggaata
tttgacggccacgaccgttgtttcgcataccgctg
gccaaagtacagtgtggaagagcagcgcgagagca
ggcgaggcgtggatttctggccggggaggcaatat
atacgaatgcaccgtcctcatctcagacggcactc
gcgttactacgcgaaaggagaggtgcttaacaaac
acatggattgcggtggaaaacggtgctgctcaggc
gcagctgtattcactcttttctggacttgtgtcag
gattatgcgggagcatatctgctttgtacgcaacg
ctatggaccgccatttatttttgaggaatgctttt
tggactatcgtactgctttcttccttcgctagcca
gagcaccgccgccgtcacgtacgactacattttag
gccgtcgcgcgctcgacgcgctaaccataccggcg
gttggcccgtataacagatacctcactagggtatc
aagaggctgcgacgttgtcgagctcaacccgattt
ctaacgtggacgacatgatatcggcggccaaagaa
aaagagaaggggggccctttcgaggcctccgtcgt
ctggttctacgtgattaagggcgacgacggcgagg
acaagtactgtccaatctatagaaaagagtacagg
gaatgtggcgacgtacaactgctatctgaatgcgc
cgttcaatctgcacagatgtgggcagtggactatg
ttcctagcacccttgtatcgcgaaatggcgcggga
ctgactatattctcccccactgctgcgctctctgg
ccaatacttgctgaccctgaaaatcgggagatttg
cgcaaacagctctcgtaactctagaagttaacgat
cgctgtttaaagatcgggtcgcagcttaacttttt
accgtcgaaatgctggacaacagaacagtatcaga
ctggatttcaaggcgaacacctttatccgatcgca
gacaccaatacacgacacgcggacgacgtatatcg
gggatacgaagatattctgcagcgctggaataatt
tgctgaggaaaaagaatcctagcgcgccagaccct
cgtccagatagcgtcccgcaagaaattcccgctgt
aaccaagaaagcggaagggcgcaccccggacgcag
aaagcagcgaaaagaaggcccctccagaagactcg
gaggacgacatgcaggcagaggcttctggagaaaa
tcctgccgccctccccgaagacgacgaagtccccg
aggacaccgagcacgatgatccaaactcggatcct
gactattacaatgacatgcccgccgtgatcccggt
ggaggagactactaaaagttctaatgccgtctcca
tgcccatattcgcggcgttcgtagcctgcgcggtc
gcgctcgtggggctactggtttggagcatcgtaaa
atgcgcgcgtagctaatcgagcctagaataggtgg
tttcttcctacatgccacgcctcacgctcataata
taaatcacatggaatagcataccaatgcctattca
ttgggacgttcgaaaagcatggcatcgctacttgg
aactctggctctccttgccgcgacgctcgcaccct
tcggcgcgatgggaatcgtgatcactggaaatcac
gtctccgccaggattgacgacgatcacatcgtgat
cgtcgcgcctcgccccgaagctacaattcaactgc
agctatttttcatgcctggccagagaccccacaaa
ccctactcaggaaccgtccgcgtcgcgtttcggtc
tgatataacaaaccagtgctaccaggaacttagcg
aggagcgctttgaaaattgcactcatcgatcgtct
tctgtttttgtcggctgtaaagtgaccgagtacac
gttctccgcctcgaacagactaaccggacctccac
acccgtttaagctcactatacgaaatcctcgtccg
aacgacagcgggatgttctacgtaattgttcggct
agacgacaccaaagaacccattgacgtcttcgcga
tccaactatcggtgtatcaattcgcgaacaccgcc
gcgactcgcggactctattccaaggcttcgtgtcg
caccttcggattacctaccgtccaacttgaggcct
atctcaggaccgaggaaagttggcgcaactggcaa
gcgtacgttgccacggaggccacgacgaccagcgc
cgaggcgacaaccccgacgcccgtcactgcaacca
gcgcctccgaacttgaagcggaacactttaccttt
ccctggctagaaaatggcgtggatcattacgaacc
gacacccgcaaacgaaaattcaaacgttactgtcc
gtctcgggacaatgagccctacgctaattggggta
accgtggctgccgtcgtgagcgcaacgatcggcct
cgtcattgtaatttccatcgtcaccagaaacatgt
gcaccccgcaccgaaaattagacacggtctcgcaa
gacgacgaagaacgttcccaaactagaagggaatc
gcgaaaatttggacccatggttgcgtgcgaaataa
acaagggggctgaccaggatagtgaacttgtggaa
ctggttgcgattgttaacccgtctgcgctaagctc
gcccgactcaataaaaatgtgattaagtctgaatg
tggctctccaatcatttcgattctctaatctccca
atcctctcaaaaggggcagtatcggacacggactg
ggaggggcgtacacgatagttatatggtacagcag
aggcctctgaacacttaggaggagaattcagccgg
ggagagcccctgttgagtaggcttgggagcatatt
gcaggatgaacatgttagtgatagttctcgcctct
tgtcttgcgcgcctaacttttgcgacgcgacacgt
cctctttttggaaggcactcaggctgtcctcgggg
aagatgatcccagaaacgttccggaagggactgta
atcaaatggacaaaagtcctgcggaacgcgtgcaa
gatgaaggcggccgatgtctgctcttcgcctaact
attgctttcatgatttaatttacgacggaggaaag
aaagactgcccgcccgcgggacccctgtctgcaaa
cctggtaattttactaaagcgcggcgaa
SEQ ID NO 10: mCMV IE promoter (1391 bp)
aactccgcccgttttatgactagaaccaatagttt
ttaatgccaaatgcactgaaatcccctaatttgca
aagccaaacgccccctatgtgagtaatacggggac
tttttacccaatttcccacgcggaaagccccctaa
tacactcatatggcatatgaatcagcacggtcatg
cactctaatggcggcccatagggactttccacata
gggggcgttcaccatttcccagcataggggtggtg
actcaatggcctttacccaagtacattgggtcaat
gggaggtaagccaatgggtttttcccattactggc
aagcacactgagtcaaatgggactttccactgggt
tttgcccaagtacattgggtcaatgggaggtgagc
caatgggaaaaacccattgctgccaagtacactga
ctcaatagggactttccaatgggtttttccattgt
tggcaagcatataaggtcaatgtgggtgagtcaat
agggactttccattgtattctgcccagtacataag
gtcaatagggggtgaatcaacaggaaagtcccatt
ggagccaagtacactgcgtcaatagggactttcca
ttgggttttgcccagtacataaggtcaatagggga
tgagtcaatgggaaaaacccattggagccaagtac
actgactcaatagggactttccattgggttttgcc
cagtacataaggtcaatagggggtgagtcaacagg
aaagttccattggagccaagtacattgagtcaata
gggactttccaatgggttttgcccagtacataagg
tcaatgggaggtaagccaatgggtttttcccatta
ctggcacgtatactgagtcattagggactttccaa
tgggttttgcccagtacataaggtcaataggggtg
aatcaacaggaaagtcccattggagccaagtacac
tgagtcaatagggactttccattgggttttgccca
gtacaaaaggtcaatagggggtgagtcaatgggtt
tttcccattattggcacgtacataaggtcaatagg
ggtgagtcattgggtttttccagccaatttaatta
aaacgccatgtactttcccaccattgacgtcaatg
ggctattgaaactaatgcaacgtgacctttaaacg
gtactttcccatagctgattaatgggaaagtaccg
ttctcgagccaatacacgtcaatgggaagtgaaag
ggcagccaaaacgtaacaccgccccggttttcccc
tggaaattccatattggcacgcattctattggctg
agctgcgttctacgtgggtataagaggcgcgacca
gcgtcggtaccgtcgcagtcttcggtctgaccacc
gtagaacgcagagctcctcgctgcag
SEQ ID NO 11:
chicken β-actin promoter (692 bp)
(Note: “nnn” denotes an ambiguous
sequence in highly GC-rich region.
Could be 3-5 “g's”)
cgcgccggatcagatctccatggtcgaggtgagcc
ccacgttctgcttcactctccccatctcccccccc
tccccacccccaattttgtatttatttatttttta
attattttgtgcagcgatgggggcggggggggggg
nnncgcgcgccaggcggggcggggcggggcgaggg
gcggggcggggcgaggcggagaggtgcggcggcag
ccaatcagagcggcgcgctccgaaagtttcctttt
atggcgaggcggcggcggcggcggccctataaaaa
gcgaagcgcgcggcgggcgggagtcgctgcgcgct
gccttcgccccgtgccccgctccgccgccgcctcg
cgccgcccgccccggctctgactgaccgcgttact
cccacaggtgagcgggcgggacggcccttctcctc
cgggctgtaattagcggcaggaaggaaatgggcgg
ggagggccttcgtgcgtcgccgcgccgccgtcccc
ttctccctctccagcctcggggctgtccgcggggg
gacggctgccttcgggggggacggggcagggcggg
gttcggcttctggcgtgtgaccggcggctctagag
cctctgctaaccatgttcatgccttcttctttttc
ctacagctcctgggcaacgtgctggttattgtgct
gtctcatcattttggcaaagaattgca
SEQ ID NO 12: hCMV IE promoter,
from strain AD169 (301 bp)
ggcagtacatctacgtattagtcatcgctattacc
atggtgatgcggttttggcagtacatcaatgggcg
tggatagcggtttgactcacggggatttccaagtc
tccaccccattgacgtcaatgggagtttgttttgg
caccaaaatcaacgggactttccaaaatgtcgtaa
caactccgccccattgacgcaaatgggcggtaggc
gtgtacggtgggaggtctatataagcagagctcgt
ttagtgaaccgtcagatcgcctggagacgccatcc
acgctgttttgacctccatag
SEQ ID NO 13:
FHV US-9 polyadenylation signal
(55 bp)
caataaacatagcatacgttatgacatggtctacc
gcgtcttatatggggacgac
SEQ ID NO 14: HSV TK polyadenylation
signal (370 bp)
gatccataattgattgacgggagatgg
gggaggctaactgaaacacggaaggagacaatacc
ggaaggaacccgcgctatgacggcaataaaaagac
agaataaaacgcacgggtgttgggtcgtttgttca
taaacgcggggttcggtcccagggctggcactctg
tcgataccccaccgagaccccattggggccaatac
gcccgcgtttcttccttttccccaccccacccccc
aagttcgggtgaaggcccagggctcgcagccaacg
tcggggcggcaggccctgccatagccactggcccc
gtgggttagggacggggtcccccatggggaatggt
ttatggttcgtgggggttattattttga
SEQ ID NO 15: 228509-ILT-435Vec6
(mCMV IEpro-VP2-SV40pA/ILT/HVT)
(14113 bp)
(IBDV + ILT gene cassettes in
HVT EcoRI#7 fragment. Virus no.
HVT/IBDV/ILT 1386-134)
gaattccagactaaatgccccggcccaatttgtca
agtgtgcagtcacggaggcgtcgaccgtgtccccg
gcattaaacaggaaagcgttaaagtttttgaatgt
taggtcacaggtacaaacataaatgtttgtacaaa
caggtaacaggtacaaacataaatgccccggcata
aatgtcccttacggcggatcgaaacgacattaggc
atactcgggtaccattttgcattccgatcagcacg
gatgaaattaggcaggaatgcggtttatattatgc
ggcattggacaaacgatatggcattgattggcagt
ttatgaatgtcttcatgttgggcgtaaacggattc
ctattggttcagaagacaacgacgatatatttaga
gagaaaaagctacccagcataggataaacacacat
tgagcattgagagacataggtatcggtatggatgg
gaaaactacacacgtgaacaccaaacgacttatat
actcgagcggtgatactactgagcaagaatgcact
gcatctgagccactgaatgaagactgtgatgaaaa
tgtgaccatcgatggaattggagaagaatatgcgc
agttcttcatgtccccgcaatgggtcccaaatcta
catcgcttgagcgaggataccaaaaaggtataccg
atgtatggtttccaacagactcaattattttccct
attatgaggcgttcaggcggtctttgtttgatatg
tatatgctaggtcggttggggcgtcgacttaagcg
atctgactgggagactattatgcatctgtcaccaa
cgcaaagtcggcgtctacatagaactttaagattt
gtggagcgtagaattatcccatctaacagttatat
acgcacatcgggccacgttccgccttcgagggcac
ttccgacagatacgaatttaaagatggatgaataa
ttaaattggaaagagtaactacattaatcgagcgt
catgacggcgtcccgtgaaaatgggaattttctac
tcgaaacaccgtgacatttgacagacctggaattg
ttattctgatatatagtgggtgtgtctggccggca
acatacataatgtgcatgcgaaaccactttttcag
tgtacgctgacattgtgcaacacggaggggtagca
tctacatacaatatatgttgattaatgattggaga
aaaaactatgcagctcgccgatcatatggctaact
cgccttcgtctatatggcggaccccgcgggaaaaa
tcgacgtaccatctgatttacaacaccagtaatga
acatgtcgcatccctgcccagatctgtgcgcccat
tggcgcggatcgttgtgaatgccgccgaaacactt
caggtcggtatgagagccgggaggccgccatcagc
aggagtttggcgagaggtgtttgatagaatgatga
cagccttccgtgaccacgagcctactgcgacattt
aatgctgcaaatcccattagaaaaatggtcgagac
agttctacagaataatgaagagcccccgcggacgc
atgctgaaatgggtaatcgccttatgaacattatg
tactggtgttgcttgggacacgcaggacaatgctc
gatatggcagttgtacgagacgaatcaggccattt
taagtttattagatgaagtggttatcggcacaaca
aatcccttttgcaccctcgagcaatactggaagcc
attatgcaccgcaatcgccaacaaggggacctcat
cgcttgttgaggatgccaaagtggccgagtacctg
gttagcatgcgcaaattgatataacataggcacgc
tctgatgttacagaccacaataccgcatacattta
ttgtaaggttgttaataaaggtttattctatgtaa
gactacaatactttcgacattgcttgtatacatat
taaatactttctcaagttcctattacataaaatgg
gatctatcattacattcgttaagagtctggataat
tttactgtttgccagcttcgatcttggaacgtact
gtggatagtgccttacttggaatcgtgaaaatttg
aaacgtccattatttggatatcttccggttgtccc
atatcccgccctggtaccgctcggataccttgccc
gtatggattcgtattgacagtcgcgcaatcgggga
ccaacaacgcgtgggtccacactcattcggaaatt
ttccgatgattctgaatatttattgccgctcgtta
cgagtcgttggacatatctgtaatacatttcttct
tctgaaggatcgctgcacatttgatctatacattg
gccaggatgttcaagtctcagatgttgcattctgg
cacagcacaactttatggcatttccgatgtaatcg
tccggcagccctgggggagttctatattcgcatat
tgggatggtaaggacaatagcagatctcgcaacct
ccagggaggctataataacgtttttaaaggatgga
tttctcataaaaatctgtcgcaaattacactgaga
atatcctttactagcgccgattgagagcatcgtcg
tccaattttctaaatggaaagaaaacaaggcgggc
aagagtgttccaaacattttcattttcggcgaatc
tctcaaatcccatggcgtgcaattgattgcaaaat
tggcacttccgttcacgtttgtatctccaaactct
aagacacttttaattgaaaaactacgttctagtgt
ggaaagaaacctataggcagaccatagaactattt
gacaccacatatctttttgtatgtcaaactgacca
tgatcgtatgttgctgaatgcactagggcaattcg
ctcgcgcgactccatacattgaataattccacacg
tcagctcatcggttagcaaggtccagtagttgaag
tcatttatttttccccgcggctggccaaatctacc
tctgggaatatccaagttgtcgaatatgatcgcac
cggctctggtcatggtgaaggaactgtagcataaa
gacgcaggtatcataggggtaatatttttttattc
actcacatactaaaagtaacgcatattagcaccat
gtatgggctatcaattgacatttgcgtagcactac
atcacgattatgtacaacataatgggacaacatat
ggcaagtagatgcaatttcctcacactagttgggt
ttatctactattgaattttcccctatctgtgatac
acttgggagcctctacaagcatattgccatcatgt
acgtttttatctactgtcttaacgcccatgggaac
ggaggcgtcgtcgtcatgtattggacggcaacata
ggcagcaacacaaattgcgtttaggtggggtgcat
gtggactcgataccaagcccctgcagctggggaac
gtctggtggagagccgataatttgatatacgcacg
ccatattactgtcgttgaagtacgccttatcttct
atgttttcaaatttaggttcccaagtggacgtgag
aagtgtttgtatctcacatggaatggcccaaggca
ttccagcccaggtgcctggtactttaatggcaaac
aaacgttttggtagaggtattgattctattgcagt
tctgcagatatctgcagccccgagtatccacaggc
tatacgatacgttatcggaggcaagctgcggccgc
tctagaactagtggatcccccgggctgcagcccaa
tgtggaattcgcccttgcacattgttactcctgca
tcttaaaaatatatcctgtagtaattttcacagca
atgtcataacatcatctcgctaaagaatgacctgg
gattggagaagtaatgaatatttgcaaccaatgca
ttgaataaactaacattaaacgaattcactagtgg
atcccccaactccgcccgttttatgactagaacca
atagtttttaatgccaaatgcactgaaatccccta
atttgcaaagccaaacgccccctatgtgagtaata
cggggactttttacccaatttcccacgcggaaagc
cccctaatacactcatatggcatatgaatcagcac
ggtcatgcactctaatggcggcccatagggacttt
ccacatagggggcgttcaccatttcccagcatagg
ggtggtgactcaatggcctttacccaagtacattg
ggtcaatgggaggtaagccaatgggtttttcccat
tactggcaagcacactgagtcaaatgggactttcc
actgggttttgcccaagtacattgggtcaatggga
ggtgagccaatgggaaaaacccattgctgccaagt
acactgactcaatagggactttccaatgggttttt
ccattgttggcaagcatataaggtcaatgtgggtg
agtcaatagggactttccattgtattctgcccagt
acataaggtcaatagggggtgaatcaacaggaaag
tcccattggagccaagtacactgcgtcaataggga
ctttccattgggttttgcccagtacataaggtcaa
taggggatgagtcaatgggaaaaacccattggagc
caagtacactgactcaatagggactttccattggg
ttttgcccagtacataaggtcaatagggggtgagt
caacaggaaagttccattggagccaagtacattga
gtcaatagggactttccaatgggttttgcccagta
cataaggtcaatgggaggtaagccaatgggttttt
cccattactggcacgtatactgagtcattagggac
tttccaatgggttttgcccagtacataaggtcaat
aggggtgaatcaacaggaaagtcccattggagcca
agtacactgagtcaatagggactttccattgggtt
ttgcccagtacaaaaggtcaatagggggtgagtca
atgggtttttcccattattggcacgtacataaggt
caataggggtgagtcattgggtttttccagccaat
ttaattaaaacgccatgtactttcccaccattgac
gtcaatgggctattgaaactaatgcaacgtgacct
ttaaacggtactttcccatagctgattaatgggaa
agtaccgttctcgagccaatacacgtcaatgggaa
gtgaaagggcagccaaaacgtaacaccgccccggt
tttcccctggaaattccatattggcacgcattcta
ttggctgagctgcgttctacgtgggtataagaggc
gcgaccagcgtcggtaccgtcgcagtcttcggtct
gaccaccgtagaacgcagagctcctcgctgcaggc
ggccgctctagaactcgtcgatcgcagcgatgaca
aacctgcaagatcaaacccaacagattgttccgtt
catacggagccttctgatgccaacaaccggaccgg
cgtccattccggacgacaccctggagaagcacact
ctcaggtcagagacctcgacctacaatttgactgt
gggggacacagggtcagggctaattgtctttttcc
ctggattccctggctcaattgtgggtgctcactac
acactgcagagcaatgggaactacaagttcgatca
gatgctcctgactgcccagaacctaccggccagct
acaactactgcagactagtgagtcggagtctcaca
gtgaggtcaagcacactccctggtggcgtttatgc
actaaacggcaccataaacgccgtgaccttccaag
gaagcctgagtgaactgacagatgttagctacaat
gggttgatgtctgcaacagccaacatcaacgacaa
aattgggaatgtcctggtaggggaaggggtcactg
tcctcagcctacccacatcatatgatcttgggtat
gtgaggcttggtgaccccattcccgctatagggct
tgacccaaaaatggtagctacatgcgacagcagtg
acaggcccagagtctacaccataactgcagccgat
gattaccaattctcatcacagtaccaaccaggtgg
ggtaacaatcacactgttctcagccaacattgatg
ctatcacaagcctcagcattgggggagagctcgtg
tttcaaacaagcgtccaaggccttgtactgggcgc
caccatctaccttataggctttgatgggactgcgg
taatcaccagagctgtggccgcagataatgggctg
acggccggcaccgacaatcttatgccattcaatct
tgtcattccaaccaatgagataacccagccaatca
catccatcaaactggagatagtgacctccaaaagt
ggtggtcaggcaggggatcagatgtcatggtcggc
aagtgggagcctagcagtgacgatccatggtggca
actatccaggggccctccgtcccgtcacactagta
gcctacgaaagagtggcaacaggatccgtcgttac
ggtcgctggggtgagtaacttcgagctgattccaa
atcctgaactagcaaagaacctggttacagaatac
ggccgatttgacccaggagccatgaactacacaaa
attgatactgagtgagagggaccgtcttggcatca
agaccgtctggccaacaagggagtacactgatttt
cgtgagtacttcatggaggtggccgacctcaactc
tcccctgaagattgcaggagcatttggcttcaaag
acataatccgggctataaggaggtaagcttcagac
atgataagatacattgatgagtttggacaaaccac
aactagaatgcagtgaaaaaaatgctttatttgtg
aaatttgtgatgctattgctttatttgtaaccatt
ataagctgcaataaacaagttaacaacaacaattg
cattcattttatgtttcaggttcagggggaggtgt
gggaggttttttcggatcctctagagtcgacggca
gagtcgcagacgcccctattggacgtcaaaattgt
agaggtgaagttttcaaacgatggcgaagtaacgg
cgacttgcgtttccaccgtcaaatctccctatagg
gtagaaactaattggaaagtagacctcgtagatgt
aatggatgaaatttctgggaacagtcccgccgggg
tttttaacagtaatgagaaatggcagaaacagctg
tactacagagtaaccgatggaagaacatcggtcca
gctaatgtgcctgtcgtgcacgagccattctccgg
aaccttactgtcttttcgacacgtctcttatagcg
agggaaaaagatatcgcgccagagttatactttac
ctctgatccgcaaacggcatactgcacaataactc
tgccgtccggcgttgttccgagattcgaatggagc
cttaataatgtttcactgccggaatatttgacggc
cacgaccgttgtttcgcataccgctggccaaagta
cagtgtggaagagcagcgcgagagcaggcgaggcg
tggatttctggccggggaggcaatatatacgaatg
caccgtcctcatctcagacggcactcgcgttacta
cgcgaaaggagaggtgcttaacaaacacatggatt
gcggtggaaaacggtgctgctcaggcgcagctgta
ttcactcttttctggacttgtgtcaggattatgcg
ggagcatatctgctttgtacgcaacgctatggacc
gccatttatttttgaggaatgctttttggactatc
gtactgctttcttccttcgctagccagagcaccgc
cgccgtcacgtacgactacattttaggccgtcgcg
cgctcgacgcgctaaccataccggcggttggcccg
tataacagatacctcactagggtatcaagaggctg
cgacgttgtcgagctcaacccgatttctaacgtgg
acgacatgatatcggcggccaaagaaaaagagaag
gggggccctttcgaggcctccgtcgtctggttcta
cgtgattaagggcgacgacggcgaggacaagtact
gtccaatctatagaaaagagtacagggaatgtggc
gacgtacaactgctatctgaatgcgccgttcaatc
tgcacagatgtgggcagtggactatgttcctagca
cccttgtatcgcgaaatggcgcgggactgactata
ttctcccccactgctgcgctctctggccaatactt
gctgaccctgaaaatcgggagatttgcgcaaacag
ctctcgtaactctagaagttaacgatcgctgttta
aagatcgggtcgcagcttaactttttaccgtcgaa
atgctggacaacagaacagtatcagactggatttc
aaggcgaacacctttatccgatcgcagacaccaat
acacgacacgcggacgacgtatatcggggatacga
agatattctgcagcgctggaataatttgctgagga
aaaagaatcctagcgcgccagaccctcgtccagat
agcgtcccgcaagaaattcccgctgtaaccaagaa
agcggaagggcgcaccccggacgcagaaagcagcg
aaaagaaggcccctccagaagactcggaggacgac
atgcaggcagaggcttctggagaaaatcctgccgc
cctccccgaagacgacgaagtccccgaggacaccg
agcacgatgatccaaactcggatcctgactattac
aatgacatgcccgccgtgatcccggtggaggagac
tactaaaagttctaatgccgtctccatgcccatat
tcgcggcgttcgtagcctgcgcggtcgcgctcgtg
gggctactggtttggagcatcgtaaaatgcgcgcg
tagctaatcgagcctagaataggtggtttcttcct
acatgccacgcctcacgctcataatataaatcaca
tggaatagcataccaatgcctattcattgggacgt
tcgaaaagcatggcatcgctacttggaactctggc
tctccttgccgcgacgctcgcacccttcggcgcga
tgggaatcgtgatcactggaaatcacgtctccgcc
aggattgacgacgatcacatcgtgatcgtcgcgcc
tcgccccgaagctacaattcaactgcagctatttt
tcatgcctggccagagaccccacaaaccctactca
ggaaccgtccgcgtcgcgtttcggtctgatataac
aaaccagtgctaccaggaacttagcgaggagcgct
ttgaaaattgcactcatcgatcgtcttctgttttt
gtcggctgtaaagtgaccgagtacacgttctccgc
ctcgaacagactaaccggacctccacacccgttta
agctcactatacgaaatcctcgtccgaacgacagc
gggatgttctacgtaattgttcggctagacgacac
caaagaacccattgacgtcttcgcgatccaactat
cggtgtatcaattcgcgaacaccgccgcgactcgc
ggactctattccaaggcttcgtgtcgcaccttcgg
attacctaccgtccaacttgaggcctatctcagga
ccgaggaaagttggcgcaactggcaagcgtacgtt
gccacggaggccacgacgaccagcgccgaggcgac
aaccccgacgcccgtcactgcaaccagcgcctccg
aacttgaagcggaacactttacctttccctggcta
gaaaatggcgtggatcattacgaaccgacacccgc
aaacgaaaattcaaacgttactgtccgtctcggga
caatgagccctacgctaattggggtaaccgtggct
gccgtcgtgagcgcaacgatcggcctcgtcattgt
aatttccatcgtcaccagaaacatgtgcaccccgc
accgaaaattagacacggtctcgcaagacgacgaa
gaacgttcccaaactagaagggaatcgcgaaaatt
tggacccatggttgcgtgcgaaataaacaaggggg
ctgaccaggatagtgaacttgtggaactggttgcg
attgttaacccgtctgcgctaagctcgcccgactc
aataaaaatgtgattaagtctgaatgtggctctcc
aatcatttcgattctctaatctcccaatcctctca
aaaggggcagtatcggacacggactgggaggggcg
tacacgatagttatatggtacagcagaggcctctg
aacacttaggaggagaattcagccggggagagccc
ctgttgagtaggcttgggagcatattgcaggatga
acatgttagtgatagttctcgcctcttgtcttgcg
cgcctaacttttgcgacgcgacacgtcctcttttt
ggaaggcactcaggctgtcctcggggaagatgatc
ccagaaacgttccggaagggactgtaatcaaatgg
acaaaagtcctgcggaacgcgtgcaagatgaaggc
ggccgatgtctgctcttcgcctaactattgctttc
atgatttaatttacgacggaggaaagaaagactgc
ccgcccgcgggacccctgtctgcaaacctggtaat
tttactaaagcgcggcgaagcttagcttgcctccg
attctagcattacatagccggtcagtagatcctgc
cattcggtagcgcaaccggctacatcttcaaacag
tctcacgataaatgcatctctcgttcctgccaatc
cggaaccgggcataccactcccgcctgccgattta
attctcacaattgggcgatgccggcggggcaaaac
gaatgtggatttggcaaaccgacacaggtctgctg
tacggactaatatgggcacacccacatcattcttc
agatgctccatgcattgttctatgagaaagatcca
tagggtggaggcagcgtcacgagatcgcccaggca
atcgatcgcattcgtctagtaaagtgacgagagtt
atcatgcacacacccatgcccacgccttccgaata
actggagctgtggaagatcggaaacgtctttttga
ctgccggtctcgtactactttcgcacaggtgtata
cccggacgcgtactatatattttatatcatccaac
gtccgaaattacatacgtggcggcgatggaagtag
atgttgagtcttcgaaagtaagtgcctcgaatatg
ggtattgtctgtgaaaatatcgaaagcggtacgac
ggttgcagaaccgtcgatgtcgccagatactagta
acaatagcttcgataacgaagacttccgtgggcct
gaatacgatgtggagataaataccagaaaatctgc
taatcttgatcgtatggaatcttcgtgccgtgaac
aacgagcggcgtgcgaacttcgaaagtgttcgtgt
cctacgtctgccgtgcgcatgcaatacagtattct
ttcatctctcgctccgggttcagagggtcatgtat
atatatgtactagatacggggacgcggaccaaaaa
aaatgcatagtgaaggcagtcgttggaggaaagaa
tcccgggagggaagtggatattttaaaaaccatct
cacataaatcaattataaaattaatccatgcctat
aaatggaaaaatgttgtgtgtatggcaatgcgtgt
atatcgttatgatcttttcacatatattgacggag
tcggccctatgccccttcaacagatgatctatatt
caacgtggactactagaggcgctagcatacataca
tgaaaggggcatcattcaccgagacgtaaagacgg
agaatatattcttggataatcacgaaaatgcagtt
ttgggtgacttcggtgctgcatgccaactaggaga
ttgtatagatacgccccaatgttacggttggagcg
gaactgtggaaacaaattcgccggaattatctgca
cttgatccgtattgcacaaaaacagatatttggag
tgccggattggttctatatgagatggcaattaaaa
atgtaccattgtttagtaagcaggtgaaaagttcg
ggatctcagctgagatccataatacggtgcatgca
agtgcatgaactggagtttccccgcaacgattcta
ccaacctctgtaaacatttcaaacaatatgcggtt
cgtgtacgaccgccttataccattcctcgagttat
aagaaatggggggatgccaatggatgttgaatatg
tcatttctaaaatgcttacgtttgaccaggagttc
agaccttctgctaaggaaatattgaatatgcccct
atttactaaggcgccgattaacctgcttaatatca
caccctctgacagtgtctaacggtatacaggcggg
agcgggtcgtggcgtcatcatcaccacttgagaat
ttatattttgaattgttgattgataaattaacctg
attcattgagaactgaaacgccatattggtttctt
ggatatgtctacaacaattagttaaattgctatgt
tctactgcgagtaacatttgataagttgtaagaga
cgggcgactcatgtcgaagttgacgaatataaagt
acataacgtgtttagaatacccagaatccgaatag
tccgcgggggcgtcttctcgcgtgagtaccaaata
ctgagttgaacttgaaaatgctaaatctgtgacac
tctttgtgtgatgattattgtcaccacttcgaaga
tggcttcgacattcatgatgttctggtgtttgttt
ggaatcgtaatagcgcttgtttcgtccaagtctga
caacaaagaaaatctgaagaattatatcacggata
agtcaaccaatattagaatacccacgccattattt
gtatcaacggaaaactcttatcccacaaaacatgt
aatctacgatgaaaactgtggcttcgctgtactca
atcctataagtgaccccaaatatgtccttttgagc
cagcttctaatgggaaggcgcaaatatgatgcgac
ggtcgcgtggtttgttctcggtaaaatgtgtgcca
gattaatatatttgcgcgaattttataactgctcg
acaaatgagccttttggcacatgttctatgagctc
tcctggatggtgggacaggcgctacgtctcaacca
gtttcatttctcgcgacgaattacagctggttttt
gcagcgccgtcccgagaattagatggtttatatac
gcgcgtagtagttgtcaacggggactttactacgg
ccgatataatgtttaatgttaaagtggcatgtgcc
ttttcaaagactggaatagaagatgatacattatg
caaaccctttcatttctttgccaatgcaacattgc
acaatttaaccatgattagatcggtaactcttcga
gcgcacgaaagccatttaaaggaatgggtggcacg
gagaggtggtaacgtccctgcagtgctacttgagt
ctaccatgtatcatgcatccaatctgcctagaaat
ttcagggatttctacataaagtctccagatgatta
taagtataatcacctagatgggccatctgtaatgc
tcatcactgacagacctagtgaagatttggatggg
aggctcgttcaccaaagtgacatttttactactac
aagtcctataaaacaggtccggtatgaagagcatc
agtcacatacaaagcagtatcctgtaaacaaaata
caagctataatttttttgatagggttaggctcgtt
cattggaagcatattcgtagttttggtagtatgga
ttatacgcagatattgcaatggagcgcggagtggg
ggaacgccccccagtcctcgccggtatgtgtatac
caggctatgatcacgtgtgaaacttgggcggacct
gtatcatatgtacaccgtccctattcgtttatagc
cagtacgtgttatctgcacatagaggaacatgtgt
catactgggatcgcatgcatggtatgtgtgactct
aatattattctgtatcataataaaaacacagtgca
tggtatatagaggatcgctggtaagcactacggta
gaccaatcggctcagattgcattctttggcatcga
taccgttgttaatttatatggcaaagtcttgttca
tgggagatcagtatttggaggaaatatactctgga
acgatggaaatactcaaatggaatcaagctaaccg
ctgctattctattgcgcatgcaacatattacgccg
actgtcctataatcagttctacggtattcagagga
tgccgggacgccgttgtttatactaggccccacag
cagaattc
SEQ ID NO 16: 1333-85.B6 (ILT/Chicken
β-actin pro-VP2-FHV US9pA /HVT)
(13064 bp)
(ILT + IBDV gene cassettes in
HVT EcoRI#7 fragment.)
Virus no. HVT/ILT/IBDV 1386-48.1.1.1
gaattccagactaaatgccccggcccaatttgtca
agtgtgcagtcacggaggcgtcgaccgtgtccccg
gcattaaacaggaaagcgttaaagtttttgaatgt
taggtcacaggtacaaacataaatgtttgtacaaa
caggtaacaggtacaaacataaatgccccggcata
aatgtcccttacggcggatcgaaacgacattaggc
atactcgggtaccattttgcattccgatcagcacg
gatgaaattaggcaggaatgcggtttatattatgc
ggcattggacaaacgatatggcattgattggcagt
ttatgaatgtcttcatgttgggcgtaaacggattc
ctattggttcagaagacaacgacgatatatttaga
gagaaaaagctacccagcataggataaacacacat
tgagcattgagagacataggtatcggtatggatgg
gaaaactacacacgtgaacaccaaacgacttatat
actcgagcggtgatactactgagcaagaatgcact
gcatctgagccactgaatgaagactgtgatgaaaa
tgtgaccatcgatggaattggagaagaatatgcgc
agttcttcatgtccccgcaatgggtcccaaatcta
catcgcttgagcgaggataccaaaaaggtataccg
atgtatggtttccaacagactcaattattttccct
attatgaggcgttcaggcggtctttgtttgatatg
tatatgctaggtcggttggggcgtcgacttaagcg
atctgactgggagactattatgcatctgtcaccaa
cgcaaagtcggcgtctacatagaactttaagattt
gtggagcgtagaattatcccatctaacagttatat
acgcacatcgggccacgttccgccttcgagggcac
ttccgacagatacgaatttaaagatggatgaataa
ttaaattggaaagagtaactacattaatcgagcgt
catgacggcgtcccgtgaaaatgggaattttctac
tcgaaacaccgtgacatttgacagacctggaattg
ttattctgatatatagtgggtgtgtctggccggca
acatacataatgtgcatgcgaaaccactttttcag
tgtacgctgacattgtgcaacacggaggggtagca
tctacatacaatatatgttgattaatgattggaga
aaaaactatgcagctcgccgatcatatggctaact
cgccttcgtctatatggcggaccccgcgggaaaaa
tcgacgtaccatctgatttacaacaccagtaatga
acatgtcgcatccctgcccagatctgtgcgcccat
tggcgcggatcgttgtgaatgccgccgaaacactt
caggtcggtatgagagccgggaggccgccatcagc
aggagtttggcgagaggtgtttgatagaatgatga
cagccttccgtgaccacgagcctactgcgacattt
aatgctgcaaatcccattagaaaaatggtcgagac
agttctacagaataatgaagagcccccgcggacgc
atgctgaaatgggtaatcgccttatgaacattatg
tactggtgttgcttgggacacgcaggacaatgctc
gatatggcagttgtacgagacgaatcaggccattt
taagtttattagatgaagtggttatcggcacaaca
aatcccttttgcaccctcgagcaatactggaagcc
attatgcaccgcaatcgccaacaaggggacctcat
cgcttgttgaggatgccaaagtggccgagtacctg
gttagcatgcgcaaattgatataacataggcacgc
tctgatgttacagaccacaataccgcatacattta
ttgtaaggttgttaataaaggtttattctatgtaa
gactacaatactttcgacattgcttgtatacatat
taaatactttctcaagttcctattacataaaatgg
gatctatcattacattcgttaagagtctggataat
tttactgtttgccagcttcgatcttggaacgtact
gtggatagtgccttacttggaatcgtgaaaatttg
aaacgtccattatttggatatcttccggttgtccc
atatcccgccctggtaccgctcggataccttgccc
gtatggattcgtattgacagtcgcgcaatcgggga
ccaacaacgcgtgggtccacactcattcggaaatt
ttccgatgattctgaatatttattgccgctcgtta
cgagtcgttggacatatctgtaatacatttcttct
tctgaaggatcgctgcacatttgatctatacattg
gccaggatgttcaagtctcagatgttgcattctgg
cacagcacaactttatggcatttccgatgtaatcg
tccggcagccctgggggagttctatattcgcatat
tgggatggtaaggacaatagcagatctcgcaacct
ccagggaggctataataacgtttttaaaggatgga
tttctcataaaaatctgtcgcaaattacactgaga
atatcctttactagcgccgattgagagcatcgtcg
tccaattttctaaatggaaagaaaacaaggcgggc
aagagtgttccaaacattttcattttcggcgaatc
tctcaaatcccatggcgtgcaattgattgcaaaat
tggcacttccgttcacgtttgtatctccaaactct
aagacacttttaattgaaaaactacgttctagtgt
ggaaagaaacctataggcagaccatagaactattt
gacaccacatatctttttgtatgtcaaactgacca
tgatcgtatgttgctgaatgcactagggcaattcg
ctcgcgcgactccatacattgaataattccacacg
tcagctcatcggttagcaaggtccagtagttgaag
tcatttatttttccccgcggctggccaaatctacc
tctgggaatatccaagttgtcgaatatgatcgcac
cggctctggtcatggtgaaggaactgtagcataaa
gacgcaggtatcataggggtaatatttttttattc
actcacatactaaaagtaacgcatattagcaccat
gtatgggctatcaattgacatttgcgtagcactac
atcacgattatgtacaacataatgggacaacatat
ggcaagtagatgcaatttcctcacactagttgggt
ttatctactattgaattttcccctatctgtgatac
acttgggagcctctacaagcatattgccatcatgt
acgtttttatctactgtcttaacgcccatgggaac
ggaggcgtcgtcgtcatgtattggacggcaacata
ggcagcaacacaaattgcgtttaggtggggtgcat
gtggactcgataccaagcccctgcagctggggaac
gtctggtggagagccgataatttgatatacgcacg
ccatattactgtcgttgaagtacgccttatcttct
atgttttcaaatttaggttcccaagtggacgtgag
aagtgtttgtatctcacatggaatggcccaaggca
ttccagcccaggtgcctggtactttaatggcaaac
aaacgttttggtagaggtattgattctattgcagt
tctgcagatatctgcagccccgagtatccacaggc
tatacgatacgttatcggaggcaagcttaattaag
taccgagctcgaattggcgcgcccgacggcagagt
cgcagacgcccctattggacgtcaaaattgtagag
gtgaagttttcaaacgatggcgaagtaacggcgac
ttgcgtttccaccgtcaaatctccctatagggtag
aaactaattggaaagtagacctcgtagatgtaatg
gatgaaatttctgggaacagtcccgccggggtttt
taacagtaatgagaaatggcagaaacagctgtact
acagagtaaccgatggaagaacatcggtccagcta
atgtgcctgtcgtgcacgagccattctccggaacc
ttactgtcttttcgacacgtctcttatagcgaggg
aaaaagatatcgcgccagagttatactttacctct
gatccgcaaacggcatactgcacaataactctgcc
gtccggcgttgttccgagattcgaatggagcctta
ataatgtttcactgccggaatatttgacggccacg
accgttgtttcgcataccgctggccaaagtacagt
gtggaagagcagcgcgagagcaggcgaggcgtgga
tttctggccggggaggcaatatatacgaatgcacc
gtcctcatctcagacggcactcgcgttactacgcg
aaaggagaggtgcttaacaaacacatggattgcgg
tggaaaacggtgctgctcaggcgcagctgtattca
ctcttttctggacttgtgtcaggattatgcgggag
catatctgctttgtacgcaacgctatggaccgcca
tttatttttgaggaatgctttttggactatcgtac
tgctttcttccttcgctagccagagcaccgccgcc
gtcacgtacgactacattttaggccgtcgcgcgct
cgacgcgctaaccataccggcggttggcccgtata
acagatacctcactagggtatcaagaggctgcgac
gttgtcgagctcaacccgatttctaacgtggacga
catgatatcggcggccaaagaaaaagagaaggggg
gccctttcgaggcctccgtcgtctggttctacgtg
attaagggcgacgacggcgaggacaagtactgtcc
aatctatagaaaagagtacagggaatgtggcgacg
tacaactgctatctgaatgcgccgttcaatctgca
cagatgtgggcagtggactatgttcctagcaccct
tgtatcgcgaaatggcgcgggactgactatattct
cccccactgctgcgctctctggccaatacttgctg
accctgaaaatcgggagatttgcgcaaacagctct
cgtaactctagaagttaacgatcgctgtttaaaga
tcgggtcgcagcttaactttttaccgtcgaaatgc
tggacaacagaacagtatcagactggatttcaagg
cgaacacctttatccgatcgcagacaccaatacac
gacacgcggacgacgtatatcggggatacgaagat
attctgcagcgctggaataatttgctgaggaaaaa
gaatcctagcgcgccagaccctcgtccagatagcg
tcccgcaagaaattcccgctgtaaccaagaaagcg
gaagggcgcaccccggacgcagaaagcagcgaaaa
gaaggcccctccagaagactcggaggacgacatgc
aggcagaggcttctggagaaaatcctgccgccctc
cccgaagacgacgaagtccccgaggacaccgagca
cgatgatccaaactcggatcctgactattacaatg
acatgcccgccgtgatcccggtggaggagactact
aaaagttctaatgccgtctccatgcccatattcgc
ggcgttcgtagcctgcgcggtcgcgctcgtggggc
tactggtttggagcatcgtaaaatgcgcgcgtagc
taatcgagcctagaataggtggtttcttcctacat
gccacgcctcacgctcataatataaatcacatgga
atagcataccaatgcctattcattgggacgttcga
aaagcatggcatcgctacttggaactctggctctc
cttgccgcgacgctcgcacccttcggcgcgatggg
aatcgtgatcactggaaatcacgtctccgccagga
ttgacgacgatcacatcgtgatcgtcgcgcctcgc
cccgaagctacaattcaactgcagctatttttcat
gcctggccagagaccccacaaaccctactcaggaa
ccgtccgcgtcgcgtttcggtctgatataacaaac
cagtgctaccaggaacttagcgaggagcgctttga
aaattgcactcatcgatcgtcttctgtttttgtcg
gctgtaaagtgaccgagtacacgttctccgcctcg
aacagactaaccggacctccacacccgtttaagct
cactatacgaaatcctcgtccgaacgacagcggga
tgttctacgtaattgttcggctagacgacaccaaa
gaacccattgacgtcttcgcgatccaactatcggt
gtatcaattcgcgaacaccgccgcgactcgcggac
tctattccaaggcttcgtgtcgcaccttcggatta
cctaccgtccaacttgaggcctatctcaggaccga
ggaaagttggcgcaactggcaagcgtacgttgcca
cggaggccacgacgaccagcgccgaggcgacaacc
ccgacgcccgtcactgcaaccagcgcctccgaact
tgaagcggaacactttacctttccctggctagaaa
atggcgtggatcattacgaaccgacacccgcaaac
gaaaattcaaacgttactgtccgtctcgggacaat
gagccctacgctaattggggtaaccgtggctgccg
tcgtgagcgcaacgatcggcctcgtcattgtaatt
tccatcgtcaccagaaacatgtgcaccccgcaccg
aaaattagacacggtctcgcaagacgacgaagaac
gttcccaaactagaagggaatcgcgaaaatttgga
cccatggttgcgtgcgaaataaacaagggggctga
ccaggatagtgaacttgtggaactggttgcgattg
ttaacccgtctgcgctaagctcgcccgactcaata
aaaatgtgattaagtctgaatgtggctctccaatc
atttcgattctctaatctcccaatcctctcaaaag
gggcagtatcggacacggactgggaggggcgtaca
cgatagttatatggtacagcagaggcctctgaaca
cttaggaggagaattcagccggggagagcccctgt
tgagtaggcttgggagcatattgcaggatgaacat
gttagtgatagttctcgcctcttgtcttgcgcgcc
taacttttgcgacgcgacacgtcctctttttggaa
ggcactcaggctgtcctcggggaagatgatcccag
aaacgttccggaagggactgtaatcaaatggacaa
aagtcctgcggaacgcgtgcaagatgaaggcggcc
gatgtctgctcttcgcctaactattgctttcatga
tttaatttacgacggaggaaagaaagactgcccgc
ccgcgggacccctgtctgcaaacctggtaatttta
ctaaagcgcggcgggcgcgccggatcagatctcca
tggtcgaggtgagccccacgttctgcttcactctc
cccatctcccccccctccccacccccaattttgta
tttatttattttttaattattttgtgcagcgatgg
gggcgggggggggggnnncgcgcgccaggcggggc
ggggcggggcgaggggcggggcggggcgaggcgga
gaggtgcggcggcagccaatcagagcggcgcgctc
cgaaagtttccttttatggcgaggcggcggcggcg
gcggccctataaaaagcgaagcgcgcggcgggcgg
gagtcgctgcgcgctgccttcgccccgtgccccgc
tccgccgccgcctcgcgccgcccgccccggctctg
actgaccgcgttactcccacaggtgagcgggcggg
acggcccttctcctccgggctgtaattagcggcag
gaaggaaatgggcggggagggccttcgtgcgtcgc
cgcgccgccgtccccttctccctctccagcctcgg
ggctgtccgcggggggacggctgccttcggggggg
acggggcagggcggggttcggcttctggcgtgtga
ccggcggctctagagcctctgctaaccatgttcat
gccttcttctttttcctacagctcctgggcaacgt
gctggttattgtgctgtctcatcattttggcaaag
aattgcagatctggatctatgacaaacctgcaaga
tcaaacccaacagattgttccgttcatacggagcc
ttctgatgccaacaaccggaccggcgtccattccg
gacgacaccctggagaagcacactctcaggtcaga
gacctcgacctacaatttgactgtgggggacacag
ggtcagggctaattgtctttttccctggattccct
ggctcaattgtgggtgctcactacacactgcagag
caatgggaactacaagttcgatcagatgctcctga
ctgcccagaacctaccggccagctacaactactgc
agactagtgagtcggagtctcacagtgaggtcaag
cacactccctggtggcgtttatgcactaaacggca
ccataaacgccgtgaccttccaaggaagcctgagt
gaactgacagatgttagctacaatgggttgatgtc
tgcaacagccaacatcaacgacaaagttgggaatg
tcctggtaggggaaggggtcactgtcctcagccta
cccacatcatatgatcttgggtatgtgaggcttgg
tgaccccattcccgctatagggcttgacccaaaaa
tggtagctacatgcgacagcagtgacaggcccaga
gtctacaccataactgcagccgatgattaccaatt
ctcatcacagtaccaaccaggtggggtaacaatca
cactgttctcagccaacattgatgctatcacaagc
ctcagcattgggggagagctcgtgtttcaaacaag
cgtccaaggccttgtactgggcgccaccatctacc
ttataggctttgatgggactgcggtaatcaccaga
gctgtggccgcagataatgggctgacggccggcac
cgacaatcttatgccattcaatcttgtcattccaa
ccaatgagataacccagccgatcacatccatcaaa
ctggagatagtgacctccaaaagtggtggtcaggc
aggggatcagatgtcatggtcggcaagtgggagcc
tagcagtgacgatccatggtggcaactatccaggg
gccctccgtcccgtcacactagtagcctacgaaag
agtggcaacaggatccgtcgttacggtcgctgggg
tgagtaacttcgagctgatcccaaatcctgaacta
gcaaagaacctggttacagaatacggccgatttga
cccaggagccatgaactacacaaaattgatactga
gtgagagggaccgtcttggcatcaagaccgtctgg
ccaacaagggagtacactgattttcgtgagtactt
catggaggtggccgacctcaactctcccctgaaga
ttgcaggagcatttggcttcaaagacataatccgg
gctataaggaggtaagatccgatctctcgattaat
taacaataaacatagcatacgttatgacatggtct
accgcgtcttatatggggacgacaagcttgcctcc
gattctagcattacatagccggtcagtagatcctg
ccattcggtagcgcaaccggctacatcttcaaaca
gtctcacgataaatgcatctctcgttcctgccaat
ccggaaccgggcataccactcccgcctgccgattt
aattctcacaattgggcgatgccggcggggcaaaa
cgaatgtggatttggcaaaccgacacaggtctgct
gtacggactaatatgggcacacccacatcattctt
cagatgctccatgcattgttctatgagaaagatcc
atagggtggaggcagcgtcacgagatcgcccaggc
aatcgatcgcattcgtctagtaaagtgacgagagt
tatcatgcacacacccatgcccacgccttccgaat
aactggagctgtggaagatcggaaacgtctttttg
actgccggtctcgtactactttcgcacaggtgtat
acccggacgcgtactatatattttatatcatccaa
cgtccgaaattacatacgtggcggcgatggaagta
gatgttgagtcttcgaaagtaagtgcctcgaatat
gggtattgtctgtgaaaatatcgaaagcggtacga
cggttgcagaaccgtcgatgtcgccagatactagt
aacaatagcttcgataacgaagacttccgtgggcc
tgaatacgatgtggagataaataccagaaaatctg
ctaatcttgatcgtatggaatcttcgtgccgtgaa
caacgagcggcgtgcgaacttcgaaagtgttcgtg
tcctacgtctgccgtgcgcatgcaatacagtattc
tttcatctctcgctccgggttcagagggtcatgta
tatatatgtactagatacggggacgcggaccaaaa
aaaatgcatagtgaaggcagtcgttggaggaaaga
atcccgggagggaagtggatattttaaaaaccatc
tcacataaatcaattataaaattaatccatgccta
taaatggaaaaatgttgtgtgtatggcaatgcgtg
tatatcgttatgatcttttcacatatattgacgga
gtcggccctatgccccttcaacagatgatctatat
tcaacgtggactactagaggcgctagcatacatac
atgaaaggggcatcattcaccgagacgtaaagacg
gagaatatattcttggataatcacgaaaatgcagt
tttgggtgacttcggtgctgcatgccaactaggag
attgtatagatacgccccaatgttacggttggagc
ggaactgtggaaacaaattcgccggaattatctgc
acttgatccgtattgcacaaaaacagatatttgga
gtgccggattggttctatatgagatggcaattaaa
aatgtaccattgtttagtaagcaggtgaaaagttc
gggatctcagctgagatccataatacggtgcatgc
aagtgcatgaactggagtttccccgcaacgattct
accaacctctgtaaacatttcaaacaatatgcggt
tcgtgtacgaccgccttataccattcctcgagtta
taagaaatggggggatgccaatggatgttgaatat
gtcatttctaaaatgcttacgtttgaccaggagtt
cagaccttctgctaaggaaatattgaatatgcccc
tatttactaaggcgccgattaacctgcttaatatc
acaccctctgacagtgtctaacggtatacaggcgg
gagcgggtcgtggcgtcatcatcaccacttgagaa
tttatattttgaattgttgattgataaattaacct
gattcattgagaactgaaacgccatattggtttct
tggatatgtctacaacaattagttaaattgctatg
ttctactgcgagtaacatttgataagttgtaagag
acgggcgactcatgtcgaagttgacgaatataaag
tacataacgtgtttagaatacccagaatccgaata
gtccgcgggggcgtcttctcgcgtgagtaccaaat
actgagttgaacttgaaaatgctaaatctgtgaca
ctctttgtgtgatgattattgtcaccacttcgaag
atggcttcgacattcatgatgttctggtgtttgtt
tggaatcgtaatagcgcttgtttcgtccaagtctg
acaacaaagaaaatctgaagaattatatcacggat
aagtcaaccaatattagaatacccacgccattatt
tgtatcaacggaaaactcttatcccacaaaacatg
taatctacgatgaaaactgtggcttcgctgtactc
aatcctataagtgaccccaaatatgtccttttgag
ccagcttctaatgggaaggcgcaaatatgatgcga
cggtcgcgtggtttgttctcggtaaaatgtgtgcc
agattaatatatttgcgcgaattttataactgctc
gacaaatgagccttttggcacatgttctatgagct
ctcctggatggtgggacaggcgctacgtctcaacc
agtttcatttctcgcgacgaattacagctggtttt
tgcagcgccgtcccgagaattagatggtttatata
cgcgcgtagtagttgtcaacggggactttactacg
gccgatataatgtttaatgttaaagtggcatgtgc
cttttcaaagactggaatagaagatgatacattat
gcaaaccctttcatttctttgccaatgcaacattg
cacaatttaaccatgattagatcggtaactcttcg
agcgcacgaaagccatttaaaggaatgggtggcac
ggagaggtggtaacgtccctgcagtgctacttgag
tctaccatgtatcatgcatccaatctgcctagaaa
tttcagggatttctacataaagtctccagatgatt
ataagtataatcacctagatgggccatctgtaatg
ctcatcactgacagacctagtgaagatttggatgg
gaggctcgttcaccaaagtgacatttttactacta
caagtcctataaaacaggtccggtatgaagagcat
cagtcacatacaaagcagtatcctgtaaacaaaat
acaagctataatttttttgatagggttaggctcgt
tcattggaagcatattcgtagttttggtagtatgg
attatacgcagatattgcaatggagcgcggagtgg
gggaacgccccccagtcctcgccggtatgtgtata
ccaggctatgatcacgtgtgaaacttgggcggacc
tgtatcatatgtacaccgtccctattcgtttatag
ccagtacgtgttatctgcacatagaggaacatgtg
tcatactgggatcgcatgcatggtatgtgtgactc
taatattattctgtatcataataaaaacacagtgc
atggtatatagaggatcgctggtaagcactacggt
agaccaatcggctcagattgcattctttggcatcg
ataccgttgttaatttatatggcaaagtcttgttc
atgggagatcagtatttggaggaaatatactctgg
aacgatggaaatactcaaatggaatcaagctaacc
gctgctattctattgcgcatgcaacatattacgcc
gactgtcctataatcagttctacggtattcagagg
atgccgggacgccgttgtttatactaggccccaca
gcagaattc
SEQ ID NO 17: 1386-04.4#1 (ILT/hCMV
IEpro-VP2-HSV TKpA/HVT) (13017 bp)
(ILT + IBDV gene cassettes in
HVT EcoRI#7 fragment. Virus no.
HVT/ILT/IBDV 1386-48.3.1.7)
gaattccagactaaatgccccggcccaatttgtca
agtgtgcagtcacggaggcgtcgaccgtgtccccg
gcattaaacaggaaagcgttaaagtttttgaatgt
taggtcacaggtacaaacataaatgtttgtacaaa
caggtaacaggtacaaacataaatgccccggcata
aatgtcccttacggcggatcgaaacgacattaggc
atactcgggtaccattttgcattccgatcagcacg
gatgaaattaggcaggaatgcggtttatattatgc
ggcattggacaaacgatatggcattgattggcagt
ttatgaatgtcttcatgttgggcgtaaacggattc
ctattggttcagaagacaacgacgatatatttaga
gagaaaaagctacccagcataggataaacacacat
tgagcattgagagacataggtatcggtatggatgg
gaaaactacacacgtgaacaccaaacgacttatat
actcgagcggtgatactactgagcaagaatgcact
gcatctgagccactgaatgaagactgtgatgaaaa
tgtgaccatcgatggaattggagaagaatatgcgc
agttcttcatgtccccgcaatgggtcccaaatcta
catcgcttgagcgaggataccaaaaaggtataccg
atgtatggtttccaacagactcaattattttccct
attatgaggcgttcaggcggtctttgtttgatatg
tatatgctaggtcggttggggcgtcgacttaagcg
atctgactgggagactattatgcatctgtcaccaa
cgcaaagtcggcgtctacatagaactttaagattt
gtggagcgtagaattatcccatctaacagttatat
acgcacatcgggccacgttccgccttcgagggcac
ttccgacagatacgaatttaaagatggatgaataa
ttaaattggaaagagtaactacattaatcgagcgt
catgacggcgtcccgtgaaaatgggaattttctac
tcgaaacaccgtgacatttgacagacctggaattg
ttattctgatatatagtgggtgtgtctggccggca
acatacataatgtgcatgcgaaaccactttttcag
tgtacgctgacattgtgcaacacggaggggtagca
tctacatacaatatatgttgattaatgattggaga
aaaaactatgcagctcgccgatcatatggctaact
cgccttcgtctatatggcggaccccgcgggaaaaa
tcgacgtaccatctgatttacaacaccagtaatga
acatgtcgcatccctgcccagatctgtgcgcccat
tggcgcggatcgttgtgaatgccgccgaaacactt
caggtcggtatgagagccgggaggccgccatcagc
aggagtttggcgagaggtgtttgatagaatgatga
cagccttccgtgaccacgagcctactgcgacattt
aatgctgcaaatcccattagaaaaatggtcgagac
agttctacagaataatgaagagcccccgcggacgc
atgctgaaatgggtaatcgccttatgaacattatg
tactggtgttgcttgggacacgcaggacaatgctc
gatatggcagttgtacgagacgaatcaggccattt
taagtttattagatgaagtggttatcggcacaaca
aatcccttttgcaccctcgagcaatactggaagcc
attatgcaccgcaatcgccaacaaggggacctcat
cgcttgttgaggatgccaaagtggccgagtacctg
gttagcatgcgcaaattgatataacataggcacgc
tctgatgttacagaccacaataccgcatacattta
ttgtaaggttgttaataaaggtttattctatgtaa
gactacaatactttcgacattgcttgtatacatat
taaatactttctcaagttcctattacataaaatgg
gatctatcattacattcgttaagagtctggataat
tttactgtttgccagcttcgatcttggaacgtact
gtggatagtgccttacttggaatcgtgaaaatttg
aaacgtccattatttggatatcttccggttgtccc
atatcccgccctggtaccgctcggataccttgccc
gtatggattcgtattgacagtcgcgcaatcgggga
ccaacaacgcgtgggtccacactcattcggaaatt
ttccgatgattctgaatatttattgccgctcgtta
cgagtcgttggacatatctgtaatacatttcttct
tctgaaggatcgctgcacatttgatctatacattg
gccaggatgttcaagtctcagatgttgcattctgg
cacagcacaactttatggcatttccgatgtaatcg
tccggcagccctgggggagttctatattcgcatat
tgggatggtaaggacaatagcagatctcgcaacct
ccagggaggctataataacgtttttaaaggatgga
tttctcataaaaatctgtcgcaaattacactgaga
atatcctttactagcgccgattgagagcatcgtcg
tccaattttctaaatggaaagaaaacaaggcgggc
aagagtgttccaaacattttcattttcggcgaatc
tctcaaatcccatggcgtgcaattgattgcaaaat
tggcacttccgttcacgtttgtatctccaaactct
aagacacttttaattgaaaaactacgttctagtgt
ggaaagaaacctataggcagaccatagaactattt
gacaccacatatctttttgtatgtcaaactgacca
tgatcgtatgttgctgaatgcactagggcaattcg
ctcgcgcgactccatacattgaataattccacacg
tcagctcatcggttagcaaggtccagtagttgaag
tcatttatttttccccgcggctggccaaatctacc
tctgggaatatccaagttgtcgaatatgatcgcac
cggctctggtcatggtgaaggaactgtagcataaa
gacgcaggtatcataggggtaatatttttttattc
actcacatactaaaagtaacgcatattagcaccat
gtatgggctatcaattgacatttgcgtagcactac
atcacgattatgtacaacataatgggacaacatat
ggcaagtagatgcaatttcctcacactagttgggt
ttatctactattgaattttcccctatctgtgatac
acttgggagcctctacaagcatattgccatcatgt
acgtttttatctactgtcttaacgcccatgggaac
ggaggcgtcgtcgtcatgtattggacggcaacata
ggcagcaacacaaattgcgtttaggtggggtgcat
gtggactcgataccaagcccctgcagctggggaac
gtctggtggagagccgataatttgatatacgcacg
ccatattactgtcgttgaagtacgccttatcttct
atgttttcaaatttaggttcccaagtggacgtgag
aagtgtttgtatctcacatggaatggcccaaggca
ttccagcccaggtgcctggtactttaatggcaaac
aaacgttttggtagaggtattgattctattgcagt
tctgcagatatctgcagccccgagtatccacaggc
tatacgatacgttatcggaggcaagcttgttaatt
aagtcgacggcagagtcgcagacgcccctattgga
cgtcaaaattgtagaggtgaagttttcaaacgatg
gcgaagtaacggcgacttgcgtttccaccgtcaaa
tctccctatagggtagaaactaattggaaagtaga
cctcgtagatgtaatggatgaaatttctgggaaca
gtcccgccggggtttttaacagtaatgagaaatgg
cagaaacagctgtactacagagtaaccgatggaag
aacatcggtccagctaatgtgcctgtcgtgcacga
gccattctccggaaccttactgtcttttcgacacg
tctcttatagcgagggaaaaagatatcgcgccaga
gttatactttacctctgatccgcaaacggcatact
gcacaataactctgccgtccggcgttgttccgaga
ttcgaatggagccttaataatgtttcactgccgga
atatttgacggccacgaccgttgtttcgcataccg
ctggccaaagtacagtgtggaagagcagcgcgaga
gcaggcgaggcgtggatttctggccggggaggcaa
tatatacgaatgcaccgtcctcatctcagacggca
ctcgcgttactacgcgaaaggagaggtgcttaaca
aacacatggattgcggtggaaaacggtgctgctca
ggcgcagctgtattcactcttttctggacttgtgt
caggattatgcgggagcatatctgctttgtacgca
acgctatggaccgccatttatttttgaggaatgct
ttttggactatcgtactgctttcttccttcgctag
ccagagcaccgccgccgtcacgtacgactacattt
taggccgtcgcgcgctcgacgcgctaaccataccg
gcggttggcccgtataacagatacctcactagggt
atcaagaggctgcgacgttgtcgagctcaacccga
tttctaacgtggacgacatgatatcggcggccaaa
gaaaaagagaaggggggccctttcgaggcctccgt
cgtctggttctacgtgattaagggcgacgacggcg
aggacaagtactgtccaatctatagaaaagagtac
agggaatgtggcgacgtacaactgctatctgaatg
cgccgttcaatctgcacagatgtgggcagtggact
atgttcctagcacccttgtatcgcgaaatggcgcg
ggactgactatattctcccccactgctgcgctctc
tggccaatacttgctgaccctgaaaatcgggagat
ttgcgcaaacagctctcgtaactctagaagttaac
gatcgctgtttaaagatcgggtcgcagcttaactt
tttaccgtcgaaatgctggacaacagaacagtatc
agactggatttcaaggcgaacacctttatccgatc
gcagacaccaatacacgacacgcggacgacgtata
tcggggatacgaagatattctgcagcgctggaata
atttgctgaggaaaaagaatcctagcgcgccagac
cctcgtccagatagcgtcccgcaagaaattcccgc
tgtaaccaagaaagcggaagggcgcaccccggacg
cagaaagcagcgaaaagaaggcccctccagaagac
tcggaggacgacatgcaggcagaggcttctggaga
aaatcctgccgccctccccgaagacgacgaagtcc
ccgaggacaccgagcacgatgatccaaactcggat
cctgactattacaatgacatgcccgccgtgatccc
ggtggaggagactactaaaagttctaatgccgtct
ccatgcccatattcgcggcgttcgtagcctgcgcg
gtcgcgctcgtggggctactggtttggagcatcgt
aaaatgcgcgcgtagctaatcgagcctagaatagg
tggtttcttcctacatgccacgcctcacgctcata
atataaatcacatggaatagcataccaatgcctat
tcattgggacgttcgaaaagcatggcatcgctact
tggaactctggctctccttgccgcgacgctcgcac
ccttcggcgcgatgggaatcgtgatcactggaaat
cacgtctccgccaggattgacgacgatcacatcgt
gatcgtcgcgcctcgccccgaagctacaattcaac
tgcagctatttttcatgcctggccagagaccccac
aaaccctactcaggaaccgtccgcgtcgcgtttcg
gtctgatataacaaaccagtgctaccaggaactta
gcgaggagcgctttgaaaattgcactcatcgatcg
tcttctgtttttgtcggctgtaaagtgaccgagta
cacgttctccgcctcgaacagactaaccggacctc
cacacccgtttaagctcactatacgaaatcctcgt
ccgaacgacagcgggatgttctacgtaattgttcg
gctagacgacaccaaagaacccattgacgtcttcg
cgatccaactatcggtgtatcaattcgcgaacacc
gccgcgactcgcggactctattccaaggcttcgtg
tcgcaccttcggattacctaccgtccaacttgagg
cctatctcaggaccgaggaaagttggcgcaactgg
caagcgtacgttgccacggaggccacgacgaccag
cgccgaggcgacaaccccgacgcccgtcactgcaa
ccagcgcctccgaacttgaagcggaacactttacc
tttccctggctagaaaatggcgtggatcattacga
accgacacccgcaaacgaaaattcaaacgttactg
tccgtctcgggacaatgagccctacgctaattggg
gtaaccgtggctgccgtcgtgagcgcaacgatcgg
cctcgtcattgtaatttccatcgtcaccagaaaca
tgtgcaccccgcaccgaaaattagacacggtctcg
caagacgacgaagaacgttcccaaactagaaggga
atcgcgaaaatttggacccatggttgcgtgcgaaa
taaacaagggggctgaccaggatagtgaacttgtg
gaactggttgcgattgttaacccgtctgcgctaag
ctcgcccgactcaataaaaatgtgattaagtctga
atgtggctctccaatcatttcgattctctaatctc
ccaatcctctcaaaaggggcagtatcggacacgga
ctgggaggggcgtacacgatagttatatggtacag
cagaggcctctgaacacttaggaggagaattcagc
cggggagagcccctgttgagtaggcttgggagcat
attgcaggatgaacatgttagtgatagttctcgcc
tcttgtcttgcgcgcctaacttttgcgacgcgaca
cgtcctctttttggaaggcactcaggctgtcctcg
gggaagatgatcccagaaacgttccggaagggact
gtaatcaaatggacaaaagtcctgcggaacgcgtg
caagatgaaggcggccgatgtctgctcttcgccta
actattgctttcatgatttaatttacgacggagga
aagaaagactgcccgcccgcgggacccctgtctgc
aaacctggtaattttactaaagcgcggcgaaagct
taggtcaattccctggcattatgcccagtacatga
ccttatgggactttcctacttggcagtacatctac
gtattagtcatcgctattaccatggtgatgcggtt
ttggcagtacatcaatgggcgtggatagcggtttg
actcacggggatttccaagtctccaccccattgac
gtcaatgggagtttgttttggcaccaaaatcaacg
ggactttccaaaatgtcgtaacaactccgccccat
tgacgcaaatgggcggtaggcgtgtacggtgggag
gtctatataagcagagctcgtttagtgaaccgtca
gatcgcctggagacgccatccacgctgttttgacc
tccatagaagacaccgggcgcgccggatctatgac
aaacctgcaagatcaaacccaacagattgttccgt
tcatacggagccttctgatgccaacaaccggaccg
gcgtccattccggacgacaccctggagaagcacac
tctcaggtcagagacctcgacctacaatttgactg
tgggggacacagggtcagggctaattgtctttttc
cctggattccctggctcaattgtgggtgctcacta
cacactgcagagcaatgggaactacaagttcgatc
agatgctcctgactgcccagaacctaccggccagc
tacaactactgcagactagtgagtcggagtctcac
agtgaggtcaagcacactccctggtggcgtttatg
cactaaacggcaccataaacgccgtgaccttccaa
ggaagcctgagtgaactgacagatgttagctacaa
tgggttgatgtctgcaacagccaacatcaacgaca
aagttgggaatgtcctggtaggggaaggggtcact
gtcctcagcctacccacatcatatgatcttgggta
tgtgaggcttggtgaccccattcccgctatagggc
ttgacccaaaaatggtagctacatgcgacagcagt
gacaggcccagagtctacaccataactgcagccga
tgattaccaattctcatcacagtaccaaccaggtg
gggtaacaatcacactgttctcagccaacattgat
gctatcacaagcctcagcattgggggagagctcgt
gtttcaaacaagcgtccaaggccttgtactgggcg
ccaccatctaccttataggctttgatgggactgcg
gtaatcaccagagctgtggccgcagataatgggct
gacggccggcaccgacaatcttatgccattcaatc
ttgtcattccaaccaatgagataacccagccgatc
acatccatcaaactggagatagtgacctccaaaag
tggtggtcaggcaggggatcagatgtcatggtcgg
caagtgggagcctagcagtgacgatccatggtggc
aactatccaggggccctccgtcccgtcacactagt
agcctacgaaagagtggcaacaggatccgtcgtta
cggtcgctggggtgagtaacttcgagctgatccca
aatcctgaactagcaaagaacctggttacagaata
cggccgatttgacccaggagccatgaactacacaa
aattgatactgagtgagagggaccgtcttggcatc
aagaccgtctggccaacaagggagtacactgattt
tcgtgagtacttcatggaggtggccgacctcaact
ctcccctgaagattgcaggagcatttggcttcaaa
gacataatccgggctataaggaggtaagatccata
attgattgacgggagatgggggaggctaactgaaa
cacggaaggagacaataccggaaggaacccgcgct
atgacggcaataaaaagacagaataaaacgcacgg
gtgttgggtcgtttgttcataaacgcggggttcgg
tcccagggctggcactctgtcgataccccaccgag
accccattggggccaatacgcccgcgtttcttcct
tttccccaccccaccccccaagttcgggtgaaggc
ccagggctcgcagccaacgtcggggcggcaggccc
tgccatagccactggccccgtgggttagggacggg
gtcccccatggggaatggtttatggttcgtggggg
ttattattttgaagcttgcctccgattctagcatt
acatagccggtcagtagatcctgccattcggtagc
gcaaccggctacatcttcaaacagtctcacaataa
atgcatctctcgttcctgccaatccggaaccgggc
ataccactcccgcctgccgatttaattctcacaat
tgggcgatgccggcggggcaaaacgaatgtggatt
tggcaaaccgacacaggtctgctgtacggactaat
atgggcacacccacatcattcttcagatgctccat
gcattgttctatgagaaagatccatagggtggagg
cagcgtcacgagatcgcccaggcaatcgatcgcat
tcgtctagtaaagtgacgagagttatcatgcacac
acccatgcccacgccttccgaataactggagctgt
ggaagatcggaaacgtctttttgactgccggtctc
gtactactttcgcacaggtgtatacccggacgcgt
actatatattttatatcatccaacgtccgaaatta
catacgtggcggcgatggaagtagatgttgagtct
tcgaaagtaagtgcctcgaatatgggtattgtctg
tgaaaatatcgaaagcggtacgacggttgcagaac
cgtcgatgtcgccagatactagtaacaatagcttc
gataacgaagacttccgtgggcctgaatacgatgt
ggagataaataccagaaaatctgctaatcttgatc
gtatggaatcttcgtgccgtgaacaacgagcggcg
tgcgaacttcgaaagtgttcgtgtcctacgtctgc
cgtgcgcatgcaatacagtattctttcatctctcg
ctccgggttcagagggtcatgtatatatatgtact
agatacggggacgcggaccaaaaaaaatgcatagt
gaaggcagtcgttggaggaaagaatcccgggaggg
aagtggatattttaaaaaccatctcacataaatca
attataaaattaatccatgcctataaatggaaaaa
tgttgtgtgtatggcaatgcgtgtatatcgttatg
atcttttcacatatattgacggagtcggccctatg
ccccttcaacagatgatctatattcaacgtggact
actagaggcgctagcatacatacatgaaaggggca
tcattcaccgagacgtaaagacggagaatatattc
ttggataatcacgaaaatgcagttttgggtgactt
cggtgctgcatgccaactaggagattgtatagata
cgccccaatgttacggttggagcggaactgtggaa
acaaattcgccggaattatctgcacttgatccgta
ttgcacaaaaacagatatttggagtgccggattgg
ttctatatgagatggcaattaaaaatgtaccattg
tttagtaagcaggtgaaaagttcgggatctcagct
gagatccataatacggtgcatgcaagtgcatgaac
tggagtttccccgcaacgattctaccaacctctgt
aaacatttcaaacaatatgcggttcgtgtacgacc
gccttataccattcctcgagttataagaaatgggg
ggatgccaatggatgttgaatatgtcatttctaaa
atgcttacgtttgaccaggagttcagaccttctgc
taaggaaatattgaatatgcccctatttactaagg
cgccgattaacctgcttaatatcacaccctctgac
agtgtctaacggtatacaggcgggagcgggtcgtg
gcgtcatcatcaccacttgagaatttatattttga
attgttgattgataaattaacctgattcattgaga
actgaaacgccatattggtttcttggatatgtcta
caacaattagttaaattgctatgttctactgcgag
taacatttgataagttgtaagagacgggcgactca
tgtcgaagttgacgaatataaagtacataacgtgt
ttagaatacccagaatccgaatagtccgcgggggc
gtcttctcgcgtgagtaccaaatactgagttgaac
ttgaaaatgctaaatctgtgacactctttgtgtga
tgattattgtcaccacttcgaagatggcttcgaca
ttcatgatgttctggtgtttgtttggaatcgtaat
agcgcttgtttcgtccaagtctgacaacaaagaaa
atctgaagaattatatcacggataagtcaaccaat
attagaatacccacgccattatttgtatcaacgga
aaactcttatcccacaaaacatgtaatctacgatg
aaaactgtggcttcgctgtactcaatcctataagt
gaccccaaatatgtccttttgagccagcttctaat
gggaaggcgcaaatatgatgcgacggtcgcgtggt
ttgttctcggtaaaatgtgtgccagattaatatat
ttgcgcgaattttataactgctcgacaaatgagcc
ttttggcacatgttctatgagctctcctggatggt
gggacaggcgctacgtctcaaccagtttcatttct
cgcgacgaattacagctggtttttgcagcgccgtc
ccgagaattagatggtttatatacgcgcgtagtag
ttgtcaacggggactttactacggccgatataatg
tttaatgttaaagtggcatgtgccttttcaaagac
tggaatagaagatgatacattatgcaaaccctttc
atttctttgccaatgcaacattgcacaatttaacc
atgattagatcggtaactcttcgagcgcacgaaag
ccatttaaaggaatgggtggcacggagaggtggta
acgtccctgcagtgctacttgagtctaccatgtat
catgcatccaatctgcctagaaatttcagggattt
ctacataaagtctccagatgattataagtataatc
acctagatgggccatctgtaatgctcatcactgac
agacctagtgaagatttggatgggaggctcgttca
ccaaagtgacatttttactactacaagtcctataa
aacaggtccggtatgaagagcatcagtcacataca
aagcagtatcctgtaaacaaaatacaagctataat
ttttttgatagggttaggctcgttcattggaagca
tattcgtagttttggtagtatggattatacgcaga
tattgcaatggagcgcggagtgggggaacgccccc
cagtcctcgccggtatgtgtataccaggctatgat
cacgtgtgaaacttgggcggacctgtatcatatgt
acaccgtccctattcgtttatagccagtacgtgtt
atctgcacatagaggaacatgtgtcatactgggat
cgcatgcatggtatgtgtgactctaatattattct
gtatcataataaaaacacagtgcatggtatataga
ggatcgctggtaagcactacggtagaccaatcggc
tcagattgcattctttggcatcgataccgttgtta
atttatatggcaaagtcttgttcatgggagatcag
tatttggaggaaatatactctggaacgatggaaat
actcaaatggaatcaagctaaccgctgctattcta
ttgcgcatgcaacatattacgccgactgtcctata
atcagttctacggtattcagaggatgccgggacgc
cgttgtttatactaggccccacagcagaattc
SEQ ID NO 18: 484-1050-2641-10859
(mCMV IEpro-VP2-SV40pA/ILT/HVT)
(15252 bp)
(IBDV + ILT gene cassettes
in HVT Ascl fragment.) Virus no.
HVT/IBDV/ILT 484
ggcgcgccactggagaacggcatgaccgcaaaagg
cgttgtagagatcgatcccacgaactctcaggcga
tcgtgtcagtcgccataaacagcgacgatcgtctc
caggatctgaacggttttcttctcaacgatcatca
gtatatgaggaactgaacctgatatttagccgagg
gaaacgcaggttaaaaaccctatcaagcgattgcg
attttcgcgtatctagtaaaaatagatgggcttcg
gtactagccttcgccgccaactctgaatatgccct
tcgtggacctcatataacatggcattgtttgttgg
atgcggggccggaattaagaagaacattcgaaata
cgagcaaaaatttcggccctggcatgtgctgcgcg
agaatcggtacttcggggagaaagttttatcggag
ctttgggtagtgcagaggaaactctatcttggttg
aaaatgcatgcgaccctgcacttgattctggttaa
ccacgatccaatttttaagacggctggcgcggtcc
tagataacctccgcttaaaactagccccaatattg
atgtgcagatataacacagaaaaacgatcaatgga
agacatgctacggcggtcatctcccgaagacatca
ccgattccctaacaatgtgcctgattatgttatcg
cgcattcgtcgtaccatgcgcaccgcaggaaataa
atatagctatatgatagatccaatgaatcgtatgt
ctaattacactccaggcgaatgtatgacaggtata
ttgcgatatattgacgaacatgctagaaggtgtcc
tgatcacatatgtaatttgtatatcacatgtacac
ttatgccgatgtatgtgcacgggcgatatttctat
tgtaattcatttttttgttagtaaactaccacagg
ctgtccggaaatctaagttaatgaataaagtagat
ggttaatactcattgcttagaattggactactttt
aattctctttaatgttcgtattaaataaaaacatc
tttaataaacttcagcctcttcgcttattgtagaa
attgagtattcaaaatcatgttcaaagccgtcttc
ggagagtgtactcgccacggtggttggaacatcac
tatgtctacacgtcaaatttaagcacgtcaggtct
gtcgaggacaagaaatggttaactagtgtttcaat
tattcttataaacgttaagcattgtaagccccccg
gccgtccgcagcaacaatttactagtatgccgtgg
gctccgggactatcacggatgtccaattcgcacat
gcatataatttttctagggtctctcatttcgagaa
atcttcggggatccatcagcaatgcgggctgtagt
cccgattcccgtttcaaatgaaggtgctccaacac
ggtcttcaaagcaaccggcataccagcaaacacag
actgcaactccccgctgcaatgattggttataaac
agtaatctgtcttctggaagtatatttcgcccgac
aatccacggcgcccccaaagttaaaaaccatccat
gtgtatttgcgtcttctctgttaaaagaatattga
ctggcattttcccgttgaccgccagatatccaaag
tacagcacgatgttgcacggacgactttgcagtca
ccagccttcctttccacccccccaccaacaaaatg
tttatcgtaggacccatatccgtaataaggatggg
tctggcagcaaccccataggcgcctcggcgtggta
gttctcgaggccttaagcttaaggatcccccaact
ccgcccgttttatgactagaaccaatagtttttaa
tgccaaatgcactgaaatcccctaatttgcaaagc
caaacgccccctatgtgagtaatacggggactttt
tacccaatttcccacgcggaaagccccctaataca
ctcatatggcatatgaatcagcacggtcatgcact
ctaatggcggcccatagggactttccacatagggg
gcgttcaccatttcccagcataggggtggtgactc
aatggcctttacccaagtacattgggtcaatggga
ggtaagccaatgggtttttcccattactggcaagc
acactgagtcaaatgggactttccactgggttttg
cccaagtacattgggtcaatgggaggtgagccaat
gggaaaaacccattgctgccaagtacactgactca
atagggactttccaatgggtttttccattgttggc
aagcatataaggtcaatgtgggtgagtcaataggg
actttccattgtattctgcccagtacataaggtca
atagggggtgaatcaacaggaaagtcccattggag
ccaagtacactgcgtcaatagggactttccattgg
gttttgcccagtacataaggtcaataggggatgag
tcaatgggaaaaacccattggagccaagtacactg
actcaatagggactttccattgggttttgcccagt
acataaggtcaatagggggtgagtcaacaggaaag
ttccattggagccaagtacattgagtcaataggga
ctttccaatgggttttgcccagtacataaggtcaa
tgggaggtaagccaatgggtttttcccattactgg
cacgtatactgagtcattagggactttccaatggg
ttttgcccagtacataaggtcaataggggtgaatc
aacaggaaagtcccattggagccaagtacactgag
tcaatagggactttccattgggttttgcccagtac
aaaaggtcaatagggggtgagtcaatgggtttttc
ccattattggcacgtacataaggtcaataggggtg
agtcattgggtttttccagccaatttaattaaaac
gccatgtactttcccaccattgacgtcaatgggct
attgaaactaatgcaacgtgacctttaaacggtac
tttcccatagctgattaatgggaaagtaccgttct
cgagccaatacacgtcaatgggaagtgaaagggca
gccaaaacgtaacaccgccccggttttcccctgga
aattccatattggcacgcattctattggctgagct
gcgttctacgtgggtataagaggcgcgaccagcgt
cggtaccgtcgcagtcttcggtctgaccaccgtag
aacgcagagctcctcgctgcaggcggccgctctag
aactcgtcgatcgcagcgatgacaaacctgcaaga
tcaaacccaacagattgttccgttcatacggagcc
ttctgatgccaacaaccggaccggcgtccattccg
gacgacaccctggagaagcacactctcaggtcaga
gacctcgacctacaatttgactgtgggggacacag
ggtcagggctaattgtctttttccctggattccct
ggctcaattgtgggtgctcactacacactgcagag
caatgggaactacaagttcgatcagatgctcctga
ctgcccagaacctaccggccagctacaactactgc
agactagtgagtcggagtctcacagtgaggtcaag
cacactccctggtggcgtttatgcactaaacggca
ccataaacgccgtgaccttccaaggaagcctgagt
gaactgacagatgttagctacaatgggttgatgtc
tgcaacagccaacatcaacgacaaaattgggaatg
tcctggtaggggaaggggtcactgtcctcagccta
cccacatcatatgatcttgggtatgtgaggcttgg
tgaccccattcccgctatagggcttgacccaaaaa
tggtagctacatgcgacagcagtgacaggcccaga
gtctacaccataactgcagccgatgattaccaatt
ctcatcacagtaccaaccaggtggggtaacaatca
cactgttctcagccaacattgatgctatcacaagc
ctcagcattgggggagagctcgtgtttcaaacaag
cgtccaaggccttgtactgggcgccaccatctacc
ttataggctttgatgggactgcggtaatcaccaga
gctgtggccgcagataatgggctgacggccggcac
cgacaatcttatgccattcaatcttgtcattccaa
ccaatgagataacccagccaatcacatccatcaaa
ctggagatagtgacctccaaaagtggtggtcaggc
aggggatcagatgtcatggtcggcaagtgggagcc
tagcagtgacgatccatggtggcaactatccaggg
gccctccgtcccgtcacactagtagcctacgaaag
agtggcaacaggatccgtcgttacggtcgctgggg
tgagtaacttcgagctgattccaaatcctgaacta
gcaaagaacctggttacagaatacggccgatttga
cccaggagccatgaactacacaaaattgatactga
gtgagagggaccgtcttggcatcaagaccgtctgg
ccaacaagggagtacactgattttcgtgagtactt
catggaggtggccgacctcaactctcccctgaaga
ttgcaggagcatttggcttcaaagacataatccgg
gctataaggaggtagatccagacatgataagatac
attgatgagtttggacaaaccacaactagaatgca
gtgaaaaaaatgctttatttgtgaaatttgtgatg
ctattgctttatttgtaaccattataagctgcaat
aaacaagttaacaacaacaattgcattcattttat
gtttcaggttcagggggaggtgtgggaggtttttt
cggatcctctagagtcgacggcagagtcgcagacg
cccctattggacgtcaaaattgtagaggtgaagtt
ttcaaacgatggcgaagtaacggcgacttgcgttt
ccaccgtcaaatctccctatagggtagaaactaat
tggaaagtagacctcgtagatgtaatggatgaaat
ttctgggaacagtcccgccggggtttttaacagta
atgagaaatggcagaaacagctgtactacagagta
accgatggaagaacatcggtccagctaatgtgcct
gtcgtgcacgagccattctccggaaccttactgtc
ttttcgacacgtctcttatagcgagggaaaaagat
atcgcgccagagttatactttacctctgatccgca
aacggcatactgcacaataactctgccgtccggcg
ttgttccgagattcgaatggagccttaataatgtt
tcactgccggaatatttgacggccacgaccgttgt
ttcgcataccgctggccaaagtacagtgtggaaga
gcagcgcgagagcaggcgaggcgtggatttctggc
cggggaggcaatatatacgaatgcaccgtcctcat
ctcagacggcactcgcgttactacgcgaaaggaga
ggtgcttaacaaacacatggattgcggtggaaaac
ggtgctgctcaggcgcagctgtattcactcttttc
tggacttgtgtcaggattatgcgggagcatatctg
ctttgtacgcaacgctatggaccgccatttatttt
tgaggaatgctttttggactatcgtactgctttct
tccttcgctagccagagcaccgccgccgtcacgta
cgactacattttaggccgtcgcgcgctcgacgcgc
taaccataccggcggttggcccgtataacagatac
ctcactagggtatcaagaggctgcgacgttgtcga
gctcaacccgatttctaacgtggacgacatgatat
cggcggccaaagaaaaagagaaggggggccctttc
gaggcctccgtcgtctggttctacgtgattaaggg
cgacgacggcgaggacaagtactgtccaatctata
gaaaagagtacagggaatgtggcgacgtacaactg
ctatctgaatgcgccgttcaatctgcacagatgtg
ggcagtggactatgttcctagcacccttgtatcgc
gaaatggcgcgggactgactatattctcccccact
gctgcgctctctggccaatacttgctgaccctgaa
aatcgggagatttgcgcaaacagctctcgtaactc
tagaagttaacgatcgctgtttaaagatcgggtcg
cagcttaactttttaccgtcgaaatgctggacaac
agaacagtatcagactggatttcaaggcgaacacc
tttatccgatcgcagacaccaatacacgacacgcg
gacgacgtatatcggggatacgaagatattctgca
gcgctggaataatttgctgaggaaaaagaatccta
gcgcgccagaccctcgtccagatagcgtcccgcaa
gaaattcccgctgtaaccaagaaagcggaagggcg
caccccggacgcagaaagcagcgaaaagaaggccc
ctccagaagactcggaggacgacatgcaggcagag
gcttctggagaaaatcctgccgccctccccgaaga
cgacgaagtccccgaggacaccgagcacgatgatc
caaactcggatcctgactattacaatgacatgccc
gccgtgatcccggtggaggagactactaaaagttc
taatgccgtctccatgcccatattcgcggcgttcg
tagcctgcgcggtcgcgctcgtggggctactggtt
tggagcatcgtaaaatgcgcgcgtagctaatcgag
cctagaataggtggtttcttcctacatgccacgcc
tcacgctcataatataaatcacatggaatagcata
ccaatgcctattcattgggacgttcgaaaagcatg
gcatcgctacttggaactctggctctccttgccgc
gacgctcgcacccttcggcgcgatgggaatcgtga
tcactggaaatcacgtctccgccaggattgacgac
gatcacatcgtgatcgtcgcgcctcgccccgaagc
tacaattcaactgcagctatttttcatgcctggcc
agagaccccacaaaccctactcaggaaccgtccgc
gtcgcgtttcggtctgatataacaaaccagtgcta
ccaggaacttagcgaggagcgctttgaaaattgca
ctcatcgatcgtcttctgtttttgtcggctgtaaa
gtgaccgagtacacgttctccgcctcgaacagact
aaccggacctccacacccgtttaagctcactatac
gaaatcctcgtccgaacgacagcgggatgttctac
gtaattgttcggctagacgacaccaaagaacccat
tgacgtcttcgcgatccaactatcggtgtatcaat
tcgcgaacaccgccgcgactcgcggactctattcc
aaggcttcgtgtcgcaccttcggattacctaccgt
ccaacttgaggcctatctcaggaccgaggaaagtt
ggcgcaactggcaagcgtacgttgccacggaggcc
acgacgaccagcgccgaggcgacaaccccgacgcc
cgtcactgcaaccagcgcctccgaacttgaagcgg
aacactttacctttccctggctagaaaatggcgtg
gatcattacgaaccgacacccgcaaacgaaaattc
aaacgttactgtccgtctcgggacaatgagcccta
cgctaattggggtaaccgtggctgccgtcgtgagc
gcaacgatcggcctcgtcattgtaatttccatcgt
caccagaaacatgtgcaccccgcaccgaaaattag
acacggtctcgcaagacgacgaagaacgttcccaa
actagaagggaatcgcgaaaatttggacccatggt
tgcgtgcgaaataaacaagggggctgaccaggata
gtgaacttgtggaactggttgcgattgttaacccg
tctgcgctaagctcgcccgactcaataaaaatgtg
attaagtctgaatgtggctctccaatcatttcgat
tctctaatctcccaatcctctcaaaaggggcagta
tcggacacggactgggaggggcgtacacgatagtt
atatggtacagcagaggcctctgaacacttaggag
gagaattcagccggggagagcccctgttgagtagg
cttgggagcatattgcaggatgaacatgttagtga
tagttctcgcctcttgtcttgcgcgcctaactttt
gcgacgcgacacgtcctctttttggaaggcactca
ggctgtcctcggggaagatgatcccagaaacgttc
cggaagggactgtaatcaaatggacaaaagtcctg
cggaacgcgtgcaagatgaaggcggccgatgtctg
ctcttcgcctaactattgctttcatgatttaattt
acgacggaggaaagaaagactgcccgcccgcggga
cccctgtctgcaaacctggtaattttactaaagcg
cggcgaaagcttcccgggttaattaaggccctcga
ggatacatccaaagaggttgagtattctctctaca
cttcttgttaaatggaaagtgcatttgcttgttct
tacaatcggcccgagtctcgttcacagcgcctcgt
tcacacttaaaccacaaatagtctacaggctatat
gggagccagactgaaactcacatatgactaatatt
cgggggtgttagtcacgtgtagcccattgtgtgca
tataacgatgttggacgcgtccttattcgcggtgt
acttgatactatggcagcgagcatgggatattcat
cctcgtcatcgttaacatctctacgggttcagaat
gtttggcatgtcgtcgatcctttgcccatcgttgc
aaattacaagtccgatcgccatgaccgcgataagc
ctgtaccatgtggcattagggtgacatctcgatca
tacattataagaccaacgtgcgagtcttccaaaga
cctgcacgccttcttcttcggattgtcaacgggtt
cttcagaatctatgcccatatctggcgttgagacc
attgtgcgtttaatgaacaataaagcggcatgcca
tggaaaggagggctgcagatctccattttctcacg
ccactatcctggacgctgtagacgataattatacc
atgaatatagagggggtatgtttccactgccactg
tgatgataagttttctccagattgttggatatctg
cattttctgctgccgaacaaacttcatcgctatgc
aaagagatgcgtgtgtacacgcgccggtggagtat
acgggaaactaaatgttcatagaggtctttgggct
atatgttattaaataaaataattgaccagtgaaca
atttgtttaatgttagtttattcaatgcattggtt
gcaaatattcattacttctccaatcccaggtcatt
ctttagcgagatgatgttatgacattgctgtgaaa
attactacaggatatatttttaagatgcaggagta
acaatgtgcatagtaggcgtagttatcgcagacgt
gcaacgcttcgcatttgagttaccgaagtgcccaa
cagtgctgcggttatggtttatgcgcacagaatcc
atgcatgtcctaattgaaccatccgatttttcttt
taatcgcgatcgatgtttgggcaactgcgttattt
cagatctaaaaaatttaccctttatgaccatcaca
tctctctggctcataccccgcttggataagatatc
atgtagattccgccctaagaaatgcaaactaacat
tattgtcggttccatatacacttccatcttgtcct
tcgaaaataacaaactcgcgcaatagaccgtccgt
acatgcatggccgatgtgtgtcaacatcattggtc
tgctagatcccgatgggacgaatcgtacagtcgtc
gctccagcattggcaaaaatccccagataccctcc
atgcggcaaatctaaattgcgaccccgaagagact
gcaccaaagtcttatcgacgcacgctgattttttt
gaacagcgggagcccattatcttcagtggagcgta
gacgggcgaggctaattatgtgacatagcaacact
gcatgtatgtttttataaatcaataagagtacata
atttattacgtatcatttccgtttgtaatatactg
tatacatcatccacactattagtcagcactagcgc
gcgggcgcacgttacaatagcagcgtgcccgttat
ctatattgtccgatatttacacataacatttcatc
gacatgattaaatacctaagtactgcacacagatg
tttaatgtatatcgtcatataaattatatcgctag
gacagacccaaacgacctttatcccaaacagtcag
atcctcttctcaagtgtcgatttctgttatggaat
atgcataccctggcccagaaattgcacgcacgagc
gtagtgaatgcgtcattggttttacatttaaaggc
taaatgcacaaattctttagacgacagcacatcgt
taaatagcatctctagcgttcttatgaatgctaag
cattggagtcctcctggtcggccacaataacagct
gagtatcataccctgagctccggggttgtcgcaca
tagcggattcgtataaacataggattttccgcgaa
tccatcagttgcaaaaatctgttaggctccatcaa
caacgctggatttacttcagatccacgcgtaaagt
aatggtgctcgaataccgtttttagagttgtcggc
atttcaaggaacaaagaattcatttcttcattgca
acgacgcgccagaaatcccaagacctctttgggta
gtatgttcttgcctataaaacacggcgttccaagt
gccaggaaccacgcatgtgttactgttggggcgta
ttcagaaataaagcggggtttatgcggcttttgaa
gctcggatatccaaagtatcgcttgctgatgaacg
agcgatgtagctgttacaaaacctcctttccatcc
tccagtcaacataatatttatcggcctacctatgt
ccgtaataagtattggtcgggcaattattccgtat
gaggtcttgcaggaataagctcttagggacagcca
gcttggatatggtgcgaaacagaccttctcggctt
cagaatgtcgctccgcagtctcttcgtgtcggtgc
atcttagatccaccatcaatgtgtgcagcattgac
tcccgcccgtcgaatattccttttgttacgatgca
gtaatgagcacgatcatgggcggggcgatgacgtt
ctatttgcatgtctgcgaacaatttgcgtcagtca
tacagctatggagtgggccatttctggccgtcaac
ttaaaaacgcgaaccgcagacatatgtatttgcat
gcaaagacgtatcttcgtatttctgggcatcttca
aatgctctggccaatatggcaatgaatttggattc
gtttgacgccgatggtatgcagtgcaaatgtgcca
atagcccacatccgaaaaagttatttgtcatacaa
gcaggtgttaagtagcaatcacataaaggcaccag
acgcctcatggcatcataatgaatagctccttctc
cccactggaaccactgacaaaatctgcgagtatat
tccgcaaaccacattttatttctcatagaaactac
cctaaatccttttaacgggaagaagaatcctagat
agtgcttgaagtcatgactgttactgctgcaataa
cactgtatattatttataaattccgtttgtctagg
tatctgatgtaggcattccgatccctttactattg
cgtcttcacgaccaaatgggaatgcgccaaaatcc
ccacacctcatcaccctggaggcagattgtgtatt
attaatatccgccgattgaagcacaaaacggtacg
gtactgttcctaattctggtatagattctatggtc
aaaagtctgcatatccccgacattgccatgagatc
acacagtccaagtagcatgtttattgagtcactca
gactgtcaacgtccctcgccgcaccaccaatcgaa
aataaagtatctacgcaagttatagctccgcattt
tctatcgctagcagcaatcgcgacgcaaaacataa
aggccatgttgggatttgaactctctggggggctt
gttatcttctgcaccgtcgcagtcgcagttttccg
aaatttatgtctaatatattttccggccgtgctcc
aatcggccgaaaagaatctgcgtattaccagactc
attgacgggccgataaagaccataaaacaaaattc
ctgtgcactccctcctccagttttgccatcgtcca
agtcccgtaactttttttgcgtttcgaggagcaag
cgttcgttatccctacccacacttgttttccaccg
ttttcttattataagcggttgtatcgccaacgcgt
caccgcaggttgtcacatacagtgatggcatactt
gaacgtgcaacaacgcgctcgctttgcaaatctaa
gtcattgaccatcaaatcgcgttgagaggatagcc
aggcatcttttttcctagtatggtgacggtgcagc
caccccaactcagttcttgtaaaaaaagctattgg
cgggaatttatgttctgaggtgcattctatattta
tgagtccatcaaatgccattaaccagattcgtatt
ttttcgctcgacccggcatcactatggatacaata
cctttctatggcccatttcagctctcgaaccaacc
acacggacaattgactaacataagtatgatcttta
tcacagtcgcacccatctgagttatatttatggca
tccgagcgctcttactgtacggtcggatacaccca
tggtttttcctttatatagtcgggttatagtctgt
cgggtttggcggtagcacggagtagtttgattttt
aagaatcgaaaaccggcttggagagaccactgtcg
aatatttgtccgtatactctacacgtgagtgttgt
ccattcctaggtatattcatctgttcggatacctt
caattgctgttcaggcataaccttaaagcatatgt
tatgttgtacatcaaaacttggtgagttatgttcg
attgccgcgcataaagaatcgtacatgagcgtttc
tgctaacatactatctatattctcacacgcccctg
catatactgttcctattccaaattcacgttttgcc
ccatcggctatctgctcccaaaaagttgtaatata
ggtgccgctgggtgcgaaattttcatcagttgtat
tcctgataaactgaatcactttacataatttttgc
cacatatctgcgtgcagccatagtatcgaacccgt
gggctcggagacgacagtgcgtacaatgggtattt
tacctttccccaacaaaataatggtatacaagtta
ggtccgtacctagaccttaatgtttccaattcttc
tgaatcactgcactctcgtaggggagtaacggtaa
taatttcgtctctgagccccgttttgcgttgaaaa
ctaatcacattagataatgtgcaatcggtttcttt
tatccggatacatctaagtattatgacatcggtgg
tcattgtttccatcaacgaccatcttttacgatcg
cccatactactcatggacgttgtcggtgttgaaaa
atcaccagaattgcaacggatctctgggtaccatg
ctgctgatggaattggcggttttaattgttgtttc
agtctattattgctatctttggcggggttgaataa
tgtggggggagagtgattgcaggaatccgaatggg
tcaataaaacgaccgtgctccgttctgccggcgcc
gatccgattgaagctatatacttcgcttctctccc
cacttttccaatttgatccggaaataaaacggccc
cggacaacagtatcgtacgatccggatccggatcc
tgcttgcctacagaagaatcaacatctcgccccaa
tattctggtcaaaactggctcgctcatggcaacgc
ggacgtttcccccggtggccagtcttaatggttaa
tgttcttttcggcaatcttatacatcagcgggttg
cgtgaatactggtcacagttcagtcatttactaca
caccagcaatacgacgacggacagtaccgtcccga
cgaacgcgacgcccaaaattgctatcgcgaccgcg
tccgaggcgatgtcgtacgggcggtgcggggttgg
atcctcggcaaagagatcctcgtaattcggcggtg
ggagcggagggtaaagacgcgggtggggatctccc
tccggaccgcgcgccgggcgcggttcgaaaatgct
ttccgcctcgctcagtgtcaacgccaagtattcgg
gcgggctgggggccggaatatctcccgcgacttct
tctatcggcgcggaattggagtcgcggtcgtggcg
cgcttctagcgtcgtcaacggaagtccattttcgg
ggtctcccggtgggcgttcagcgtccatcgtcgta
tatgctctaacacacgtctcgctatattaaaaaaa
agaagagtatcggtcagtgtcgagtgtcgccgaca
atgtcgcgagttctcggcgatttaatttttggaac
tgctccctatgaatcccgtaactgtagcgcccgcg
cagaaagccgccatcagaccaactacgtgtctgtt
cgatgtttgcccgccgatcgctttaccgattaagg
ttccggcgagaaatgacatgctcgatccaagaaca
aagtttttcgcggtaaacaacaacatagttaccgt
gcgagatggagaaaccacatctcccgaattagtag
aggaaagcccgcgctgtcggtttggggacatatcg
atcttttttgtgtttttcctaggacccttttgcca
gatcgtacaaagtcgcgtcttatgagcggacgttc
ttactgcagctcggtaggagtggggcagggttaga
tttcgtcggcgtttcggcccccgtatgcgccgcgc
caccctcttcgccgagctctttatgcgcggtgggg
gtgagcgcttccggagttgcgatctccgatctcga
gccgcagcccggcggtgtctctttcagtggagcgt
tagcgccatcatgtggttcgtggcggtggaaaggc
tattatgtgttaggggagagaccacgtgatcggca
tgcaaatgagcaaggcgaacgcgtcagcgttcgca
ctgcgaaccaataatatatatattatactattggc
tttaggtgcgaacgtccggctagtccaatagcggg
gtcgcgtttcgtaccacgtgttatagaccgcccta
aactcgcactcgggggtccggccgcgcccagacag
ggcggagacgtgccacaggggctttaaaacaccgc
ttcgggcaccgttcatctcggcgcgcc
SEQ ID NO 19: SV40 polyadenylation
signal (199 bp)
agcttcagacatgataagatacattgatgagtttg
gacaaaccacaactagaatgcagtgaaaaaaatgc
tttatttgtgaaatttgtgatgctattgctttatt
tgtaaccattataagctgcaataaacaagttaaca
acaacaattgcattcattttatgtttcaggttcag
ggggaggtgtgggaggttttttcg

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all base sizes or amino acid sizes, and all molecular weight or molecular mass values, given for nucleic acids or polypeptides are approximate, and are provided for description.

1.-17. (canceled)

18. A method for the preparation of a vaccine, the method comprising:
a. infecting a culture of chicken embryo fibroblast cells (CEFs) with a recombinant herpesvirus of turkeys (rHVT), wherein the rHVT comprises a first heterologous nucleic acid located in a first nonessential site in the rHVT genome and a second heterologous nucleic acid located in a second nonessential site in the rHVT genome; wherein the first heterologous nucleic acid comprises both a nucleotide sequence that encodes an Infectious Laryngotracheitis Virus glycoprotein D (ILTV gD) and a nucleotide sequence that encodes an Infectious Laryngotracheitis Virus glycoprotein I (ILTV gI); wherein the second heterologous nucleic acid comprises a nucleotide sequence that encodes an Infectious Bursal Disease Virus viral protein 2 (IBDV VP2); wherein the first nonessential site and the second nonessential site are either the same or different; and wherein when the first nonessential site and the second nonessential site are different, one is the US2 site and the other is the UL54.5 site;
b. harvesting the infected CEFs;
c. optionally, obtaining the rHVT from the CEFs; and
d. admixing the harvested infected CEFs from step b., or the obtained rHVT from step c., with a pharmaceutically acceptable carrier.

19. The method of claim 18, wherein in the rHVT the first nonessential site and the second nonessential site are the US2 site.

20. The method of claim 18, wherein in the rHVT the nucleotide sequence encoding the ILTV gD protein is operatively under the control of a first promoter, the nucleotide sequence encoding the ILTV gI protein is operatively under the control of a second promoter, and the nucleotide sequence encoding the IBDV VP2 protein is operatively under the control of a third promoter.

21. The method of claim 20, wherein in the rHVT the first promoter, the second promoter, and the third promoter are all different.

22. The method of claim 21, wherein in the rHVT the first promoter is the endogenous ILTV gD promoter, the second promoter is the endogenous ILTV gI promoter, and the third promoter is selected from the group consisting of the murine cytomegalovirus immediate early 1 gene (mCMV-IE1) promoter, the human cytomegalovirus immediate early 1 gene (hCMV-IE1) promoter, and the chicken β-actin promoter.

23. The method of claim 18, wherein step c. further comprises sonification of the CEFs to obtain the rHVT.

24. The method of claim 18, further comprising lyophilizing the rHVTs obtained from step c. with the pharmaceutically acceptable carrier.

25. The method of claim 18, further comprising cryopreserving the harvested CEFs with the pharmaceutically acceptable carrier.

26. A method for the preparation of a vaccine, the method comprising: infecting a culture of CEFs with a rHVT that comprises a recombinant nucleic acid comprising in 5′ to 3′ direction in the following order:
(i) a murine cytomegalovirus immediate early 1 (mCMV-IE1) promoter;
(ii) a coding sequence for Infectious Bursal Disease Virus viral protein 2 (IBDV VP2);
(iii) a transcription terminator sequence;
(iv) an Infectious Laryngotracheitis Virus glycoprotein D (ILTV gD) promoter;
(v) a coding sequence for the ILTV gD protein;
(vi) an Infectious Laryngotracheitis Virus glycoprotein I (ILTV gI) promoter; and
(vii) a coding sequence for the ILTV gI protein.

27. The method of claim 26, wherein the recombinant nucleic acid comprises the nucleotide sequence of SEQ ID NO: 15.

28. The method of claim 26, wherein in the rHVT the nonessential insertion site is selected from the group consisting of US2 and UL54.5.

29. A chicken embryo fibroblast (CEF) cell, the CEF comprising a recombinant herpesvirus of turkeys (rHVT) comprising a nucleotide sequence that encodes an Infectious Laryngotracheitis Virus glycoprotein D (ILTV gD), a nucleotide sequence that encodes an Infectious Laryngotracheitis Virus glycoprotein I (ILTV gI), and a nucleotide sequence that encodes an Infectious Bursal Disease Virus viral protein 2 (IBDV VP2); wherein the nucleic acid sequence is located in a nonessential site in the rHVT genome; and wherein the nonessential insertion site is selected from the group consisting of the US2 site and the UL54.5 site.


SEQUENCE LISTING (19 sequences)

SEQ ID NO: 1 - DNA, 1134 bp, Infectious laryngotracheitis virus
  source 1..1134: mol_type = genomic DNA
atggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaa

SEQ ID NO: 2 - protein, 377 aa, Infectious laryngotracheitis virus
MDRHLFLRNAFWTIVLLSSFASQSTAAVTYDYILGRRALDALTIPAVGPYNRYLTRVSRGCDVVELNPISNVDDMISAAKEKEKGGPFEASVVWFYVIKGDDGEDKYCPIYRKEYRECGDVQLLSECAVQSAQMWAVDYVPSTLVSRNGAGLTIFSPTAALSGQYLLTLKIGRFAQTALVTLEVNDRCLKIGSQLNFLPSKCWTTEQYQTGFQGEHLYPIADTNTRHADDVYRGYEDILQRWNNLLRKKNPSAPDPRPDSVPQEIPAVTKKAEGRTPDAESSEKKAPPEDSEDDMQAEASGENPAALPEDDEVPEDTEHDDPNSDPDYYNDMPAVIPVEETTKSSNAVSMPIFAAFVACAVALVGLLVWSIVKCARS

SEQ ID NO: 3 - DNA, 1089 bp, Infectious laryngotracheitis virus
  source 1..1089: mol_type = genomic DNA
atggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtga

SEQ ID NO: 4 - protein, 362 aa, Infectious laryngotracheitis virus
MASLLGTLALLAATLAPFGAMGIVITGNHVSARIDDDHIVIVAPRPEATIQLQLFFMPGQRPHKPYSGTVRVAFRSDITNQCYQELSEERFENCTHRSSSVFVGCKVTEYTFSASNRLTGPPHPFKLTIRNPRPNDSGMFYVIVRLDDTKEPIDVFAIQLSVYQFANTAATRGLYSKASCRTFGLPTVQLEAYLRTEESWRNWQAYVATEATTTSAEATTPTPVTATSASELEAEHFTFPWLENGVDHYEPTPANENSNVTVRLGTMSPTLIGVTVAAVVSATIGLVIVISIVTRNMCTPHRKLDTVSQDDEERSQTRRESRKFGPMVACEINKGADQDSELVELVAIVNPSALSSPDSIKM

SEQ ID NO: 5 - DNA, 1362 bp, Infectious bursal disease virus
  source 1..1362: mol_type = genomic DNA
atgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtggccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaa

SEQ ID NO: 6 - protein, 453 aa, Infectious bursal disease virus
MTNLQDQTQQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQPGGVTITLFSANIDAITSLSIGGELVFQTSVQGLVLGATIYLIGFDGTAVITRAVAADNGLTAGTDNLMPFNLVIPTNEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRAIRR

SEQ ID NO: 7 - DNA, 527 bp, Infectious laryngotracheitis virus
  source 1..527: mol_type = genomic DNA
aaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgct

SEQ ID NO: 8 - DNA, 264 bp, Infectious laryngotracheitis virus
  source 1..264: mol_type = genomic DNA
tgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagc

SEQ ID NO: 9 - DNA, 3563 bp, Infectious laryngotracheitis virus
  source 1..3563: mol_type = genomic DNA
tcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtgattaagtctgaatgtggctctccaatcatttcgattctctaatctcccaatcctctcaaaaggggcagtatcggacacggactgggaggggcgtacacgatagttatatggtacagcagaggcctctgaacacttaggaggagaattcagccggggagagcccctgttgagtaggcttgggagcatattgcaggatgaacatgttagtgatagttctcgcctcttgtcttgcgcgcctaacttttgcgacgcgacacgtcctctttttggaaggcactcaggctgtcctcggggaagatgatcccagaaacgttccggaagggactgtaatcaaatggacaaaagtcctgcggaacgcgtgcaagatgaaggcggccgatgtctgctcttcgcctaactattgctttcatgatttaatttacgacggaggaaagaaagactgcccgcccgcgggacccctgtctgcaaacctggtaattttactaaagcgcggcgaa

SEQ ID NO: 10 - DNA, 1391 bp, Murine cytomegalovirus
  source 1..1391: mol_type = genomic DNA
aactccgcccgttttatgactagaaccaatagtttttaatgccaaatgcactgaaatcccctaatttgcaaagccaaacgccccctatgtgagtaatacggggactttttacccaatttcccacgcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcag

SEQ ID NO: 11 - DNA, 692 bp, Gallus gallus
  misc_difference 141..143: note = n is a, c, g, or t
  source 1..692: mol_type = genomic DNA
cgcgccggatcagatctccatggtcgaggtgagccccacgttctgcttcactctccccatctcccccccctccccacccccaattttgtatttatttattttttaattattttgtgcagcgatgggggcgggggggggggnnncgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgca

SEQ ID NO: 12 - DNA, 301 bp, Human cytomegalovirus
  source 1..301: mol_type = genomic DNA
ggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatag

SEQ ID NO: 13 - DNA, 55 bp, Feline herpesvirus
  source 1..55: mol_type = genomic DNA
caataaacatagcatacgttatgacatggtctaccgcgtcttatatggggacgac

SEQ ID NO: 14 - DNA, 370 bp, Herpes simplex virus
  source 1..370: mol_type = genomic DNA
gatccataattgattgacgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttga

SEQ ID NO: 15 - DNA, 14113 bp, synthetic construct
  misc_feature 1..14113: note = Herpesvirus of turkeys, Murine cytomegalovirus, Infectious bursal disease virus, Simian vacuolating virus 40, and Infectious laryngotracheitis virus
  source 1..14113: mol_type = other DNA
gaattccagactaaatgccccggcccaatttgtcaagtgtgcagtcacggaggcgtcgaccgtgtccccggcattaaacaggaaagcgttaaagtttttgaatgttaggtcacaggtacaaacataaatgtttgtacaaacaggtaacaggtacaaacataaatgccccggcataaatgtcccttacggcggatcgaaacgacattaggcatactcgggtaccattttgcattccgatcagcacggatgaaattaggcaggaatgcggtttatattatgcggcattggacaaacgatatggcattgattggcagtttatgaatgtcttcatgttgggcgtaaacggattcctattggttcagaagacaacgacgatatatttagagagaaaaagctacccagcataggataaacacacattgagcattgagagacataggtatcggtatggatgggaaaactacacacgtgaacaccaaacgacttatatactcgagcggtgatactactgagcaagaatgcactgcatctgagccactgaatgaagactgtgatgaaaatgtgaccatcgatggaattggagaagaatatgcgcagttcttcatgtccccgcaatgggtcccaaatctacatcgcttgagcgaggataccaaaaaggtataccgatgtatggtttccaacagactcaattattttccctattatgaggcgttcaggcggtctttgtttgatatgtatatgctaggtcggttggggcgtcgacttaagcgatctgactgggagactattatgcatctgtcaccaacgcaaagtcggcgtctacatagaactttaagatttgtggagcgtagaattatcccatctaacagttatatacgcacatcgggccacgttccgccttcgagggcacttccgacagatacgaatttaaagatggatgaataattaaattggaaagagtaactacattaatcgagcgtcatgacggcgtcccgtgaaaatgggaattttctactcgaaacaccgtgacatttgacagacctggaattgttattctgatatatagtgggtgtgtctggccggcaacatacataatgtgcatgcgaaaccactttttcagtgtacgctgacattgtgcaacacggaggggtagcatctacatacaatatatgttgattaatgattggagaaaaaactatgcagctcgccgatcatatggctaactcgccttcgtctatatggcggaccccgcgggaaaaatcgacgtaccatctgatttacaacaccagtaatgaacatgtcgcatccctgcccagatctgtgcgcccattggcgcggatcgttgtgaatgccgccgaaacacttcaggtcggtatgagagccgggaggccgccatcagcaggagtttggcgagaggtgtttgatagaatgatgacagccttccgtgaccacgagcctactgcgacatttaatgctgcaaatcccattagaaaaatggtcgagacagttctacagaataatgaagagcccccgcggacgcatgctgaaatgggtaatcgccttatgaacattatgtactggtgttgcttgggacacgcaggacaatgctcgatatggcagttgtacgagacgaatcaggccattttaagtttattagatgaagtggttatcggcacaacaaatcccttttgcaccctcgagcaatactggaagccattatgcaccgcaatcgccaacaaggggacctcatcgcttgttgaggatgccaaagtggccgagtacctggttagcatgcgcaaattgatataacataggcacgctctgatgttacagaccacaataccgcatacatttattgtaaggttgttaataaaggtttattctatgtaagactacaatactttcgacattgcttgtatacatattaaatactttctcaagttcctattacataaaatgggatctatcattacattcgttaagagtctggataattttactgtttgccagcttcgatcttggaacgtactgtggatagtgccttacttggaatcgtgaaaatttgaaacgtccattatttggatatcttccggttgtcccatatcccgccctggtaccgctcggataccttgcccgtatggattcgtattgacagtcgcgcaatcggggaccaacaacgcgtgggtccacactcattcggaaattttccgatgattctgaatatttattgccgctcgttacgagtcgttggacatatctgtaatacatttcttcttctgaaggatcgctgcacatttgatctatacattggccaggatgttcaagtctcagatgttgcattctggcacagcacaactttatggcatttccgatgtaatcgtccggcagccctgggggagttctatattcgcatattgggatggtaaggacaatagcagatctcgcaacctccagggaggctataataacgtttttaaaggatggatttctcataaaaatctgtcgcaaattacactgagaatatcctttactagcgccgattgagagcatcgtcgtccaattttctaaatggaaagaaaacaaggcgggcaagagtgttccaaacattttcattttcggcgaatctctcaaatcccatggcgtgcaattgattgcaaaattggcacttccgttcacgtttgtatctccaaactctaagacacttttaattgaaaaactacgttctagtgtggaaagaaacctataggcagaccatagaactatttgacaccacatatctttttgtatgtcaaactgaccatgatcgtatgttgctgaatgcactagggcaattcgctcgcgcgactccatacattgaataattccacacgtcagctcatcggttagcaaggtccagtagttgaagtcatttatttttccccgcggctggccaaatctacctctgggaatatccaagttgtcgaatatgatcgcaccggctctggtcatggtgaaggaactgtagcataaagacgcaggtatcataggggtaatatttttttattcactcacatactaaaagtaacgcatattagcaccatgtatgggctatcaattgacatttgcgtagcactacatcacgattatgtacaacataatgggacaacatatggcaagtagatgcaatttcctcacactagttgggtttatctactattgaattttcccctatctgtgatacacttgggagcctctacaagcatattgccatcatgtacgtttttatctactgtcttaacgcccatgggaacggaggcgtcgtcgtcatgtattggacggcaacataggcagcaacacaaattgcgtttaggtggggtgcatgtggactcgataccaagcccctgcagctggggaacgtctggtggagagccgataatttgatatacgcacgccatattactgtcgttgaagtacgccttatcttctatgttttcaaatttaggttcccaagtggacgtgagaagtgtttgtatctcacatggaatggcccaaggcattccagcccaggtgcctggtactttaatggcaaacaaacgttttggtagaggtattgattctattgcagttctgcagatatctgcagccccgagtatccacaggctatacgatacgttatcggaggcaagctgcggccgctctagaactagtggatcccccgggctgcagcccaatgtggaattcgcccttgcacattgttactcctgcatcttaaaaatatatcctgtagtaattttcacagcaatgtcataacatcatctcgctaaagaatgacctgggattggagaagtaatgaatatttgcaaccaatgcattgaataaactaacattaaacgaattcactagtggatcccccaactccgcccgttttatgactagaaccaatagtttttaatgccaaatgcactgaaatcccctaatttgcaaagccaaacgccccctatgtgagtaatacggggactttttacccaatttcccacgcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtggccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaagcttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtgattaagtctgaatgtggctctccaatcatttcgattctctaatctcccaatcctctcaaaaggggcagtatcggacacggactgggaggggcgtacacgatagttatatggtacagcagaggcctctgaacacttaggaggagaattcagccggggagagcccctgttgagtaggcttgggagcatattgcaggatgaacatgttagtgatagttctcgcctcttgtcttgcgcgcctaacttttgcgacgcgacacgtcctctttttggaaggcactcaggctgtcctcggggaagatgatcccagaaacgttccggaagggactgtaatcaaatggacaaaagtcctgcggaacgcgtgcaagatgaaggcggccgatgtctgctcttcgcctaactattgctttcatgatttaatttacgacggaggaaagaaagactgcccgcccgcgggacccctgtctgcaaacctggtaattttactaaagcgcggcgaagcttagcttgcctccgattctagcattacatagccggtcagtagatcctgccattcggtagcgcaaccggctacatcttcaaacagtctcacgataaatgcatctctcgttcctgccaatccggaaccgggcataccactcccgcctgccgatttaattctcacaattgggcgatgccggcggggcaaaacgaatgtggatttggcaaaccgacacaggtctgctgtacggactaatatgggcacacccacatcattcttcagatgctccatgcattgttctatgagaaagatccatagggtggaggcagcgtcacgagatcgcccaggcaatcgatcgcattcgtctagtaaagtgacgagagttatcatgcacacacccatgcccacgccttccgaataactggagctgtggaagatcggaaacgtctttttgactgccggtctcgtactactttcgcacaggtgtatacccggacgcgtactatatattttatatcatccaacgtccgaaattacatacgtggcggcgatggaagtagatgttgagtcttcgaaagtaagtgcctcgaatatgggtattgtctgtgaaaatatcgaaagcggtacgacggttgcagaaccgtcgatgtcgccagatactagtaacaatagcttcgataacgaagacttccgtgggcctgaatacgatgtggagataaataccagaaaatctgctaatcttgatcgtatggaatcttcgtgccgtgaacaacgagcggcgtgcgaacttcgaaagtgttcgtgtcctacgtctgccgtgcgcatgcaatacagtattctttcatctctcgctccgggttcagagggtcatgtatatatatgtactagatacggggacgcggaccaaaaaaaatgcatagtgaaggcagtcgttggaggaaagaatcccgggagggaagtggatattttaaaaaccatctcacataaatcaattataaaattaatccatgcctataaatggaaaaatgttgtgtgtatggcaatgcgtgtatatcgttatgatcttttcacatatattgacggagtcggccctatgccccttcaacagatgatctatattcaacgtggactactagaggcgctagcatacatacatgaaaggggcatcattcaccgagacgtaaagacggagaatatattcttggataatcacgaaaatgcagttttgggtgacttcggtgctgcatgccaactaggagattgtatagatacgccccaatgttacggttggagcggaactgtggaaacaaattcgccggaattatctgcacttgatccgtattgcacaaaaacagatatttggagtgccggattggttctatatgagatggcaattaaaaatgtaccattgtttagtaagcaggtgaaaagttcgggatctcagctgagatccataatacggtgcatgcaagtgcatgaactggagtttccccgcaacgattctaccaacctctgtaaacatttcaaacaatatgcggttcgtgtacgaccgccttataccattcctcgagttataagaaatggggggatgccaatggatgttgaatatgtcatttctaaaatgcttacgtttgaccaggagttcagaccttctgctaaggaaatattgaatatgcccctatttactaaggcgccgattaacctgcttaatatcacaccctctgacagtgtctaacggtatacaggcgggagcgggtcgtggcgtcatcatcaccacttgagaatttatattttgaattgttgattgataaattaacctgattcattgagaactgaaacgccatattggtttcttggatatgtctacaacaattagttaaattgctatgttctactgcgagtaacatttgataagttgtaagagacgggcgactcatgtcgaagttgacgaatataaagtacataacgtgtttagaatacccagaatccgaatagtccgcgggggcgtcttctcgcgtgagtaccaaatactgagttgaacttgaaaatgctaaatctgtgacactctttgtgtgatgattattgtcaccacttcgaagatggcttcgacattcatgatgttctggtgtttgtttggaatcgtaatagcgcttgtttcgtccaagtctgacaacaaagaaaatctgaagaattatatcacggataagtcaaccaatattagaatacccacgccattatttgtatcaacggaaaactcttatcccacaaaacatgtaatctacgatgaaaactgtggcttcgctgtactcaatcctataagtgaccccaaatatgtccttttgagccagcttctaatgggaaggcgcaaatatgatgcgacggtcgcgtggtttgttctcggtaaaatgtgtgccagattaatatatttgcgcgaattttataactgctcgacaaatgagccttttggcacatgttctatgagctctcctggatggtgggacaggcgctacgtctcaaccagtttcatttctcgcgacgaattacagctggtttttgcagcgccgtcccgagaattagatggtttatatacgcgcgtagtagttgtcaacggggactttactacggccgatataatgtttaatgttaaagtggcatgtgccttttcaaagactggaatagaagatgatacattatgcaaaccctttcatttctttgccaatgcaacattgcacaatttaaccatgattagatcggtaactcttcgagcgcacgaaagccatttaaaggaatgggtggcacggagaggtggtaacgtccctgcagtgctacttgagtctaccatgtatcatgcatccaatctgcctagaaatttcagggatttctacataaagtctccagatgattataagtataatcacctagatgggccatctgtaatgctcatcactgacagacctagtgaagatttggatgggaggctcgttcaccaaagtgacatttttactactacaagtcctataaaacaggtccggtatgaagagcatcagtcacatacaaagcagtatcctgtaaacaaaatacaagctataatttttttgatagggttaggctcgttcattggaagcatattcgtagttttggtagtatggattatacgcagatattgcaatggagcgcggagtgggggaacgccccccagtcctcgccggtatgtgtataccaggctatgatcacgtgtgaaacttgggcggacctgtatcatatgtacaccgtccctattcgtttatagccagtacgtgttatctgcacatagaggaacatgtgtcatactgggatcgcatgcatggtatgtgtgactctaatattattctgtatcataataaaaacacagtgcatggtatatagaggatcgctggtaagcactacggtagaccaatcggctcagattgcattctttggcatcgataccgttgttaatttatatggcaaagtcttgttcatgggagatcagtatttggaggaaatatactctggaacgatggaaatactcaaatggaatcaagctaaccgctgctattctattgcgcatgcaacatattacgccgactgtcctataatcagttctacggtattcagaggatgccgggacgccgttgtttatactaggccccacagcagaattc

SEQ ID NO: 16 - DNA, 13064 bp, synthetic construct
  misc_feature 1..13064: note = Herpesvirus of turkeys, Infectious laryngotracheitis virus, Gallus gallus, Infectious bursal disease virus, and Feline Herpesvirus
  misc_difference 7401..7403: note = n is a, c, g, or t
  source 1..13064: mol_type = other DNA
gaattccagactaaatgccccggcccaatttgtcaagtgtgcagtcacggaggcgtcgaccgtgtccccggcattaaacaggaaagcgttaaagtttttgaatgttaggtcacaggtacaaacataaatgtttgtacaaacaggtaacaggtacaaacataaatgccccggcataaatgtcccttacggcggatcgaaacgacattaggcatactcgggtaccattttgcattccgatcagcacggatgaaattaggcaggaatgcggtttatattatgcggcattggacaaacgatatggcattgattggcagtttatgaatgtcttcatgttgggcgtaaacggattcctattggttcagaagacaacgacgatatatttagagagaaaaagctacccagcataggataaacacacattgagcattgagagacataggtatcggtatggatgggaaaactacacacgtgaacaccaaacgacttatatactcgagcggtgatactactgagcaagaatgcactgcatctgagccactgaatgaagactgtgatgaaaatgtgaccatcgatggaattggagaagaatatgcgcagttcttcatgtccccgcaatgggtcccaaatctacatcgcttgagcgaggataccaaaaaggtataccgatgtatggtttccaacagactcaattattttccctattatgaggcgttcaggcggtctttgtttgatatgtatatgctaggtcggttggggcgtcgacttaagcgatctgactgggagactattatgcatctgtcaccaacgcaaagtcggcgtctacatagaactttaagatttgtggagcgtagaattatcccatctaacagttatatacgcacatcgggccacgttccgccttcgagggcacttccgacagatacgaatttaaagatggatgaataattaaattggaaagagtaactacattaatcgagcgtcatgacggcgtcccgtgaaaatgggaattttctactcgaaacaccgtgacatttgacagacctggaattgttattctgatatatagtgggtgtgtctggccggcaacatacataatgtgcatgcgaaaccactttttcagtgtacgctgacattgtgcaacacggaggggtagcatctacatacaatatatgttgattaatgattggagaaaaaactatgcagctcgccgatcatatggctaactcgccttcgtctatatggcggaccccgcgggaaaaatcgacgtaccatctgatttacaacaccagtaatgaacatgtcgcatccctgcccagatctgtgcgcccattggcgcggatcgttgtgaatgccgccgaaacacttcaggtcggtatgagagccgggaggccgccatcagcaggagtttggcgagaggtgtttgatagaatgatgacagccttccgtgaccacgagcctactgcgacatttaatgctgcaaatcccattagaaaaatggtcgagacagttctacagaataatgaagagcccccgcggacgcatgctgaaatgggtaatcgccttatgaacattatgtactggtgttgcttgggacacgcaggacaatgctcgatatggcagttgtacgagacgaatcaggccattttaagtttattagatgaagtggttatcggcacaacaaatcccttttgcaccctcgagcaatactggaagccattatgcaccgcaatcgccaacaaggggacctcatcgcttgttgaggatgccaaagtggccgagtacctggttagcatgcgcaaattgatataacataggcacgctctgatgttacagaccacaataccgcatacatttattgtaaggttgttaataaaggtttattctatgtaagactacaatactttcgacattgcttgtatacatattaaatactttctcaagttcctattacataaaatgggatctatcattacattcgttaagagtctggataattttactgtttgccagcttcgatcttggaacgtactgtggatagtgccttacttggaatcgtgaaaatttgaaacgtccattatttggatatcttccggttgtcccatatcccgccctggtaccgctcggataccttgcccgtatggattcgtattgacagtcgcgcaatcggggaccaacaacgcgtgggtccacactcattcggaaattttccgatgattctgaatatttattgccgctcgttacgagtcgttggacatatctgtaatacatttcttcttctgaaggatcgctgcacatttgatctatacattggccaggatgttcaagtctcagatgttgcattctggcacagcacaactttatggcatttccgatgtaatcgtccggcagccctgggggagttctatattcgcatattgggatggtaaggacaatagcagatctcgcaacctccagggaggctataataacgtttttaaaggatggatttctcataaaaatctgtcgcaaattacactgagaatatcctttactagcgccgattgagagcatcgtcgtccaattttctaaatggaaagaaaacaaggcgggcaagagtgttccaaacattttcattttcggcgaatctctcaaatcccatggcgtgcaattgattgcaaaattggcacttccgttcacgtttgtatctccaaactctaagacacttttaattgaaaaactacgttctagtgtggaaagaaacctataggcagaccatagaactatttgacaccacatatctttttgtatgtcaaactgaccatgatcgtatgttgctgaatgcactagggcaattcgctcgcgcgactccatacattgaataattccacacgtcagctcatcggttagcaaggtccagtagttgaagtcatttatttttccccgcggctggccaaatctacctctgggaatatccaagttgtcgaatatgatcgcaccggctctggtcatggtgaaggaactgtagcataaagacgcaggtatcataggggtaatatttttttattcactcacatactaaaagtaacgcatattagcaccatgtatgggctatcaattgacatttgcgtagcactacatcacgattatgtacaacataatgggacaacatatggcaagtagatgcaatttcctcacactagttgggtttatctactattgaattttcccctatctgtgatacacttgggagcctctacaagcatattgccatcatgtacgtttttatctactgtcttaacgcccatgggaacggaggcgtcgtcgtcatgtattggacggcaacataggcagcaacacaaattgcgtttaggtggggtgcatgtggactcgataccaagcccctgcagctggggaacgtctggtggagagccgataatttgatatacgcacgccatattactgtcgttgaagtacgccttatcttctatgttttcaaatttaggttcccaagtggacgtgagaagtgtttgtatctcacatggaatggcccaaggcattccagcccaggtgcctggtactttaatggcaaacaaacgttttggtagaggtattgattctattgcagttctgcagatatctgcagccccgagtatccacaggctatacgatacgttatcggaggcaagcttaattaagtaccgagctcgaattggcgcgcccgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtgattaagtctgaatgtggctctccaatcatttcgattctctaatctcccaatcctctcaaaaggggcagtatcggacacggactgggaggggcgtacacgatagttatatggtacagcagaggcctctgaacacttaggaggagaattcagccggggagagcccctgttgagtaggcttgggagcatattgcaggatgaacatgttagtgatagttctcgcctcttgtcttgcgcgcctaacttttgcgacgcgacacgtcctctttttggaaggcactcaggctgtcctcggggaagatgatcccagaaacgttccggaagggactgtaatcaaatggacaaaagtcctgcggaacgcgtgcaagatgaaggcggccgatgtctgctcttcgcctaactattgctttcatgatttaatttacgacggaggaaagaaagactgcccgcccgcgggacccctgtctgcaaacctggtaattttactaaagcgcggcgggcgcgccggatcagatctccatggtcgaggtgagccccacgttctgcttcactctccccatctcccccccctccccacccccaattttgtatttatttattttttaattattttgtgcagcgatgggggcgggggggggggnnncgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcagatctggatctatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaagttgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtggccgcagataatgggctgacggccggcaccgacaatcttatgccattcaatcttgtcattccaaccaatgagataacccagccgatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaagatccgatctctcgattaattaacaataaacatagcatacgttatgacatggtctaccgcgtcttatatggggacgacaagcttgcctccgattctagcattacatagccggtcagtagatcctgccattcggtagcgcaaccggctacatcttcaaacagtctcacgataaatgcatctctcgttcctgccaatccggaaccgggcataccactcccgcctgccgatttaattctcacaattgggcgatgccggcggggcaaaacgaatgtggatttggcaaaccgacacaggtctgctgtacggactaatatgggcacacccacatcattcttcagatgctccatgcattgttctatgagaaagatccatagggtggaggcagcgtcacgagatcgcccaggcaatcgatcgcattcgtctagtaaagtgacgagagttatcatgcacacacccatgcccacgccttccgaataactggagctgtggaagatcggaaacgtctttttgactgccggtctcgtactactttcgcacaggtgtatacccggacgcgtactatatattttatatcatccaacgtccgaaattacatacgtggcggcgatggaagtagatgttgagtcttcgaaagtaagtgcctcgaatatgggtattgtctgtgaaaatatcgaaagcggtacgacggttgcagaaccgtcgatgtcgccagatactagtaacaatagcttcgataacgaagacttccgtgggcctgaatacgatgtggagataaataccagaaaatctgctaatcttgatcgtatggaatcttcgtgccgtgaacaacgagcggcgtgcgaacttcgaaagtgttcgtgtcctacgtctgccgtgcgcatgcaatacagtattctttcatctctcgctccgggttcagagggtcatgtatatatatgtactagatacggggacgcggaccaaaaaaaatgcatagtgaaggcagtcgttggaggaaagaatcccgggagggaagtggatattttaaaaaccatctcacataaatcaattataaaattaatccatgcctataaatggaaaaatgttgtgtgtatggcaatgcgtgtatatcgttatgatcttttcacatatattgacggagtcggccctatgccccttcaacagatgatctatattcaacgtggactactagaggcgctagcatacatacatgaaaggggcatcattcaccgagacgtaaagacggagaatatattcttggataatcacgaaaatgcagttttgggtgacttcggtgctgcatgccaactaggagattgtatagatacgccccaatgttacggttggagcggaactgtggaaacaaattcgccggaattatctgcacttgatccgtattgcacaaaaacagatatttggagtgccggattggttctatatgagatggcaattaaaaatgtaccattgtttagtaagcaggtgaaaagttcgggatctcagctgagatccataatacggtgcatgcaagtgcatgaactggagtttccccgcaacgattctaccaacctctgtaaacatttcaaacaatatgcggttcgtgtacgaccgccttataccattcctcgagttataagaaatggggggatgccaatggatgttgaatatgtcatttctaaaatgcttacgtttgaccaggagttcagaccttctgctaaggaaatattgaatatgcccctatttactaaggcgccgattaacctgcttaatatcacaccctctgacagtgtctaacggtatacaggcgggagcgggtcgtggcgtcatcatcaccacttgagaatttatattttgaattgttgattgataaattaacctgattcattgagaactgaaacgccatattggtttcttggatatgtctacaacaattagttaaattgctatgttctactgcgagtaacatttgataagttgtaagagacgggcgactcatgtcgaagttgacgaatataaagtacataacgtgtttagaatacccagaatccgaatagtccgcgggggcgtcttctcgcgtgagtaccaaatactgagttgaacttgaaaatgctaaatctgtgacactctttgtgtgatgattattgtcaccacttcgaagatggcttcgacattcatgatgttctggtgtttgtttggaatcgtaatagcgcttgtttcgtccaagtctgacaacaaagaaaatctgaagaattatatcacggataagtcaaccaatattagaatacccacgccattatttgtatcaacggaaaactcttatcccacaaaacatgtaatctacgatgaaaactgtggcttcgctgtactcaatcctataagtgaccccaaatatgtccttttgagccagcttctaatgggaaggcgcaaatatgatgcgacggtcgcgtggtttgttctcggtaaaatgtgtgccagattaatatatttgcgcgaattttataactgctcgacaaatgagccttttggcacatgttctatgagctctcctggatggtgggacaggcgctacgtctcaaccagtttcatttctcgcgacgaattacagctggtttttgcagcgccgtcccgagaattagatggtttatatacgcgcgtagtagttgtcaacggggactttactacggccgatataatgtttaatgttaaagtggcatgtgccttttcaaagactggaatagaagatgatacattatgcaaaccctttcatttctttgccaatgcaacattgcacaatttaaccatgattagatcggtaactcttcgagcgcacgaaagccatttaaaggaatgggtggcacggagaggtggtaacgtccctgcagtgctacttgagtctaccatgtatcatgcatccaatctgcctagaaatttcagggatttctacataaagtctccagatgattataagtataatcacctagatgggccatctgtaatgctcatcactgacagacctagtgaagatttggatgggaggctcgttcaccaaagtgacatttttactactacaagtcctataaaacaggtccggtatgaagagcatcagtcacatacaaagcagtatcctgtaaacaaaatacaagctataatttttttgatagggttaggctcgttcattggaagcatattcgtagttttggtagtatggattatacgcagatattgcaatggagcgcggagtgggggaacgccccccagtcctcgccggtatgtgtataccaggctatgatcacgtgtgaaacttgggcggacctgtatcatatgtacaccgtccctattcgtttatagccagtacgtgttatctgcacatagaggaacatgtgtcatactgggatcgcatgcatggtatgtgtgactctaatattattctgtatcataataaaaacacagtgcatggtatatagaggatcgctggtaagcactacggtagaccaatcggctcagattgcattctttggcatcgataccgttgttaatttatatggcaaagtcttgttcatgggagatcagtatttggaggaaatatactctggaacgatggaaatactcaaatggaatcaagctaaccgctgctattctattgcgcatgcaacatattacgccgactgtcctataatcagttctacggtattcagaggatgccgggacgccgttgtttatactaggccccacagcagaattc

SEQ ID NO: 17 - DNA, 13017 bp, synthetic construct
  misc_feature 1..13017: note = Herpesvirus of turkeys, Infectious laryngotracheitis virus, human cytomegalovirus, Infectious bursal disease virus, and Herpes simplex virus
  source 1..13017: mol_type = other DNA
gaattccagactaaatgccccggcccaatttgtcaagtgtgcagtcacggaggcgtcgaccgtgtccccggcattaaacaggaaagcgttaaagtttttgaatgttaggtcacaggtacaaacataaatgtttgtacaaacaggtaacaggtacaaacataaatgccccggcataaatgtcccttacggcggatcgaaacgacattaggcatactcgggtaccattttgcattccgatcagcacggatgaaattaggcaggaatgcggtttatattatgcggcattggacaaacgatatggcattgattggcagtttatgaatgtcttcatgttgggcgtaaacggattcctattggttcagaagacaacgacgatatatttagagagaaaaagctacccagcataggataaacacacattgagcattgagagacataggtatcggtatggatgggaaaactacacacgtgaacaccaaacgacttatatactcgagcggtgatactactgagcaagaatgcactgcatctgagccactgaatgaagactgtgatgaaaatgtgaccatcgatggaattggagaagaatatgcgcagttcttcatgtccccgcaatgggtcccaaatctacatcgcttgagcgaggataccaaaaaggtataccgatgtatggtttccaacagactcaattattttccctattatgaggcgttcaggcggtctttgtttgatatgtatatgctaggtcggttggggcgtcgacttaagcgatctgactgggagactattatgcatctgtcaccaacgcaaagtcggcgtctacatagaactttaagatttgtggagcgtagaattatcccatctaacagttatatacgcacatcgggccacgttccgccttcgagggcacttccgacagatacgaatttaaagatggatgaataattaaattggaaagagtaactacattaatcgagcgtcatgacggcgtcccgtgaaaatgggaattttctactcgaaacaccgtgacatttgacagacctggaattgttattctgatatatagtgggtgtgtctggccggcaacatacataatgtgcatgcgaaaccactttttcagtgtacgctgacattgtgcaacacggaggggtagcatctacatacaatatatgttgattaatgattggagaaaaaactatgcagctcgccgatcatatggctaactcgccttcgtctatatggcggaccccgcgggaaaaatcgacgtaccatctgatttacaacaccagtaatgaacatgtcgcatccctgcccagatctgtgcgcccattggcgcggatcgttgtgaatgccgccgaaacacttcaggtcggtatgagagccgggaggccgccatcagcaggagtttggcgagaggtgtttgatagaatgatgacagccttccgtgaccacgagcctactgcgacatttaatgctgcaaatcccattagaaaaatggtcgagacagttctacagaataatgaagagcccccgcggacgcatgctgaaatgggtaatcgccttatgaacattatgtactggtgttgcttgggacacgcaggacaatgctcgatatggcagttgtacgagacgaatcaggccattttaagtttattagatgaagtggttatcggcacaacaaatcccttttgcaccctcgagcaatactggaagccattatgcaccgcaatcgccaacaaggggacctcatcgcttgttgaggatgccaaagtggccgagtacctggttagcatgcgcaaattgatataacataggcacgctctgatgttacagaccacaataccgcatacatttattgtaaggttgttaataaaggtttattctatgtaagactacaatactttcgacattgcttgtatacatattaaatactttctcaagttcctattacataaaatgggatctatcattacattcgttaagagtctggataattttactgtttgccagcttcgatcttggaacgtactgtggatagtgccttacttggaatcgtgaaaatttgaaacgtccattatttggatatcttccggttgtcccatatcccgccctggtaccgctcggataccttgcccgtatggattcgtattgacagtcgcgcaatcggggaccaacaacgcgtgggtccacactcattcggaaattttccgatgattctgaatatttattgccgctcgttacgagtcgttggacatatctgtaatacatttcttcttctgaaggatcgctgcacatttgatctatacattggccaggatgttcaagtctcagatgttgcattctggcacagcacaactttatggcatttccgatgtaatcgtccggcagccctgggggagttctatattcgcatattgggatggtaaggacaatagcagatctcgcaacctccagggaggctataataacgtttttaaaggatggatttctcataaaaatctgtcgcaaattacactgagaatatcctttactagcgccgattgagagcatcgtcgtccaattttctaaatggaaagaaaacaaggcgggcaagagtgttccaaacattttcattttcggcgaatctctcaaatcccatggcgtgcaattgattgcaaaattggcacttccgttcacgtttgtatctccaaactctaagacacttttaattgaaaaactacgttctagtgtggaaagaaacctataggcagaccatagaactatttgacaccacatatctttttgtatgtcaaactgaccatgatcgtatgttgctgaatgcactagggcaattcgctcgcgcgactccatacattgaataattccacacgtcagctcatcggttagcaaggtccagtagttgaagtcatttatttttccccgcggctggccaaatctacctctgggaatatccaagttgtcgaatatgatcgcaccggctctggtcatggtgaaggaactgtagcataaagacgcaggtatcataggggtaatatttttttattcactcacatactaaaagtaacgcatattagcaccatgtatgggctatcaattgacatttgcgtagcactacatcacgattatgtacaacataatgggacaacatatggcaagtagatgcaatttcctcacactagttgggtttatctactattgaattttcccctatctgtgatacacttgggagcctctacaagcatattgccatcatgtacgtttttatctactgtcttaacgcccatgggaacggaggcgtcgtcgtcatgtattggacggcaacataggcagcaacacaaattgcgtttaggtggggtgcatgtggactcgataccaagcccctgcagctggggaacgtctggtggagagccgataatttgatatacgcacgccatattactgtcgttgaagtacgccttatcttctatgttttcaaatttaggttcccaagtggacgtgagaagtgtttgtatctcacatggaatggcccaaggcattccagcccaggtgcctggtactttaatggcaaacaaacgttttggtagaggtattgattctattgcagttctgcagatatctgcagccccgagtatccacaggctatacgatacgttatcggaggcaagcttgttaattaagtcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtgattaagtctgaatgtggctctccaatcatttcgattctctaatctcccaatcctctcaaaaggggcagtatcggacacggactgggaggggcgtacacgatagttatatggtacagcagaggcctctgaacacttaggaggagaattcagccggggagagcccctgttgagtaggcttgggagcatattgcaggatgaacatgttagtgatagttctcgcctcttgtcttgcgcgcctaacttttgcgacgcgacacgtcctctttttggaaggcactcaggctgtcctcggggaagatgatcccagaaacgttccggaagggactgtaatcaaatggacaaaagtcctgcggaacgcgtgcaagatgaaggcggccgatgtctgctcttcgcctaactattgctttcatgatttaatttacgacggaggaaagaaagactgcccgcccgcgggacccctgtctgcaaacctggtaattttactaaagcgcggcgaaagcttaggtcaattccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggcgcgccggatctatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaagttgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtggccgcagataatgggctgacggccggcaccgacaatcttatgccattcaatcttgtcattccaaccaatgagataacccagccgatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaagatccataattgattgacgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgaagcttgcctccgattctagcattacatagccggtcagtagatcctgccattcggtagcgcaaccggctacatcttcaaacagtctcacaataaatgcatctctcgttcctgccaatccggaaccgggcataccactcccgcctgccgatttaattctcacaattgggcgatgccggcggggcaaaacgaatgtggatttggcaaaccgacacaggtctgctgtacggactaatatgggcacacccacatcattcttcagatgctccatgcattgttctatgagaaagatccatagggtggaggcagcgtcacgagatcgcccaggcaatcgatcgcattcgtctagtaaagtgacgagagttatcatgcacacacccatgcccacgccttccgaataactggagctgtggaagatcggaaacgtctttttgactgccggtctcgtactactttcgcacaggtgtatacccggacgcgtactatatattttatatcatccaacgtccgaaattacatacgtggcggcgatggaagtagatgttgagtcttcgaaagtaagtgcctcgaatatgggtattgtctgtgaaaatatcgaaagcggtacgacggttgcagaaccgtcgatgtcgccagatactagtaacaatagcttcgataacgaagacttccgtgggcctgaatacgatgtggagataaataccagaaaatctgctaatcttgatcgtatggaatcttcgtgccgtgaacaacgagcggcgtgcgaacttcgaaagtgttcgtgtcctacgtctgccgtgcgcatgcaatacagtattctttcatctctcgctccgggttcagagggtcatgtatatatatgtactagatacggggacgcggaccaaaaaaaatgcatagtgaaggcagtcgttggaggaaagaatcccgggagggaagtggatattttaaaaaccatctcacataaatcaattataaaattaatccatgcctataaatggaaaaatgttgtgtgtatggcaatgcgtgtatatcgttatgatcttttcacatatattgacggagtcggccctatgccccttcaacagatgatctatattcaacgtggactactagaggcgctagcatacatacatgaaaggggcatcattcaccgagacgtaaagacggagaatatattcttggataatcacgaaaatgcagttttgggtgacttcggtgctgcatgccaactaggagattgtatagatacgccccaatgttacggttggagcggaactgtggaaacaaattcgccggaattatctgcacttgatccgtattgcacaaaaacagatatttggagtgccggattggttctatatgagatggcaattaaaaatgtaccattgtttagtaagcaggtgaaaagttcgggatctcagctgagatccataatacggtgcatgcaagtgcatgaactggagtttccccgcaacgattctaccaacctctgtaaacatttcaaacaatatgcggttcgtgtacgaccgccttataccattcctcgagttataagaaatggggggatgccaatggatgttgaatatgtcatttctaaaatgcttacgtttgaccaggagttcagaccttctgctaaggaaatattgaatatgcccctatttactaaggcgccgattaacctgcttaatatcacaccctctgacagtgtctaacggtatacaggcgggagcgggtcgtggcgtcatcatcaccacttgagaatttatattttgaattgttgattgataaattaacctgattcattgagaactgaaacgccatattggtttcttggatatgtctacaacaattagttaaattgctatgttctactgcgagtaacatttgataagttgtaagagacgggcgactcatgtcgaagttgacgaatataaagtacataacgtgtttagaatacccagaatccgaatagtccgcgggggcgtcttctcgcgtgagtaccaaatactgagttgaacttgaaaatgctaaatctgtgacactctttgtgtgatgattattgtcaccacttcgaagatggcttcgacattcatgatgttctggtgtttgtttggaatcgtaatagcgcttgtttcgtccaagtctgacaacaaagaaaatctgaagaattatatcacggataagtcaaccaatattagaatacccacgccattatttgtatcaacggaaaactcttatcccacaaaacatgtaatctacgatgaaaactgtggcttcgctgtactcaatcctataagtgaccccaaatatgtccttttgagccagcttctaatgggaaggcgcaaatatgatgcgacggtcgcgtggtttgttctcggtaaaatgtgtgccagattaatatatttgcgcgaattttataactgctcgacaaatgagccttttggcacatgttctatgagctctcctggatggtgggacaggcgctacgtctcaaccagtttcatttctcgcgacgaattacagctggtttttgcagcgccgtcccgagaattagatggtttatatacgcgcgtagtagttgtcaacggggactttactacggccgatataatgtttaatgttaaagtggcatgtgccttttcaaagactggaatagaagatgatacattatgcaaaccctttcatttctttgccaatgcaacattgcacaatttaaccatgattagatcggtaactcttcgagcgcacgaaagccatttaaaggaatgggtggcacggagaggtggtaacgtccctgcagtgctacttgagtctaccatgtatcatgcatccaatctgcctagaaatttcagggatttctacataaagtctccagatgattataagtataatcacctagatgggccatctgtaatgctcatcactgacagacctagtgaagatttggatgggaggctcgttcaccaaagtgacatttttactactacaagtcctataaaacaggtccggtatgaagagcatcagtcacatacaaagcagtatcctgtaaacaaaatacaagctataatttttttgatagggttaggctcgttcattggaagcatattcgtagttttggtagtatggattatacgcagatattgcaatggagcgcggagtgggggaacgccccccagtcctcgccggtatgtgtataccaggctatgatcacgtgtgaaacttgggcggacctgtatcatatgtacaccgtccctattcgtttatagccagtacgtgttatctgcacatagaggaacatgtgtcatactgggatcgcatgcatggtatgtgtgactctaatattattctgtatcataataaaaacacagtgcatggtatatagaggatcgctggtaagcactacggtagaccaatcggctcagattgcattctttggcatcgataccgttgttaatttatatggcaaagtcttgttcatgggagatcagtatttggaggaaatatactctggaacgatggaaatactcaaatggaatcaagctaaccgctgctattctattgcgcatgcaacatattacgccgactgtcctataatcagttctacggtattcagaggatgccgggacgccgttgtttatactaggccccacagcagaattc

SEQ ID NO: 18 - DNA, 15252 bp, synthetic construct
  misc_feature 1..15252: note = Herpesvirus of turkeys, murine cytomegalovirus, Infectious bursal disease virus, Simian vacuolating virus 40, and Infectious laryngotracheitis virus
  source 1..15252: mol_type = other DNA
ggcgcgccactggagaacggcatgaccgcaaaaggcgttgtagagatcgatcccacgaactctcaggcgatcgtgtcagtcgccataaacagcgacgatcgtctccaggatctgaacggttttcttctcaacgatcatcagtatatgaggaactgaacctgatatttagccgagggaaacgcaggttaaaaaccctatcaagcgattgcgattttcgcgtatctagtaaaaatagatgggcttcggtactagccttcgccgccaactctgaatatgcccttcgtggacctcatataacatggcattgtttgttggatgcggggccggaattaagaagaacattcgaaatacgagcaaaaatttcggccctggcatgtgctgcgcgagaatcggtacttcggggagaaagttttatcggagctttgggtagtgcagaggaaactctatcttggttgaaaatgcatgcgaccctgcacttgattctggttaaccacgatccaatttttaagacggctggcgcggtcctagataacctccgcttaaaactagccccaatattgatgtgcagatataacacagaaaaacgatcaatggaagacatgctacggcggtcatctcccgaagacatcaccgattccctaacaatgtgcctgattatgttatcgcgcattcgtcgtaccatgcgcaccgcaggaaataaatatagctatatgatagatccaatgaatcgtatgtctaattacactccaggcgaatgtatgacaggtatattgcgatatattgacgaacatgctagaaggtgtcctgatcacatatgtaatttgtatatcacatgtacacttatgccgatgtatgtgcacgggcgatatttctattgtaattcatttttttgttagtaaactaccacaggctgtccggaaatctaagttaatgaataaagtagatggttaatactcattgcttagaattggactacttttaattctctttaatgttcgtattaaataaaaacatctttaataaacttcagcctcttcgcttattgtagaaattgagtattcaaaatcatgttcaaagccgtcttcggagagtgtactcgccacggtggttggaacatcactatgtctacacgtcaaatttaagcacgtcaggtctgtcgaggacaagaaatggttaactagtgtttcaattattcttataaacgttaagcattgtaagccccccggccgtccgcagcaacaatttactagtatgccgtgggctccgggactatcacggatgtccaattcgcacatgcatataatttttctagggtctctcatttcgagaaatcttcggggatccatcagcaatgcgggctgtagtcccgattcccgtttcaaatgaaggtgctccaacacggtcttcaaagcaaccggcataccagcaaacacagactgcaactccccgctgcaatgattggttataaacagtaatctgtcttctggaagtatatttcgcccgacaatccacggcgcccccaaagttaaaaaccatccatgtgtatttgcgtcttctctgttaaaagaatattgactggcattttcccgttgaccgccagatatccaaagtacagcacgatgttgcacggacgactttgcagtcaccagccttcctttccacccccccaccaacaaaatgtttatcgtaggacccatatccgtaataaggatgggtctggcagcaaccccataggcgcctcggcgtggtagttctcgaggccttaagcttaaggatcccccaactccgcccgttttatgactagaaccaatagtttttaatgccaaatgcactgaaatcccctaatttgcaaagccaaacgccccctatgtgagtaatacggggactttttacccaatttcccacgcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtggccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtgattaagtctgaatgtggctctccaatcatttcgattctctaatctcccaatcctctcaaaaggggcagtatcggacacggactgggaggggcgtacacgatagttatatggtacagcagaggcctctgaacacttaggaggagaattcagccggggagagcccctgttgagtaggcttgggagcatattgcaggatgaacatgttagtgatagttctcgcctcttgtcttgcgcgcctaacttttgcgacgcgacacgtcctctttttggaaggcactcaggctgtcctcggggaagatgatcccagaaacgttccggaagggactgtaatcaaatggacaaaagtcctgcggaacgcgtgcaagatgaaggcggccgatgtctgctcttcgcctaactattgctttcatgatttaatttacgacggaggaaagaaagactgcccgcccgcgggacccctgtctgcaaacctggtaattttactaaagcgcggcgaaagcttcccgggttaattaaggccctcgaggatacatccaaagaggttgagtattctctctacacttcttgttaaatggaaagtgcatttgcttgttcttacaatcggcccgagtctcgttcacagcgcctcgttcacacttaaaccacaaatagtctacaggctatatgggagccagactgaaactcacatatgactaatattcgggggtgttagtcacgtgtagcccattgtgtgcatataacgatgttggacgcgtccttattcgcggtgtacttgatactatggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccggtggagtatacgggaaactaaatgttcatagaggtctttgggctatatgttattaaataaaataattgaccagtgaacaatttgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgatgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccattatcttcagtggagcgtagacgggcgaggctaattatgtgacatagcaacactgcatgtatgtttttataaatcaataagagtacataatttattacgtatcatttccgtttgtaatatactgtatacatcatccacactattagtcagcactagcgcgcgggcgcacgttacaatagcagcgtgcccgttatctatattgtccgatatttacacataacatttcatcgacatgattaaatacctaagtactgcacacagatgtttaatgtatatcgtcatataaattatatcgctaggacagacccaaacgacctttatcccaaacagtcagatcctcttctcaagtgtcgatttctgttatggaatatgcataccctggcccagaaattgcacgcacgagcgtagtgaatgcgtcattggttttacatttaaaggctaaatgcacaaattctttagacgacagcacatcgttaaatagcatctctagcgttcttatgaatgctaagcattggagtcctcctggtcggccacaataacagctgagtatcataccctgagctccggggttgtcgcacatagcggattcgtataaacataggattttccgcgaatccatcagttgcaaaaatctgttaggctccatcaacaacgctggatttacttcagatccacgcgtaaagtaatggtgctcgaataccgtttttagagttgtcggcatttcaaggaacaaagaattcatttcttcattgcaacgacgcgccagaaatcccaagacctctttgggtagtatgttcttgcctataaaacacggcgttccaagtgccaggaaccacgcatgtgttactgttggggcgtattcagaaataaagcggggtttatgcggcttttgaagctcggatatccaaagtatcgcttgctgatgaacgagcgatgtagctgttacaaaacctcctttccatcctccagtcaacataatatttatcggcctacctatgtccgtaataagtattggtcgggcaattattccgtatgaggtcttgcaggaataagctcttagggacagccagcttggatatggtgcgaaacagaccttctcggcttcagaatgtcgctccgcagtctcttcgtgtcggtgcatcttagatccaccatcaatgtgtgcagcattgactcccgcccgtcgaatattccttttgttacgatgcagtaatgagcacgatcatgggcggggcgatgacgttctatttgcatgtctgcgaacaatttgcgtcagtcatacagctatggagtgggccatttctggccgtcaacttaaaaacgcgaaccgcagacatatgtatttgcatgcaaagacgtatcttcgtatttctgggcatcttcaaatgctctggccaatatggcaatgaatttggattcgtttgacgccgatggtatgcagtgcaaatgtgccaatagcccacatccgaaaaagttatttgtcatacaagcaggtgttaagtagcaatcacataaaggcaccagacgcctcatggcatcataatgaatagctccttctccccactggaaccactgacaaaatctgcgagtatattccgcaaaccacattttatttctcatagaaactaccctaaatccttttaacgggaagaagaatcctagatagtgcttgaagtcatgactgttactgctgcaataacactgtatattatttataaattccgtttgtctaggtatctgatgtaggcattccgatccctttactattgcgtcttcacgaccaaatgggaatgcgccaaaatccccacacctcatcaccctggaggcagattgtgtattattaatatccgccgattgaagcacaaaacggtacggtactgttcctaattctggtatagattctatggtcaaaagtctgcatatccccgacattgccatgagatcacacagtccaagtagcatgtttattgagtcactcagactgtcaacgtccctcgccgcaccaccaatcgaaaataaagtatctacgcaagttatagctccgcattttctatcgctagcagcaatcgcgacgcaaaacataaaggccatgttgggatttgaactctctggggggcttgttatcttctgcaccgtcgcagtcgcagttttccgaaatttatgtctaatatattttccggccgtgctccaatcggccgaaaagaatctgcgtattaccagactcattgacgggccgataaagaccataaaacaaaattcctgtgcactccctcctccagttttgccatcgtccaagtcccgtaactttttttgcgtttcgaggagcaagcgttcgttatccctacccacacttgttttccaccgttttcttattataagcggttgtatcgccaacgcgtcaccgcaggttgtcacatacagtgatggcatacttgaacgtgcaacaacgcgctcgctttgcaaatctaagtcattgaccatcaaatcgcgttgagaggatagccaggcatcttttttcctagtatggtgacggtgcagccaccccaactcagttcttgtaaaaaaagctattggcgggaatttatgttctgaggtgcattctatatttatgagtccatcaaatgccattaaccagattcgtattttttcgctcgacccggcatcactatggatacaatacctttctatggcccatttcagctctcgaaccaaccacacggacaattgactaacataagtatgatctttatcacagtcgcacccatctgagttatatttatggcatccgagcgctcttactgtacggtcggatacacccatggtttttcctttatatagtcgggttatagtctgtcgggtttggcggtagcacggagtagtttgatttttaagaatcgaaaaccggcttggagagaccactgtcgaatatttgtccgtatactctacacgtgagtgttgtccattcctaggtatattcatctgttcggataccttcaattgctgttcaggcataaccttaaagcatatgttatgttgtacatcaaaacttggtgagttatgttcgattgccgcgcataaagaatcgtacatgagcgtttctgctaacatactatctatattctcacacgcccctgcatatactgttcctattccaaattcacgttttgccccatcggctatctgctcccaaaaagttgtaatataggtgccgctgggtgcgaaattttcatcagttgtattcctgataaactgaatcactttacataatttttgccacatatctgcgtgcagccatagtatcgaacccgtgggctcggagacgacagtgcgtacaatgggtattttacctttccccaacaaaataatggtatacaagttaggtccgtacctagaccttaatgtttccaattcttctgaatcactgcactctcgtaggggagtaacggtaataatttcgtctctgagccccgttttgcgttgaaaactaatcacattagataatgtgcaatcggtttcttttatccggatacatctaagtattatgacatcggtggtcattgtttccatcaacgaccatcttttacgatcgcccatactactcatggacgttgtcggtgttgaaaaatcaccagaattgcaacggatctctgggtaccatgctgctgatggaattggcggttttaattgttgtttcagtctattattgctatctttggcggggttgaataatgtggggggagagtgattgcaggaatccgaatgggtcaataaaacgaccgtgctccgttctgccggcgccgatccgattgaagctatatacttcgcttctctccccacttttccaatttgatccggaaataaaacggccccggacaacagtatcgtacgatccggatccggatcctgcttgcctacagaagaatcaacatctcgccccaatattctggtcaaaactggctcgctcatggcaacgcggacgtttcccccggtggccagtcttaatggttaatgttcttttcggcaatcttatacatcagcgggttgcgtgaatactggtcacagttcagtcatttactacacaccagcaatacgacgacggacagtaccgtcccgacgaacgcgacgcccaaaattgctatcgcgaccgcgtccgaggcgatgtcgtacgggcggtgcggggttggatcctcggcaaagagatcctcgtaattcggcggtgggagcggagggtaaagacgcgggtggggatctccctccggaccgcgcgccgggcgcggttcgaaaatgctttccgcctcgctcagtgtcaacgccaagtattcgggcgggctgggggccggaatatctcccgcgacttcttctatcggcgcggaattggagtcgcggtcgtggcgcgcttctagcgtcgtcaacggaagtccattttcggggtctcccggtgggcgttcagcgtccatcgtcgtatatgctctaacacacgtctcgctatattaaaaaaaagaagagtatcggtcagtgtcgagtgtcgccgacaatgtcgcgagttctcggcgatttaatttttggaactgctccctatgaatcccgtaactgtagcgcccgcgcagaaagccgccatcagaccaactacgtgtctgttcgatgtttgcccgccgatcgctttaccgattaaggttccggcgagaaatgacatgctcgatccaagaacaaagtttttcgcggtaaacaacaacatagttaccgtgcgagatggagaaaccacatctcccgaattagtagaggaaagcccgcgctgtcggtttggggacatatcgatcttttttgtgtttttcctaggacccttttgccagatcgtacaaagtcgcgtcttatgagcggacgttcttactgcagctcggtaggagtggggcagggttagatttcgtcggcgtttcggcccccgtatgcgccgcgccaccctcttcgccgagctctttatgcgcggtgggggtgagcgcttccggagttgcgatctccgatctcgagccgcagcccggcggtgtctctttcagtggagcgttagcgccatcatgtggttcgtggcggtggaaaggctattatgtgttaggggagagaccacgtgatcggcatgcaaatgagcaaggcgaacgcgtcagcgttcgcactgcgaaccaataatatatatattatactattggctttaggtgcgaacgtccggctagtccaatagcggggtcgcgtttcgtaccacgtgttatagaccgccctaaactcgcactcgggggtccggccgcgcccagacagggcggagacgtgccacaggggctttaaaacaccgcttcgggcaccgttcatctcggcgcgcc

SEQ ID NO: 19 - DNA, 199 bp, Simian virus 40
  source 1..199: mol_type = genomic DNA
agcttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcg